(19) United States
(12) Patent Application Publication
(10) Pub. No.: US 2024/0226270 A1
(43) Pub. Date: Jul. 11, 2024

(54) Novel Vaccine Compositions for Porcine Epidemic Diarrhea Virus and Porcine Deltacoronavirus

(71) Applicant: Zoetis Services LLC, Parsippany, NJ (US)

(72) Inventors: Jacqueline Gayle Marx, Portage, MI (US); John Morgan Hardham, Kalamazoo, MI (US); Paul J. Dominowski, Kalamazoo, MI (US); Vicki Jon Rapp Gabrielson, Kalamazoo, MI (US); Monica Balasch Sanuy, Barcelona (ES); Marta Cabana Sumsi, Barcelona (ES); Laia PIaja Dilmé, Girona (ES); Alicia URNIZA Hostench, Girona (ES); Oscar Romero Galindo, White Plains, NY (US)

(21) Appl. No.: 17/177,724

(22) Filed: Feb. 17, 2021

Related U.S. Application Data
(63) Continuation of application No. 16/282,953, filed on Feb. 22, 2019, now Pat. No. 10,953,088.
Application No. 16/282,953 is a continuation of application No. 15/324,908, filed on Jan. 9, 2017, now Pat. No. 10,251,950.
(60) Provisional application No. 62/143,412, filed on Apr. 6, 2015.
(60) Provisional application No. 62/121,193, filed on Feb. 26, 2015.
(60) Provisional application No. 62/115,806, filed on Feb. 13, 2015.
(60) Provisional application No. 62/102,712, filed on Jan. 13, 2015.
(60) Provisional application No. 62/093,657, filed on Dec. 18, 2014.
(60) Provisional application No. 62/046,256, filed on Sep. 5, 2014.
(60) Provisional application No. 62/037,403, filed on Aug. 14, 2014.
(60) Provisional application No. 62/023,302, filed on Jul. 11, 2014.

(51) Int. Cl. A61K 39/215; A61K 39/00; A61K 39/12; C12N 7/00

(52) U.S. Cl. CPC A61K 39/215; A61K 39/12; C12N 7/00; A61K 2039/5252; A61K 2039/552; A61K 2039/55505; A61K 2039/55566; A61K 2039/575; A61K 2039/70; C12N 2770/20022; C12N 2770/20034; C12N 2770/20063; C12N 2770/20071

(57) ABSTRACT

The present invention is directed to novel immunogenic compositions that protect swine from disease caused by porcine epidemic diarrhea virus (PEDV). The present invention is also directed to novel immunogenic compositions that protect swine from disease caused by porcine deltacoronavirus (PDCoV), alone or as combination vaccine to protect against PEDV. The compositions of the invention provide killed viruses whose effectiveness is enhanced by the selection of preferred adjuvants. Novel culture methods are also employed to increase reproducible yield of cultured viruses. Live vaccines are also provided from the Calaf14 PEDV isolate.

### CROSS-REFERENCE TO RELATED APPLICATIONS

The present application is a continuation of U.S. application Ser. No. 16/282,953 filed on Feb. 22 2019, now allowed, which is a continuation of U.S. application Ser. No. 15/324,908, filed Jan. 9, 2017, now issued as U.S. Pat. No. 10,251,950, which represents the U.S. national stage (37 USC 371) of international application PCT/US2015/039475, filed Jul. 8, 2015, and claims the benefit of U.S. Provisional Applications 62/023,302 filed Jul. 11, 2014; 62/037,403 filed Aug. 14, 2014; 62/046,256 filed Sep. 5, 2014; 62/093,657 filed Dec. 18, 2014; 62/102,712 filed Jan. 13, 2015; 62/115,806 filed Feb. 13, 2015; 62/121,193 filed Feb. 26, 2015; and 62/143,412 filed Apr. 6, 2015.

### FIELD OF THE INVENTION

The present invention is directed to novel immunogenic compositions that protect swine from disease caused by porcine epidemic diarrhea virus (PEDV). The present invention is also directed to novel immunogenic compositions that protect swine from disease caused by porcine deltacoronavirus (PDCoV), and combination vaccines providing both PDCoV and PEDV antigens.

### BACKGROUND OF THE INVENTION

Porcine epidemic diarrhea (PED) is highly contagious and is characterized by dehydration, diarrhea, and high mortality in swine, particularly young piglets. The causative agent, porcine epidemic diarrhea virus (PEDV), is a single stranded, positive sense RNA virus identified to the Alphacoronoavirus genus of the family Coronaviridae. PEDV has a total genome size of approximately 28kb and contains 7 open reading frames. Symptoms of PEDV infection are often similar to those caused by transmlssible gastroenteritis virus (TGEV), also a member of the Coronaviridae. It should be noted that cross protection between PEDV and TGEV is not generally observed, the overall viral nucleotide sequences being at most about 60% similar.

PED was likely first observed in Europe circa 1970, and the causative virus was subsequently characterized (see for example M. Pensaert et al. Arch. Virol, v. 58, pp 243-247, 1978 and D. Chasey et al., Res. Vet Sci, v. 25, pp 255-256, 1978). PED disease was generally considered unknown in North America until 2013, at which point widespread outbreaks commenced, and severe economic losses to the swine industry resulted.

Prototype North American isolates have remained genetically closely related (i.e. with overall nucleotide identity generally over 99%), and are similar to Asian strains characterized there within a few years prior to the North American outbreaks. PEDV generally grows poorly in culture, and there is a need to identify both particular strains and culture conditions that are appropriate for the culturing of sufficient virus for commercial vaccine preparation.

Additionally, there is a need to develop vaccines that provide effective cross protection against known isolates of PEDV, and which are expected to provide effective cross protection against evolving, non-prototype PEDV strains.

Additionally, variant strains of PEDV (for example Calaf14, see SEQ ID NOS 1, 4 for S protein sequence) have been recently identified in Europe, which are recognizably different from known European strains. Such variant strains (similar to Calaf14 based on spike protein sequence) have also appeared in North America, and previously in Asia, and may be more similar to each other than to prototype strains. Accordingly, there is a need to identity both vaccine strains and appropriate vaccine compositions that will be effective against current and emerging worldwide outbreaks of PEDV, thus providing needed cross protection.

Porcine deltacoronavirus (PDCoV) is a member of a novel group of coronaviruses which were initially identified as “Group 3c coronaviruses” by Woo et al. (J Virol., 83(2):908-917, 2009) in various avian species. Subsequently, these viruses were reclassified as “deltacoronaviruses”, and have been identified in other avian species, as well as in pigs (Woo et al., J Virol., 86(7):3995-4007, 2012; Marthaler et al., Genome Announc., 2(2):e00278-14, 2014; Li et al., Genome Announc., 2(2):e00278-14, 2014; Wang et al., Genome Announc., 2(2):e00291-14, 2014; Wang et al., Emerg. Infect. Dis., 20(7):1227-1230, 2014). The genome size of deltacoronaviruses (˜25-26 kb) is smaller in size than PEDV and other alphacoronaviruses, which can approach 32 kb.

PDCoV has to date been detected at least in Hong Kong, Canada, China and the US, and while the death rate in piglets reported for PDCoV infections (30-40%) is apparently lower than that typically observed with PEDV infection, interpretation of field data is often difficult since co-infections with PEDV and other intestinal pathogens are common (EFSA Journal, 12(10):3877, 2014). While more knowledge on the pathogenesis and clinical implications of PDCoV is needed, this recently-identified virus appears to be an emerging pathogen in pigs. Thus, efficacious vaccine compositions for treating and preventing disease caused by PDCoV are desired, as are combination vaccines that prevent and/or treat both PEDV and PDCoV diseases.

### SUMMARY OF THE INVENTION

The present invention encompasses an immunogenic composition comprising inactivated PEDV, one or more adjuvants, and optionally one or more excipients, in an amount effective to elicit production of neutralizing antibodies in swine. The adjuvant preferably provides an oil-in-water emulsion with additional components. The immunogenic compositions of the invention protect swine from infection by PEDV, and are effective in single doses, in two-dose programs, or in vaccination programs involving multiple doses, which may be spread apart by at least a week, and optionally at greater intervals of time, such as one to several months. It should be noted that depending on the level of epidemic threat in a particular swine population, the vaccine dose program of one, two, or multiple doses may be repeated, from time to time, as a precautionary measure. Additionally, it should be noted that vaccinating a mother sow during pregnancy will provide protection to a young piglet, via maternal transfer of antibodies and T-cells in milk, although such protection may need to be followed up with additional vaccination doses to the piglet. Vaccination of all swine including piglets and adults is contemplated.

It should be noted that although the prototype North American PEDV strains used in the practice of the invention are useful in control of North American disease outbreaks (and indeed USA/Colorado/2013, see below, has now been licensed for this purpose), it has been surprisingly discovered that such prototype North American strain vaccines are also cross protective against European and Asian strains generally, and are also effective against emerging isolates of PEDV disease, such as those that appear similar to Calaf14 (and other emerging European, Asian and North American strains) based on spike sequence. One example of such an emerging North American “Calaf14-like” strain is PEDV-INDEL (OH851) first isolated by the Ohio Department of Agriculture (L. Wang et al., Emerg. Infect. Dis., 2014, v. 20, pp. 917-919). Indeed, it appears that circulating North American strains now cluster into 2 distinct clades, the recently emerging clade having insertions and deletions in spike gene (S-INDELS) which all share 98-100% identity at a nucleotide level (spike gene), but such recent isolates only present about 96-97% identity at the nucleotide level (spike gene) with initial (prototype) North American strains (see also A. Vlasova et al. “Distinct Characteristics and Complex Evolution of PEDV Strains, North America, May 2013-February 2014”, Emerging Infectious Disease, Vol 20, No. 10, 2014. Such S-INDELs tend to be less virulent, and more readily attenuated for use in live vaccines. The first public disclosure of North American S-INDELs may be that of the Iowa State University Veterinary Diagnostic Laboratory, on Jan. 30, 2014, defined as having only 93.9-94.6% identity to previously identified USA strains, but being nearly identical (99+%) to each other. Useful insertions and deletions need not be confined to the spike gene. ORF3 modifications (particularly deletions) have been correlated with adaptation to cell culture and reduction of pathogenicity (see S-J. Park etal., Virus Genes, 2008, v 36, pp. 95-104; and others (see J. Zhang et al. Journal of Clinical Microbiology, v. 52(9), pp. 3511-3514, 2014) have commented that classification of PEDVs based on ORF3 may be appropriate. INDEL-type strains have also been previously identified in Asia. see for example, D. S. Song et al., Research in Veterinary Science, v 82, pp. 134-140, 2007; S-J Park et al., Virus Genes, v 35, pp..55-64, 2007; and further discussion thereof by D. Song et al. (Virus Genes (2012) v 44 pp. 167-175) referring to the DR13 strain, passaged to level 100, and previously licensed in Korea (see also KR patent 0502008). Finally T. Oka et al., Veterinary Microbiology, 173, pp 258-269 (2014) disclose additional S-INDEL strains, and a PEDV strain related to prototype virulent strains but bearing a large 197 amino acid deletion from the S protein, possibly resulting from passaging.

Thus, according to the practice of the present invention, there are provided vaccines against PEDV based on inactivated virus, such as inactivated USA/Colorado/2013 strain (SEQ ID NO: 7), which are highly effective, including on a worldwide basis (to include North America, Europe and Asia), including against prototype strains and INDELs. In a further important aspect of the invention, there are also provided vaccines against PEDV based on Calaf14 strain (whether inactivated or live) which are similarly worldwide effective. Thus, the vaccinating compositions of the present invention are useful to protect swine from disease or challenge by PEDV generally, on a worldwide basis, including more recent isolates, such as, but not limited to isolates that show homology with S-INDEL North American variants, such as OH851, or other emerging variants. In this regard, protection is accorded against all of the prototype, INDEL, or other variant strains as mentioned in the immediately preceding paragraph. It should also be understood that by use of preferred “TXO” adjuvant compositions (as further defined below) it is possible to provide inactivated vaccine compositions based on nearly any PEDV or PDCoV strain that are effective and protective for challenge in swine with nearly any other PEDV or PDCoV isolate.

The present invention also encompasses an immunogenic composition comprising inactivated PDCoV, one or more adjuvants, and optionally one or more excipients, in an amount effective to elicit production of neutralizing antibodies in swine. The adjuvant preferably provides an oil-in-water emulsion with additional components. The immunogenic compositions of the invention protect swine from infection by PDCoV, and are effective in single doses, in two-dose programs, or in vaccination programs involving multiple doses, which may be spread apart by at least a week, and optionally at greater intervals of time, such as one to several months.

The present invention also encompasses an immunogenic composition comprising both inactivated PEDV and inactivated PDCoV. Additionally, the immunogenic composition can comprise other swine antigens, including Escherichia coli and Clostridium perfringens, types A-D, the dosages of which would be equivalent to those found in the commercially-available vaccines, Gletvax® and Litterguard®. The vaccines can contain one or more adjuvants, and optionally one or more excipients, in an amount effective to elicit production of neutralizing antibodies in swine. The adjuvant preferably provides an oil-in-water emulsion with additional components. The immunogenic compositions of the invention protect swine from infection by both PEDV and PDCoV, and are effective in single doses, in two-dose programs, or in vaccination programs involving multiple doses, which may be spread apart by at least a week, and optionally at greater intervals of time, such as one to several months.

### BRIEF DESCRIPTION OF THE FIGURES

FIGS. 1-3 depict certain aspects of optimized passaging of PEDV in Vero 76 cells based on detection of morphology of infected cells (USA/Colorado/2013 strain, SEQ ID NO: 7).

FIG. 1 shows PEDV-infected Vero cells with “bubble effect” caused by the virus.

FIG. 2 shows PEDV-infected Vero cells that evidence a surrounding “filmy layer”.

FIG. 3 shows non-infected Vero cells, instead showing the effect of high trypsin concentration, but without PEDV infection.

FIG. 4 shows the nucleotide sequence for recent Spanish isolate Calaf14 corresponding to the spike protein (SEQ ID NO: 1)

FIG. 5 shows a comparison of amino acid sequence percent identities (spike protein) for various European and North American isolates.

FIG. 6 shows a phylogenetic tree of numerous known PEDV isolates based on spike protein, as identified by their depository record locators.

FIG. 7 provides an identity scores table of complete encoding sequences of spike protein for three European PEDV isolates, CV777, Br1-87, and Calaf14.

FIG. 8 provides an identity scores table of complete spike protein amino acid for three European PEDV isolates, CV777, Br1-87 and Calaf14.

FIGS. 9A-9E show full amino acid sequence alignments for full length spike (S) proteins for European strains CV777, BR1-87 and Calaf14. (SEQ ID NOS: 6, 5 and 4, respectively). Starting at the amino terminus, corresponding to FIG. 9A, shown is, consecutively, amino acid sequence ending at, respectively, residues 250, 550, 850, 1150, then ending approximately at position 1383.

FIGS. 10A-10R show full encoding nucleotide sequence alignments for full length spike (S) proteins for European strains CV777, BR1-87 and Calaf14 (SEQ ID NOS: 3, 2 and 1, respectively). Starting at the amino terminus, corresponding to FIG. 10A, shown is, consecutively, nucleic acid residue sequence ending at, respectively, residues 240, 480, 720, 960, 1200, 1440, 1680, 1920, 2160, 2400, 2640, 3120, 3360, 3600, 3840, 4080, then ending approximately at position 4140.

### BRIEF DESCRIPTION OF THE SEQUENCE LISTING

SEQ ID NO: 1 provides, as a DNA version, the nucleotide sequence encoding for the spike protein of PEDV strain Calaf14.

SEQ ID NO: 2 provides, as a DNA version, the nucleotide sequence encoding for the spike protein of PEDV strain Br1-87.

SEQ ID NO: 3 provides, as a DNA version, the nucleotide sequence encoding for the spike protein of PEDV strain CV777.

SEQ ID NO: 4 provides the amino acid sequence of spike protein of PEVD strain Calaf14.

SEQ ID NO: 5 provides the amino acid sequence of spike protein of PEVD strain Br1-87.

SEQ ID NO: 6 provides the amino acid sequence of spike protein of PEVD strain CV777.

SEQ ID NO: 7 provides, as a DNA version, the full nucleotide sequence encoding for the USA/Colorado/2013 PEDV virus.

SEQ ID NOS: 8-10 provide the nucleotide sequence of oligonucleotides used in cloning processes.

SEQ ID NO: 11 provides, as a DNA version, the full nucleotide sequence encoding for the USA/Indiana/2014/8501010 PDCoV virus.

SEQ ID NO: 12 provides, as a DNA version, the full nucleotide sequence encoding for the NVSL USA/Michigan/8977/2014 PDCoV virus.

### DETAILED DESCRIPTION OF THE INVENTION

The present invention provides novel and efficacious vaccines useful to preventing disease caused by PEVD and PDCoV.

### Definitions

Vaccines can be made more efficacious by including an appropriate adjuvant in the composition. The term “adjuvant” generally refers to any material that increases the humoral or cellular immune response to an antigen. Adjuvants are used to accomplish two objectives: They slow the release of antigens from the injection site, and they enhance stimulation of the immune system. Traditional vaccines are generally composed of a crude preparation of inactivated or killed or modified live pathogenic microorganisms. The impurities associated with these cultures of pathological microorganisms may act as an adjuvant to enhance the immune response. However, the immunity invoked by vaccines that use homogeneous preparations of pathological microorganisms or purified protein subunits as antigens is often poor. The addition of certain exogenous materials such as an adjuvant therefore becomes necessary. Further, in some cases, synthetic and subunit vaccines may be expensive to produce. Also, in some cases, the pathogen cannot be grown on a commercial scale, and thus, synthetic/subunit vaccines represent the only viable option. The addition of an adjuvant may permit the use of a smaller dose of antigen to stimulate a similar immune response, thereby reducing the production cost of the vaccine. Thus, the effectiveness of some injectable medicinal agents may be significantly increased when the agent is combined with an adjuvant.

Many factors must be taken into consideration in the selection of an adjuvant. An adjuvant should cause a relatively slow rate of release and absorption of the antigen in an efficient manner with minimum toxic, allergenic, irritating, and other undesirable effects to the host. To be desirable, an adjuvant should be non-viricidal, biodegradable, capable of consistently creating a high level of immunity, capable of stimulating cross protection, compatible with multiple antigens, efficacious in multiple species, non-toxic, and safe for the host (eg, no injection site reactions). Other desirable characteristics of an adjuvant are that it is capable of micro-dosing, is dose sparing, has excellent shelf stability, is amenable to drying, can be made oil-free, can exist as either a solid or a liquid, is isotonic, is easily manufactured, and is inexpensive to produce. Finally, it is highly desirable for an adjuvant to be configurable so as to induce either a humoral or cellular immune response or both, depending on the requirements of the vaccination scenario. However, the number of adjuvants that can meet the above requirements is limited. The choice of an adjuvant depends upon the needs for the vaccine, whether it be an increase in the magnitude or function of the antibody response, an increase in cell mediated immune response, an induction of mucosal immunity, or a reduction in antigen dose. A number of adjuvants have been proposed, however, none has been shown to be ideally suited for all vaccines. The first adjuvant reported in the literature was Freund's Complete Adjuvant (FCA) which contains a water-in-oil emulsion and extracts of mycobacterium. Unfortunately, FCA is poorly tolerated and it can cause uncontrolled inflammation. Since the discovery of FCA over 80 years ago efforts have been made to reduce the unwanted side effects of adjuvants.

Some other materials that have been used as adjuvants include metallic oxides (e.g., aluminum hydroxide), alum, inorganic chelates of salts, gelatins, various paraffin-type oils, synthesized resins, alginates, mucoid and polysaccharide compounds, caseinates, and blood-derived substances such as fibrin clots. While these materials are generally efficacious at stimulating the immune system, none has been found to be entirely satisfactory due to adverse effects in the host (e.g., production of sterile abcesses, organ damage, carcinogenicity, or allergenic responses) or undesirable pharmaceutical properties (e.g., rapid dispersion or poor control of dispersion from the injection site, or swelling of the material).

“Cellular immune response” or “cell mediated immune response” is one mediated by T-lymphocytes or other white blood cells or both, and includes the production of cytokines, chemokines and similar molecules produced by activated T-cells, white blood cells, or both; or a T lymphocyte or other immune cell response that kills an infected cell.

The term “emulsifier” is used broadly in the instant disclosure. It includes substances generally accepted as emulsifiers, e.g., different products of TWEEN® or SPAN® product lines (fatty acid esters of polyethoxylated sorbitol and fatty-acid-substituted sorbitan surfactants, respectively), and different solubility enhancers such as PEG-40 Castor Oil or another PEGylated hydrogenated oil.

“Humoral immune response” refers to one that is mediated by antibodies. “Immune response” in a subject refers to the development of a humoral immune response, a cellular immune response, or a humoral and a cellular immune response to an antigen. Immune responses can usually be determined using standard immunoassays and neutralization assays, which are known in the art.

“Immunologically protective amount” or “immunologically effective amount” or “effective amount to produce an immune response” of an antigen is an amount effective to induce an immunogenic response in the recipient. The immunogenic response may be sufficient for diagnostic purposes or other testing, or may be adequate to prevent signs or symptoms of disease, including adverse health effects or complications thereof, caused by infection with a disease agent. Either humoral immunity or cell-mediated immunity or both may be induced. The immunogenic response of an animal to an immunogenic composition may be evaluated, e.g., indirectly through measurement of antibody titers, lymphocyte proliferation assays, or directly through monitoring signs and symptoms after challenge with wild type strain, whereas the protective immunity conferred by a vaccine can be evaluated by measuring, e.g., reduction in clinical signs such as mortality, morbidity, temperature number, overall physical condition, and overall health and performance of the subject. The immune response may comprise, without limitation, induction of cellular and/or humoral immunity. “Immunogenic” means evoking an immune or antigenic response. Thus an immunogenic composition would be any composition that induces an immune response.

“Therapeutically effective amount” refers to an amount of an antigen or vaccine that would induce an immune response in a subject receiving the antigen or vaccine which is adequate to prevent or reduce signs or symptoms of disease, including adverse health effects or complications thereof, caused by infection with a pathogen, such as a virus or a bacterium. Humoral immunity or cell-mediated immunity or both humoral and cell-mediated immunity may be induced. The immunogenic response of an animal to a vaccine may be evaluated, e.g., indirectly through measurement of antibody titers, lymphocyte proliferation assays, or directly through monitoring signs and symptoms after challenge with wild type strain. The protective immunity conferred by a vaccine can be evaluated by measuring, e.g., reduction in clinical signs such as mortality, morbidity, temperature number, overall physical condition, and overall health and performance of the subject. The amount of a vaccine that is therapeutically effective may vary depending on the particular adjuvant used, the particular antigen used, or the condition of the subject, and can be determined by one skilled in the art.

“TCID₅₀” refers to “tissue culture infective dose” and is defined as that dilution of a virus required to infect 50% of a given batch of inoculated cell cultures. Various methods may be used to calculate TCID₅₀, including the Spearman-Karber method which is utilized throughout this specification. For a description of the Spearman-Karber method, see B. W. Mahy & H. O. Kangro, Virology Methods Manual, p. 25-46 (1996).

### Vaccine & Immunogenic Compositions

The vaccine and immunogenic composition of the present invention induces at least one of a number of humoral and cellular immune responses in a subject swine that has been administered a vaccine composition of the invention. Generally, the vaccine compositions of the invention may be administered to swine of any age, whether male or female, irrespective of reproductive status, and although it is contemplated that a two-dose regimen will be most common, single dose and multiple dose vaccine treatments are also effective in the practice of the invention. A most preferred virus for use according to all aspects of the invention relating to PEDV is USA/Colorado/2013, whose sequence is deposited as GenBank accession No. KF272920, of the NCBI of the United States National Institutes of Health. Bethesda, MD (see SEQ ID NO:7 for encoding sequence as DNA).

A further preferred virus is Calaf14, as further discussed below (see SEQ ID NO: 1,4). Most preferred are viruses encoded from polynucleotide sequence having 99.0, 99.5, and 99.9% identity to the full encoding sequence for Calaf14 or the spike gene thereof.

A preferred virus for use according to all aspects of the invention relating to PDCoV is USA/Michigan/8977/2014, whose sequence is deposited as GenBank accession No. KM012168 (see SEQ ID NO: 12 for encoding sequence as DNA). Another preferred virus for use according to all aspects of the invention relating to PDCoV is USA/Indiana/2014/8501010 (see SEQ ID NO: 11 for encoding sequence as DNA).

GenBank® is the recognized US-NIH genetic sequence database, comprising an annotated collection of publicly available DNA sequences, and which further incorporates submlssions from the European Molecular Biology Laboratory (EMBL) and the DNA DataBank of Japan (DDBJ), see Nucleic Acids Research, January 2013,v 41(D1) D36-42 for discussion.

### Viral Isolates

The adjuvanted vaccine compositions of the invention effectively incorporate all recognized strains or isolates of PEDV, including strains isolated from Europe, Asia and North America, including preferably all strains that have at least about 80% overall nucleotide identity to North American strain USA/Colorado/2013, deposited as GenBank accession No. KF272920 (see SEQ ID NO:7 for seed stock therefrom, shown as DNA copy). Preferably, the overall nucleotide homology is 70%, 80%, 90%, 95%, 96%, 97%, 98% or 99% or greater to USA/Colorado/2013, more preferably at least 95% or higher. Accordingly, additional representative strains useful in the practice of all aspects of the invention include, without limitation, strain SDCV/USA/Illinois121/2014; strain USA/Colorado/2013 deposited as GenBank accession No. KF272920; Chinese strain AH2012, deposited as GenBank accession No. KC210145; strain 13-019349, deposited as GenBank accession No. KF267450; strain CH-ZMDZY-11 deposited as GenBank accession No. KC196276; strain OH851 (Ohio); European strain CV777 (see R. Kocherhans et al., Virus Genes, vol 23(2), pp 137-144, 2001; and strains IA2013-KF452322 and IN2013-KF452323 (see G. Stevenson et al. J. Vet. Diagn. Invest., vol 25, pp. 649-654, 2013. Use of strain USA/Colorado/2013 deposited as GenBank accession No. KF272920 is preferred. Additional preferred strains, useful in the practice of all aspects of the invention, all being about 99% or higher identical to USA/Colorado/2013 deposited as GenBank Accession No. KF272920, include: GenBank Accessions KJ645688 (USA/lowa96/2013); KJ645640 (USA/Oklahoma32/2013); KJ778615 (NPL-PEDv/2013); KJ645647 (USA/Minnesota4l/2013); KJ645637 ((USA/Kansas29/2013); KJ645639 (USA/Texas31/2013); KJ645666 (USA/lowa70/2013); KJ645646 (USA/NorthCarolina40/2013); KM189367 (PEDv ON-018); and KJ645669 (USA/Wisconsin74/2013).

According to the practice of the invention, isolates of PEDV useful in the manufacture of adjuvanted vaccines may also be compared to USA/Colorado/2013 (deposited as GenBank accession No. KF272920) on the basis of spike protein amino acid sequence. Those viral isolates having spike protein sequences that are at least 70%, 80%, 90%, 95%, 96%, 97%, 98% and 99% identical to that provided by KF272920, most preferably 95% or higher, are preferred in the practice of all aspects of the invention. Taking into account that AID56763 represents the GenBank (US NIH/NCBI) Accession number for the spike protein sequence encoded within KF272920, the following PEDV isolates (as identified by their spike protein accessions) are among the reported virus strains or isolates that are most preferred for use in all aspects of the present invention: AID56757.1; AHA38139.1; AGO58924.1; AHA38125.1; AIM47748.1; AID56895.1: AID5669.1: A1120255.1: AGG34694.1; AIE15986.1; AHG05730.1; AHG05733.1 (all being representative of those having above 99% identity to the USA/Colorado/2013 spike sequence), and further, AIC82397.1; AFL02631.1; AHB33810.1; AFQ37598.1; AGG34691.1; AFJ97030.1; AFR11479.1; and AEW22948.1 (all being representative of those having above 98% identity to the USA/Colorado/2013 spike sequence). As noted, the USA-PEDV isolate shown by complete nucleotide sequence as SEQ ID NO:7 is highly preferred as a vaccine for all aspects of the practice of the present invention.

Typically, in the case of adjuvanted vaccines, the virus component is killed, however those skilled in the art will recognize that certain adjuvants are compatible with a live virus vaccine.

It is also generally recognized that evolving strains of PEDV, such as INDELs, are often naturally attenuated compared to older prototype strains, and thus may be used as vaccines wherein the virus is live attenuated, or inactivated. Calaf14 is an example of such strains, where only minimal further passaging may be needed to provide a safe vaccine attenuate. Exemplary vaccine viruses of the invention therefor also include those that have 95, 96, 97, 98, 99 and most preferably 99.5% or higher sequence identify with Calaf14, whether measured amino acid or encoding nucleotide sequence, for the spike protein or based on the full viral sequence.

Besides the various PEDV strains that may be used in an adjuvanted vaccine, recombinant spike protein, including the S1 and/or S2 fragments thereof, may also be used in a vaccine. Spike protein or S1 or S2 fragments may also be employed as diagnostic antigens. Exemplary PEDV spike protein sequences include, but are not limited to, those provided as SEQ ID NOS: 4, 5 6 and as encoded from SEQ ID NO:7.

The adjuvanted vaccine compositions of the invention effectively incorporate all recognized strains or isolates of PDCoV, including strains isolated from North America, including preferably, but not necessarily limited to, all strains that have at least about 80% overall nucleotide identity to isolate KNU14-04, deposited as GenBank accession No. KM820765; isolate USA/IA/2014/8734, deposited as GenBank accession No. KJ567050; isolate HKU15 strain MI6148, deposited as GenBank accession No. KJ620016; isolate HKU15 strain MN3092, deposited as GenBank accession No. KJ584360; isolate HKU15 strain NE3579, deposited as GenBank accession No. KJ584359; isolate HKU15 strain PA3148, deposited as GenBank accession No. KJ584358; isolate HKU15 strain KY4813, deposited as GenBank accession No. KJ584357; isolate HKU15 strain SD3424, deposited as GenBank accession No. KJ584356; isolate HKU15 strain IL2768, deposited as GenBank accession No. KJ584355; isolate OhioCVM1/2014, deposited as GenBank accession No. KJ769231; isolate PDCoV/USA/Illinois121/2014, deposited as GenBank accession No. KJ481931; isolate PDCoV/USA/Ohio137/2014, deposited as GenBank accession No. KJ601780; isolate PDCoV/USA/Illinois136/2014, deposited as GenBank accession No. KJ601779; isolate PDCoV/USA/Illinois134/2014, deposited as GenBank accession No. KJ601778; isolate PDCoV/USA/Illinois133/2014, deposited as GenBank accession No. KJ601777; isolate HKU15 strain IN2847, deposited as GenBank accession No. KJ569769; isolate HKU15 strain OH1987, deposited as GenBank accession No. KJ462462; and isolate HKU15 strain HKU15-155, deposited as GenBank accession No. JQ065043.

Besides the various PDCoV strains that may be used in a vaccine, recombinant spike protein, including the S1 and/or S2 fragments, may also be used in a vaccine. Spike protein or S1 or S2 fragments may also be employed as diagnostic antigens. Exemplary spike protein sequences include, but are not limited to, those of PDCoV isolates USA/IA/2014/8734, USA/Michigan/8977/2014, and USA/Indiana/2014/8501010.

### Culturing of Virus

Isolation and propagation of PEDV has been generally difficult. Initial studies using Vero cells for propagation in culture have only been partially effective, and have required a trypsin-containing medium, often with excessive cytopathic effect including cell fusion, synctia formation, and cell detachment (see, for example K. Kusangi et al., J. Vet Med Sci, vol. 54(2), pp. 313-318, 1992, and M. Hofmann et al. J. Clinical Microbiology, vol. 26(11), pp2235-2239, 1988). Accordingly, improved passaging methods were developed for the practice of the present invention. Details of this method are provided in Examples 1 and 2 below. It should be noted that both USA/Colorado/2013 and Calaf14 can be cultured in Vero cells.

Cultivation of PDCoV has also proven not to be a straightforward process. Trypsin-containing medium is also required for propagating PDCoV; however, not all cell lines tested supported growth of the virus. Swine testicular (ST) cells have proven to support replication of SDCoV, though, and are the preferred cell line for propagation of the virus. ST cells can be obtained, for example, from the American Type Culture Collection (ATCC), Manassas, VA, USA, under deposit number CRL-1746.

### Inactivation of Virus (for Both PEDV and PDCov)

Inactivated or killed viral strains are those which have been inactivated by methods known to those skilled in the art, including treatment with formalin, betapropriolactone (BPL), binary ethyleneimine (BEI), sterilizing radiation, heat, or other such methods.

### Adjuvant Component (for Both PEDV and PDCoV)

The vaccine compositions of the invention are preferably provided as emulsions, with adjuvant components provided from a combination of lecithin in light mineral oil, and also an aluminum hydroxide component. Details concerning the composition and formulation of Amphigen® (as representative lecithin/mineral oil component) are provided in Example 5 below, as are details concerning representative aluminum hydroxide components.

According to the practice of the invention, the oil used in the adjuvant formulations of the instant invention is a light mineral oil. As used herein, the term “mineral oil” refers to a mixture of liquid hydrocarbons obtained from petrolatum via a distillation technique. The term is synonymous with “liquefied paraffin”, “liquid petrolatum” and “white mineral oil.” The term is also intended to include “light mineral oil,” i.e., oil which is similarly obtained by distillation of petrolatum, but which has a slightly lower specific gravity than white mineral oil. See, e.g., Remington's Pharmaceutical Sciences, 18th Edition (Easton, Pa.: Mack Publishing Company, 1990, at pages 788 and 1323). Mineral oil can be obtained from various commercial sources, for example, J. T. Baker (Phillipsburg, Pa.), USB Corporation (Cleveland, Ohio). Preferred mineral oil is light mineral oil commercially available under the name DRAKEOL®.

Typically, the oily phase is present in an amount from 50% to 95% by volume; preferably, in an amount of greater than 50% to 85%; more preferably, in an amount from greater than 50% to 60%, and more preferably in the amount of greater than 50-52% v/v of the vaccine composition. The oily phase includes oil and emulsifiers (e.g., SPAN® 80, TWEEN® 80 etc), if any such emulsifiers are present.

Non-natural, synthetic emulsifiers suitable for use in the adjuvant formulations of the present invention also include sorbitan-based non-ionic surfactants, e.g. fatty-acid-substituted sorbitan surfactants (commercially available under the name SPAN® or ARLACEL®), fatty acid esters of polyethoxylated sorbitol (TWEEN®), polyethylene glycol esters of fatty acids from sources such as castor oil (EMULFOR®); polyethoxylated fatty acid (e.g., stearic acid available under the name SIMULSOL® M-53), polyethoxylated isooctylphenol/formaldehyde polymer (TYLOXAPOL®), polyoxyethylene fatty alcohol ethers (BRIJ®); polyoxyethylene nonphenyl ethers (TRITON® N), polyoxyethylene isooctylphenyl ethers (TRITON® X). Preferred synthetic surfactants are the surfactants available under the name SPAN® and TWEEN®, such as TWEEN®-80 (Polyoxyethylene (20) sorbitan monooleate) and SPAN®-80 (sorbitan monooleate). Generally speaking, the emulsifier(s) may be present in the vaccine composition in an amount of 0.01% to 40% by volume, preferably, 0.1% to 15%, more preferably 2% to 10%.

In an alternative embodiment of the invention, the final vaccine composition contains SP-Oil® and Rehydragel® LV as adjuvants (or other Rehydragel® or Alhydrogel® products), with preferable amounts being about 5-20% SP-Oil (v/v) and about 5-15% Rehydragel LV (v/v), and with 5% and 12%, respectively, being most preferred amounts. In this regard it is understood that % Rehydragel refers to percent dilution from the stock commercial product. (SP-Oil @ is a fluidized oil emulsion with includes a polyoxyethylene-polyoxypropylene block copolymer (Pluronic® L121, BASF Corporation, squalene, polyoxyethylene sorbitan monooleate (Tween®80, ICI Americas) and a buffered salt solution.) In another embodiment of the invention, the final vaccine composition contains TXO as an adjuvant; TXO is generally described in WO 2015/042369. All TXO compositions disclosed therein are useful in the preparation of vaccines of the invention. In TXO, the immunostimulatory oligonucleotide (“T”), preferably an ODN, preferably containing a palindromic sequence, and optionally with a modified backbone, is present in the amount of 0.1 to 5 ug per 50 ul of the vaccine composition (e.g., 0.5-3 ug per 50 ul of the composition, or more preferably 0.09-0.11 ug per 50 ul of the composition). A preferred species thereof is SEQ ID NO: 8 as listed (page 17) in the WO2015/042369 publication. The polycationic carrier (“X”) is present in the amount of 1-20 ug per 50 ul (e.g., 3-10 ug per 50 ul, or about 5 ug per 50 ul). Light mineral oil (“0”) is also a component of the TXO adjuvant.

In certain embodiments, TXO adjuvants are prepared as follows:
a) Sorbitan monooleate, MPL-A and cholesterol are dissolved in light mineral oil. The resulting oil solution is sterile filtered;
b) The immunostimulatory oligonucleotide, Dextran DEAE and Polyoxyethylene (20) sorbitan monooleate are dissolved in aqueous phase, thus forming the aqueous solution; and
c) The aqueous solution is added to the oil solution under continuous homogenization thus forming the adjuvant formulation TXO.

It should be noted that the present invention may also be successfully practiced using wherein the adjuvant component is only Amphigen. All the adjuvant compositions of the invention can be used with any of the PEDV strains and isolates covered by the present Specification.

### Excipients (for Both PEDV and PDCov)

The immunogenic and vaccine compositions of the invention can further comprise pharmaceutically acceptable carriers, excipients and/or stabilizers (see e.g. Remington: The Science and practice of Pharmacy (2005) Lippincott Williams), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as Mercury((o-carboxyphenyl)thio)ethyl sodium salt (THIOMERSAL), octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), TWEEN or PLURONICS.

### Dosing (for Both PEDV and PDCov)

A preferred clinical indication is for treatment of both breeding sows and gilts pre-farrowing. In a preferred example (applicable to both sows and gilts), two 2-ML doses of killed vaccine will be used, the first dose being administered as early as pre-breeding to 5-weeks pre-farrowing, with the second dose administered at about 1-3 weeks pre-farrowing. Doses of killed vaccine preferably provide an amount of viral material that would correspond to a TCID₅₀ (tissue culture infective dose) of between about 10⁶ and 10⁸, more preferably between about 10⁷ and 10^{7.5}, if the virus were live, and can be varied, as is recognized in the art. Booster doses can be given two weeks prior to any subsequent farrowings. Intramuscular vaccination (all doses) is preferred, although one or more of the doses could be given subcutaneously, or less preferably, orally.

In a further preferred example, the sow or gilt is vaccinated intramuscularly at 5-weeks pre-farrowing and then 2-weeks pre-farrowing. Under these conditions (from about TCID₅₀ 10⁷ to about 10^{7.5}, a protective immune response was demonstrated in PEDV-negative vaccinated sows in that they developed antibodies (measured via fluorescent focal neutralization titer from serum samples) with neutralizing activity, and these antibodies were passively transferred to their piglets. The protocols of the invention are also applicable to the treatment of already seropositive sows and gilts, and also piglets and boars. Although it is preferred to re-vaccinate a mother sow prior to any subsequent farrowings, the vaccine compositions of the invention nonetheless can still provide protection to piglets via ongoing passive transfer of antibodies, even if the mother sow was only vaccinated in association with a previous farrowing.

It should be noted that piglets may then be vaccinated as early as Day 1 of life. For example, piglets can be vaccinated at Day 1, with a booster dose at 3 weeks of age and re-boost every 6 months, if the parent sow was not vaccinated pre-breeding; however, if the sow was vaccinated pre-breeding, and thus the piglets receives maternal antibody through colostrums, then simply boost the piglets at 3 weeks and every 6 months. Boars (typically kept for breeding purposes) should be vaccinated once every 6 months.

Variation of the dose amounts is well within the practice of the art.

### Methods of Use (for Both PDEV and PDCoV)

The invention encompasses methods of preventing PEDV virus infection comprising administering the immunogenic and vaccine compositions of the invention in a swine subject of any age.

When provided therapeutically, the vaccine is provided in an effective amount upon the detection of a symptom of actual infection. A composition is said to be “pharmacologically acceptable” if its administration can be tolerated by a recipient. Such a composition is said to be administered in a “therapeutically or prophylactically effective amount” if the amount administered is physiologically significant.

At least one vaccine or immunogenic composition of the present invention can be administered by any means that achieve the intended purpose, using a pharmaceutical composition as described herein. For example, route of administration of such a composition can be by parenteral, oral, oronasal, intranasal, intratracheal, topical, subcutaneous, intramuscular, transcutaneous, intradermal, intraperitoneal, intraocular, and intravenous administration. In one embodiment of the present invention, the composition is administered by intramuscularly. Parenteral administration can be by bolus injection or by gradual perfusion over time. Any suitable device may be used to administer the compositions, including syringes, droppers, needleless injection devices, patches, and the like. The route and device selected for use will depend on the composition of the adjuvant, the antigen, and the subject, and such are well known to the skilled artisan.

According to the present invention, an “effective amount” of a vaccine or immunogenic composition is one which is sufficient to achieve a desired biological effect, in this case at least one of cellular or humoral immune response to one or more strains of PEDV. It is understood that the effective dosage will be dependent upon the age, sex, health, and weight of the subject, kind of concurrent treatment, if any, frequency of treatment, and the nature of the effect desired. The ranges of effective doses provided below are not intended to limit the invention and represent examples of dose ranges which may be suitable for administering compositions of the present invention. However, the dosage may be tailored to the individual subject, as is understood and determinable by one of skill in the art, without undue experimentation.

### Examples

The following examples illustrate only certain and not all embodiments of the invention, and thus, should not be viewed as limiting the scope of the invention.

### Example 1: Protocol for Extraction of PEDV Virus from Tissue Samples

Approximately 1 cm of tissue was used for extraction of PEDV virus. The tissue was chopped into fine pieces using a sterile scalpel and sterile scissors in a sterile Petri dish. Work was done in a Bio-safety cabinet to ensure aseptic conditions. 2 ml of sterile PBS was added to the Petri dish to collect tissue and material was transfer to a 15 ml conical tube. Tissue was homogenized using a Qiagen TissueRuptor at 80% of maximum by pulsing for a total of 30 seconds. Homogenization was performed in an ice bucket to lessen the effect of heat on the PEDV virus. The homogenized material was filtered through a 0.45 uM filter and 60 ul of material was used for RNA isolation and PEDV Q-PCR to confirm the presence of the PEDV virus. The filtered material containing the PEDV virus was further diluted 1:10 in sterile PBS and then filtered through a 0.20 uM filter.

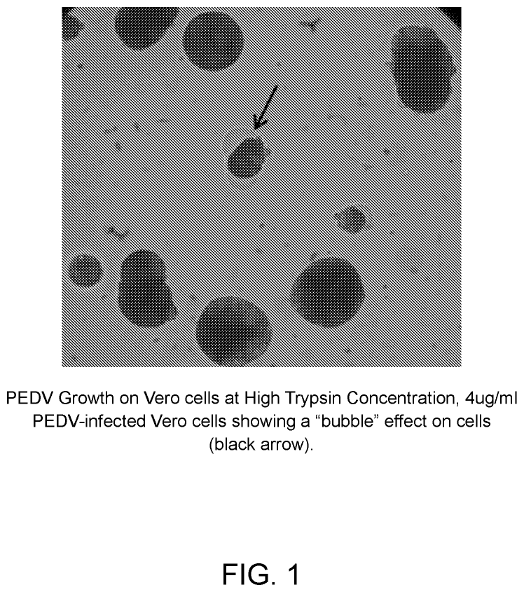

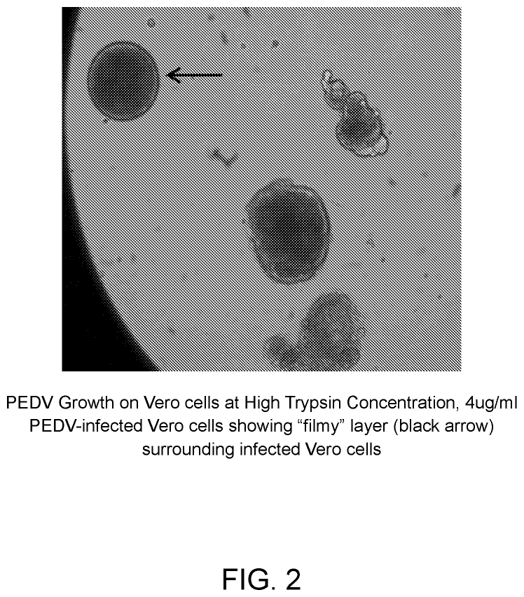

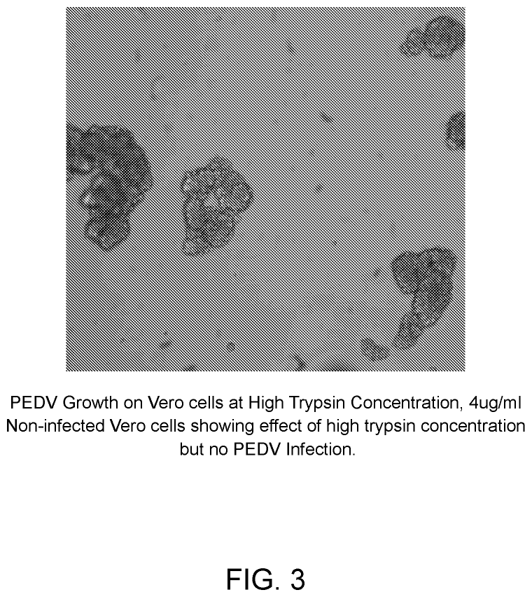

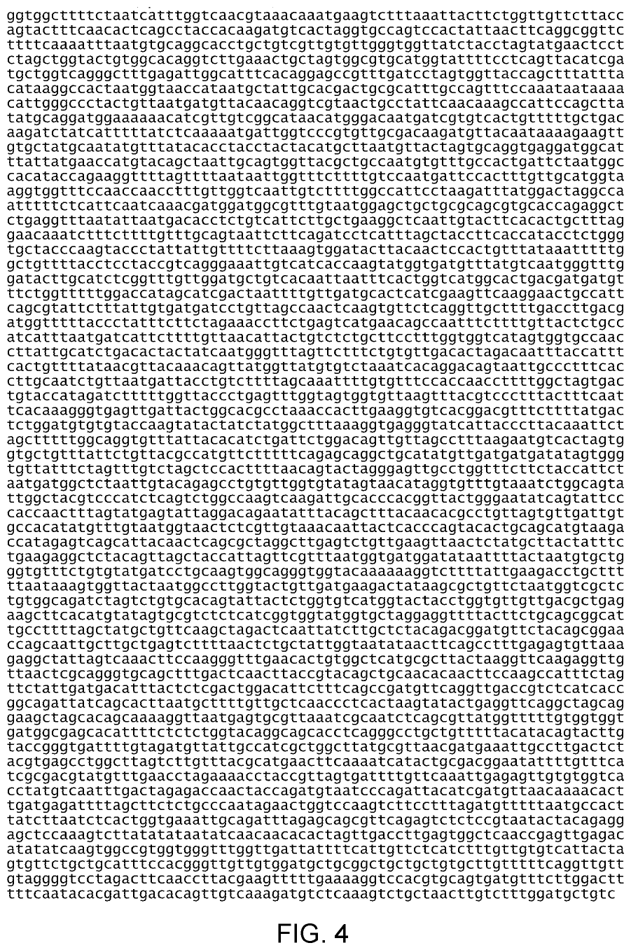

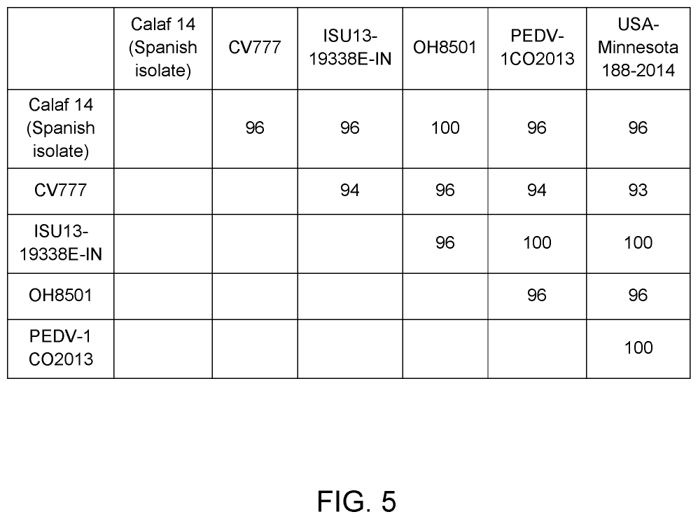

The sterile-filtered PEDV homogenate was used to infect confluent mono-layers of Vero 76 cells by transferring 1 ml of filtered material to a T-25 flask containing 2.8E+06 cells planted 3 to 4 days prior. The T-25 flasks of confluent Vero 76 cells were washed 2× with sterile PBS and 1× with DMEM media containing 10% TPB, 20 ug/ml geneticin and 4 ug/ml TPCK trypsin (equivalent to 18.8 USP units/ml). Cells were infected for 1 hour at 37° C. and 5% CO₂ in an incubator with gentle swirling every 15 minutes to ensure virus was evenly distributed to all cells. 5 ml of DMEM media containing 10% TPB, 20 ug/ml geneticin and 4 ug/ml TPCK trypsin (equivalent to 18.8 USP units/ml) was added to flasks and flask were allowed to incubate 2 days. After 2 days, flasks were frozen at −80° C. and thawed at 37° C. This material is considered as Passage 1 of the virus. One milliliter of the total volume from the flask was then used for Passage 2 of the virus. The 1 ml of Passage 1 material is used to infect a T-25 flask containing 2.8E+06 cells seeded 3 to 4 days prior. Cells were first washed 2× with sterile PBS and 1× with DMEM media containing 10% TPB, 20 ug/ml geneticin and 4 ug/ml TPCK trypsin (equivalent to 18.8 USP units/ml). Cells were infected for 1 hour at 37° C. and 5% CO₂ in an incubator with gentle swirling every 15 minutes to ensure virus was evenly distributed to cells. 5 ml of DMEM media containing 10% TPB, 20 ug/ml geneticin and 4 ug/ml TPCK trypsin (equivalent to 18.8 USP units/ml) was added to flasks and flask were allowed to incubate for 2 days. This material is Passage 2 of the PEDV virus. Passages are repeated every 2 days until the cells show signs of infection indicated by clusters of cells surrounded by a filmy layer of material and/or a bubble effect on the clustered cells (see FIGS. 1-3). The appearance of PEDV infected cells was confirmed by a decrease in Ct value in the PEDV Taqman assay. The PEDV-infected cells have a rounded up appearance with a layer of shiny film surrounding the rounded up cells.

### Example 2: Master Seed Production with Strain USA/Colorado/2013

Porcine Epidemic Diarrhea Virus Isolate PEDv-1 CO-2013 originated from a swine diagnostic specimen sourced from Colorado in 2013 and was acquired by the National Veterinary Services Laboratories in Ames, IA. (GenBank accession No. KF272920). The virus was propagated in Vero 76 cells to passage 5. The virus was then subjected to three rounds of limited dilution cloning in order to obtain a clonal population. Master seed stocks were then prepared. Extraneous agent, sterility, and Mycoplasma testing of the PEDV were conducted in accordance with 9 CFR Part 113.55, Part 113.27 and Part 113.28, respectively. The Vero cell line was designated Vero MCS Cells may be used from the MCS up to MCS+20.

For media formulation (for uninoculated cell growth medium), using a roller bottle or bioreactor production process, the cell growth medium is OPTIMEM, DMEM or equivalent cell culture media supplemented with up to 1% glutamine and 0.5 to 3% glucose, and 0.5 to 5% gamma-irradiated fetal bovine serum. Gentamicin is added at a final concentration of 20-30 μg/mL (or as determined by vaccine development experiments). For virus production medium, again for the roller bottle or bioreactor production process, the cell growth medium is OPTIMEM, OPTIPRO or equivalent supplemented with up to a 1% glutamine, >2 Units/literof 2× bovine or porcine trypsin, and 0.5 to 3% glucose. Gentamicin is added at a final concentration of 20-30 μg/mL (or as determined by vaccine development experiments). Roller bottles and bioreactors can be rinsed with cell growth medium (OPTIMEM, OPTOPRO or equivalent) up to 3×prior to infection.

### Example 3: Propagation and Harvest

Plastic flasks or roller bottles are used for growing and expanding cell cultures. Roller bottles or bioreactors will be used for virus propagation. Cells may be washed, to remove serum, prior to inoculation with virus. The virus may be diluted in virus production medium and added directly to the cell monolayer. When bioreactors are used for virus propagation, trypsinized cells will be removed from the roller bottles and a final cell passage grown in uninoculated cell growth medium. Microcarriers for the bioreactors are prepared. The seed virus is diluted to an appropriate volume within a multiplicity of infection (MOI) range of 0.0001 to 10.0

The PED virus causes observable cytopathic effect (CPE). Virus is harvested when viral-induced CPE has reached 50-100% and infected cells have begun sloughing off into the medium (cell monolayer loss exceeding 50%). The roller bottle vessels are removed from the incubator and inspected microscopically for both CPE and evidence of microbial contamination. Following the examination, the antigen fluid is harvested into appropriate sterile containers in an aseptic manner. Bioreactor fluids are examined microscopically for evidence of microbial contamination and for the presence of desired cytopathic effects (CPE). A representative seed stock result is reported as SEQ ID NO:7, as DNA)

Following examination, the viral fluids are passed through a ≤100 micron filter or stainless steel mesh screen to remove microcarriers and harvested into appropriate sterile containers in an aseptic manner. Fluids may be stored at 2° C.-7° C. for a maximum of 24 hours until inactivation. The harvested fluids may be used for seed if it is at the proper passage level and has an acceptable infectivity titer.

### Example 4: Inactivation and Neutralization

Acceptable harvested antigen production fluids will be pooled into suitable inactivation containers and inactivated using a 5 mM binary ethylenimine (BEI) solution. The mixture is cyclized for 60-80 minutes at 36±2° C. Following the addition of inactivant, the antigen will be thoroughly mixed and transferred to an inactivation vessel for the duration of the process (≥48 hours, with agitation). Neutralization of the inactivated antigen fluids will be facilitated through the addition of sterile 1 M Sodium Thiosulfate to a final concentration of approximately 20 mM-25 mM. Post-inactivated/neutralized antigen production fluids will be tested for sterility and completeness of inactivation and stored at 2-7° C. for future use in vaccine serial formulation. Genatamicin can then be used as preservative. This antibiotic will be added at the lot stage. The concentration of gentamicin in the final product will be ≤30 μg/mL.6.

### Example 5: Adjuvant Compositions and Formulation

A preferred adjuvanted vaccine composition was assembled as follows. The killed vaccine provides 7.8 log₁₀TCID₅₀ of killed USA/Colorado/2013 virus per 2 ML dose in a buffered solution further comprising about 5% (v/v) Rehydragel® (aluminum hydroxide gel) and “20% Amphigen” @ at about 25% final (v/v). Doses down to 7.0 log₁₀TCID₅₀ of killed USA/Colorado/2013 are also preferred.

Amphigen® is generally described in U.S. Pat. No. 5,084,269 and provides de-oiled lecithin (preferably soy) dissolved in a light oil, which is then dispersed into an aqueous solution or suspension of the antigen as an oil-in-water emulsion. Amphigen has been improved according to the protocols of U.S. Pat. No. 6,814,971 (see columns 8-9 thereof) to provide a so-called “20% Amphigen” component for use in the final adjuvanted vaccine compositions of the present invention. Thus, a stock mixture of 10% lecithin and 90% carrier oil (DRAKEOL®, Penreco, Karns City, PA) is diluted 1:4 with 0.63% phosphate buffered saline solution, thereby reducing the lecithin and DRAKEOL components to 2% and 18% respectively (i.e. 20% of their original concentrations). Tween 80 and Span 80 surfactants are added to the composition, with representative and preferable final amounts being 5.6% (v/v) Tween 80 and 2.4% (v/v) Span 80, wherein the Span is originally provided in the stock DRAKEOL component, and the Tween is originally provided from the buffered saline component, so that mixture of the saline and DRAKEOL components results in the finally desired surfactant concentrations. Mixture of the DRAKEOL/lecithin and saline solutions was accomplished using an In-Line Slim Emulsifier apparatus, model 405, Charles Ross and Son, Hauppauge, NY, USA.

The vaccine composition also includes Rehydragel® LV (about 2% aluminum hydroxide content in the stock material), as additional adjuvant component (available from Reheis, NJ, USA, and ChemTrade Logistics, USA). With further dilution using 0.63% PBS, the final vaccine composition contains the following compositional amounts: 7.8 log₁₀TCID₅₀ of killed USA/Colorado/2013 virus per 2 ML dose; 5% (v/v) Rehydragel® LV; 25% (v/v) of “20% Amphigen”, i.e. it is further 4-fold diluted); and 0.01% (w/v) of merthiolate.

As is understood in the art, the order of addition of components can be varied to provide the equivalent final vaccine composition. For example, an appropriate dilution of killed virus in buffer can be prepared. An appropriate amount of Rehydragel® LV (about 2% aluminum hydroxide content) stock solution can then be added, with blending, in order to permit the desired 5% (v/v) concentration of Rehydragel® LV in the actual final product. Once prepared, this intermediate stock material is combined with an appropriate amount of “20% Amphigen” stock (as generally described above, and already containing necessary amounts of Tween 80 and Span 80) to again achieve a final product having 25% (v/v) of “20% Amphigen”. An appropriate amount of 10% merthiolate can finally be added.

The vaccinate compositions of the invention permit variation in all of the ingredients, such that the total dose of antigen may be varied preferably by a factor of 100 (up or down) compared to the antigen dose stated above, and most preferably by a factor of 10 or less (up or down),. Similarly, surfactant concentrations (whether Tween or Span) may be varied by up to a factor of 10, independently of each other, or they may be deleted entirely, with replacement by appropriate concentrations of similar materials, as is well understood in the art.

Rehydragel® concentrations in the final product may be varied, first by the use of equivalent materials available from many other manufacturers (i.e. Alhydrogel®,Brenntag; Denmark), or by use of additional variations in the Rehydragel® line of products such as CG, HPA or HS. Using LV as an example, final useful concentrations thereof including from 0% to 20%, with 2-12% being more preferred, and 4-8% being most preferred, Similarly, the although the final concentration of Amphigen (expressed as % of “20% Amphigen”) is preferably 25%, this amount may vary from 5-50%, preferably 20-30% and is most preferably about 24-26%.

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. It is intended that the specification and examples be considered exemplary only with the true scope and spirit of the invention indicated by the following claims.

### Example 6: Cross Protection

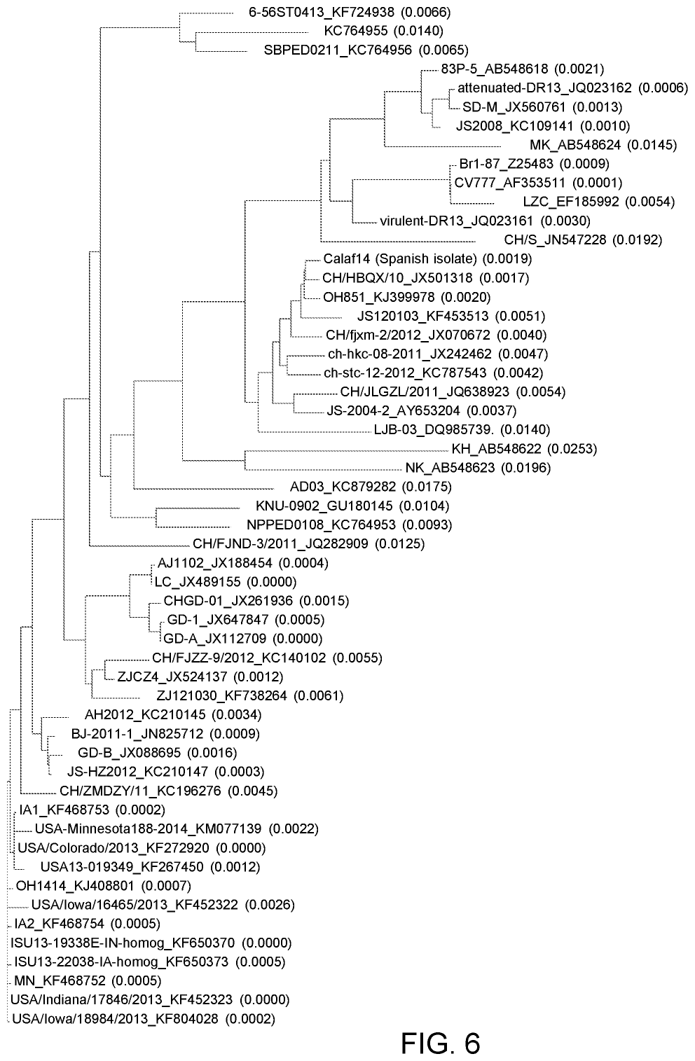

Porcine Epidemic Diarrhea virus (PEDV) was initially introduced in the United States in April 2013 and subsequently spread all over the country. Sequencing of PEDV isolates revealed similar nucleotide homology (>99%) with a Chinese strain from 2012. In Europe, several outbreaks have been reported since 2014, which are different than prior European outbreaks. The new European strains cluster with the INDEL (insertion-deletion) variants of the PEDV phylogenetic tree (FIG. 6), and warrant significant epidemiological attention.

In order to assess efficacy of an inactivated porcine epidemic diarrhea virus vaccine in pregnant sows, the following experiments were conducted. Strain USA/Colorado/2013 (deposited as GenBank accession No. KF272920) was used, and cultured and prepared as provided for above. The “Porcine Epidemic Diarrhea Vaccine, Killed Virus”, manufactured by Zoetis, is intended for pre-farrowing vaccination of sows and gilts against diarrheal disease in their neonatal pigs caused by PEDV. This vaccine was developed using a highly virulent American PEDV strain. In a preferred example, the vaccine is given intramuscularly to pregnant sows as two doses, 2 ML each, three weeks apart, at five and two weeks pre-farrowing.

The objective of the study was to determine the immunogenic efficacy of this killed vaccine, by infecting 4 day old piglets born from vaccinated pregnant sows with a new Spanish PEDV isolate (Calaf14), characteristic of recent European outbreaks, as challenge. An efficacy study of the vaccine in pregnant sows was required to evaluate the maternal antibody protection against Porcine Epidemic Diarrhea virus, since PEDV induces gastro-intestinal disease, and protection against infection and disease against PEDV is mediated by maternally-derived antibodies.

Eight pregnant sows were included in the study. At 5 weeks before farrowing, a dose (IM route) of an experimental batch of the inactivated PEDV vaccine was administered to 5 sows; 3 sows remained non-vaccinated. Three weeks later, vaccinated sows received a second dose. After farrowing, approximately at 4±1 days of age, all piglets were challenged with the Spanish PEDV strain Calaf14 (encoding nucleotides, as DNA, and amino acid sequence for spike protein thereof, are reported as SEQ ID NOS: 1 and 4 respectively), isolated from recent cases of diarrhea in neonatal pigs, and clustered with the PEDV INDEL variants. Three to four days post-challenge, all piglets were euthanized and necropsied. Twice daily after challenge, all piglets were evaluated for the presence of clinical signs, rectal temperature, body weight, and fecal swabs were taken to perform a PEDV-specific RT-qPCR. At day 3 to 4 after challenge, all piglets were euthanized, and gut tissue samples were taken.

Vaccinated sows delivered a total of 32 piglets, while control sows delivered 21 piglets. In control sows, moderate to severe diarrhea was observed in all litters, affecting 19 out of 21 piglets (90.5%). Weight loss during the study affected 12/21 piglets (57.1%), and 4 of them reached the end-point of dehydration and severe gastrointestinal clinical signs and had to be euthanized. In contrast, in vaccinated sows, 3 out of 5 litters were either non-affected by diarrhea, or only one pig in the litter was mildly affected in one single observation; in two litters, several piglets developed mild to moderate diarrhea. In total, 15 piglets born from vaccinated mothers developed diarrhea (46.9%). Weight loss was observed in only 3/32 piglets (6.5%), and none of the piglets had to be euthanized.

The clinical data obtained confirm that the Porcine Epidemic Diarrhea Vaccine, Killed Virus, manufactured by Zoetis, containing a killed US PEDV isolate as antigen, is able to confer cross-protection to piglets born from vaccinated sows, in front of the challenge with a heterologous EU PEDV isolate.

The European Challenge Virus (Spanish isolate Calaf14) was compared to two known and older European isolate on the basis of full spike protein coding sequence. The “Calaf 14” Spanish isolate was obtained from a PEDV case detected in a Spanish farm in 2014. Intestines from a 4-day-old piglet were processed to obtain a clarified intestine homogenate. RNA was extracted and the sample was found to be positive by real-time RT-PCR analysis (PEDV N gene-based real-time RT-PCR assay).

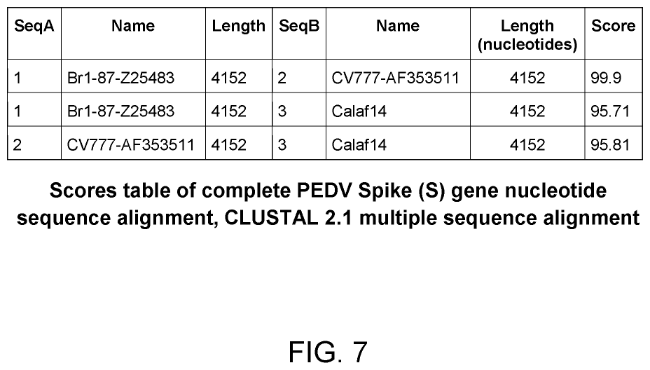

The complete spike (S) gene (4152nt) was sequenced as previously described (Chen, Q., et al. “Isolation and characterization of porcine epidemic diarrhea viruses associated with the 2013 disease outbreak among swine in the United States.” J Clin Microbiol 52(1): 234-243 2014). The complete S gene coding sequence of the Calaf14 PEDV (SEQ ID NO:1) currently circulating isolate was compared to those of the two PEDV European reference isolates (CV777, see SEQ ID NO: 3 and Br1/87, see SEQ ID NO:2) available in the GenBank (accession numbers AF353511 and Z25483 respectively). No sequences are published or available in GenBank from the most recent outbreaks occurred in other European countries. For the alignment, both Vector NTI Advance 11.5 and CLUSTAL 2.1 multiple sequence alignment were used. Analysis showed that the two European isolates were practically identical to each other (99.9% nucleotide identity, see Appendix 1). However, when compared to Calaf14 isolate identity scores decreased to 95.71% identity for Br1/87 and 95.81% identity with CV777 isolate (see FIG. 7).

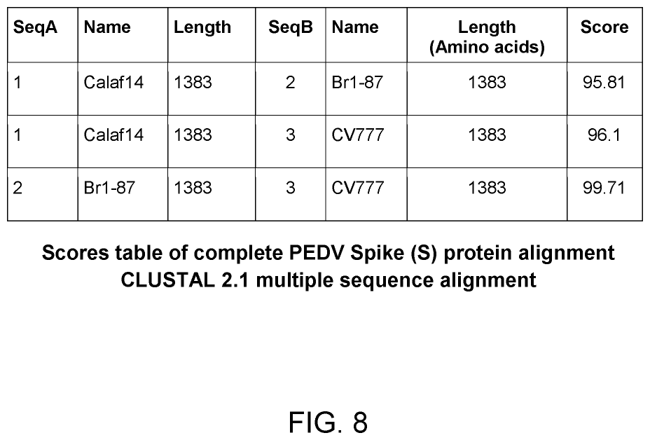

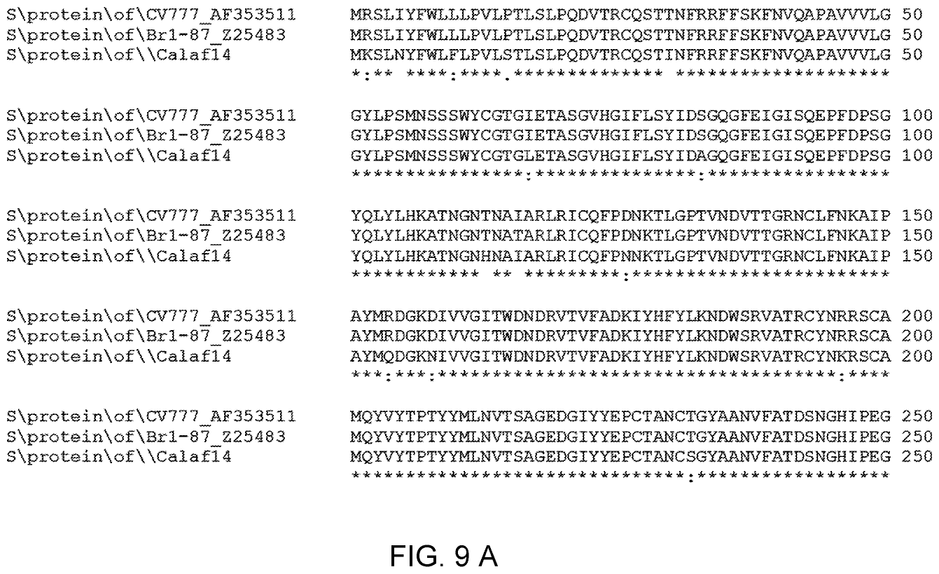

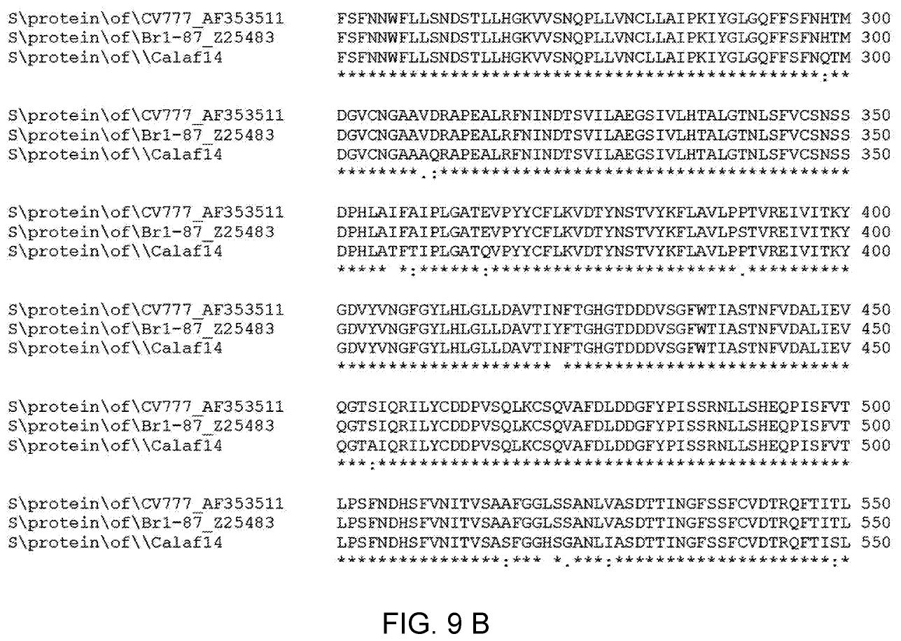

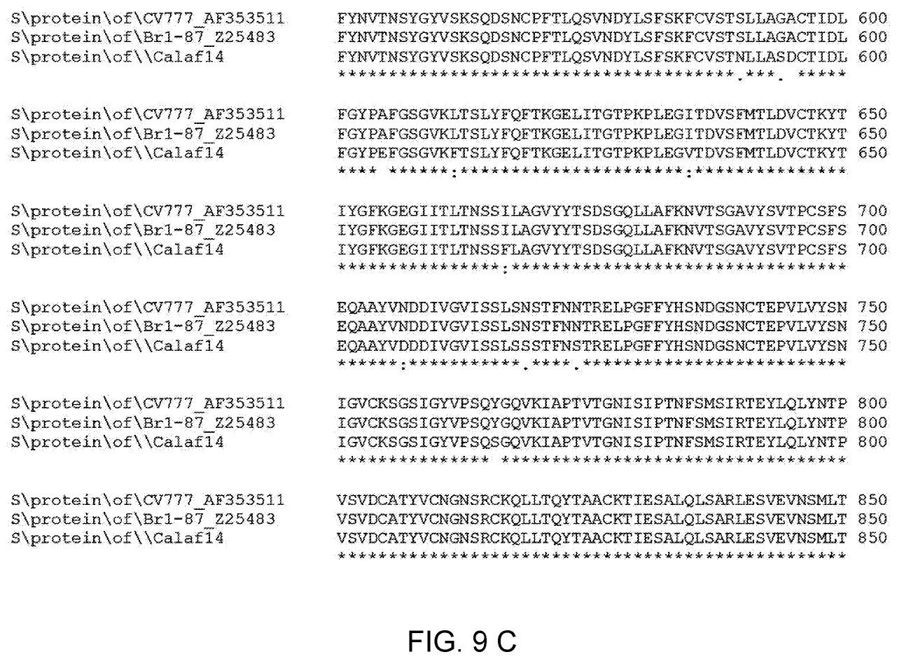

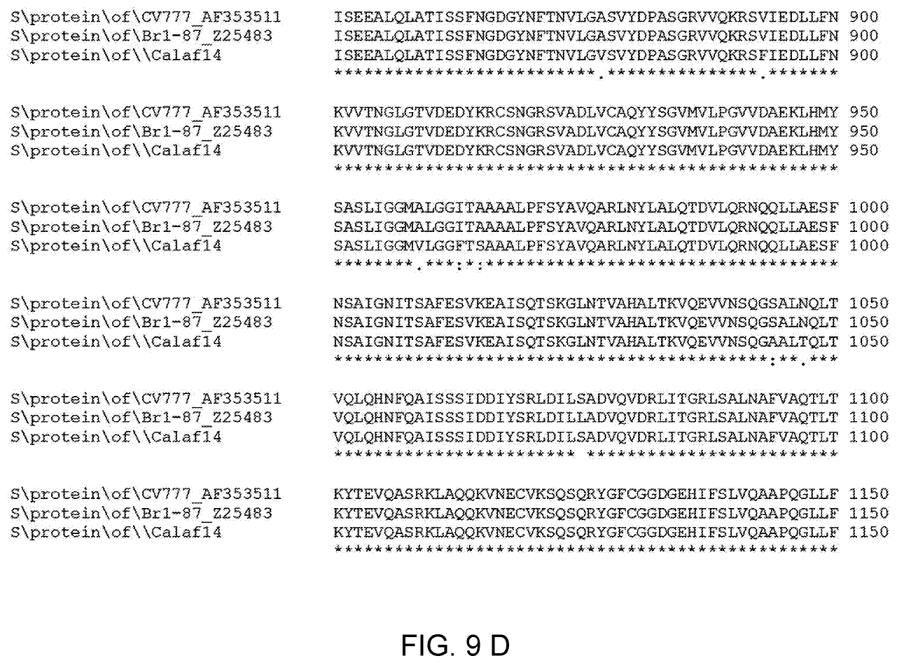

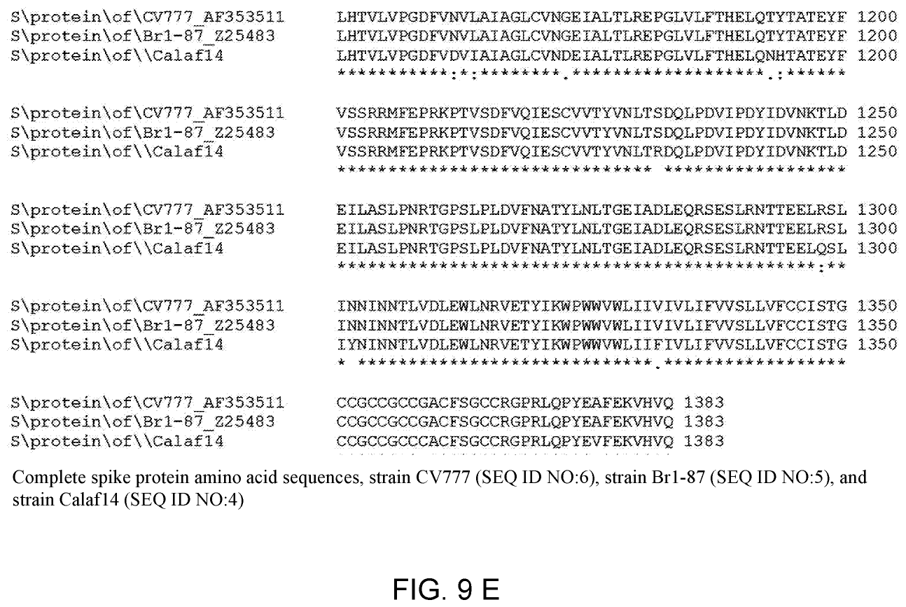

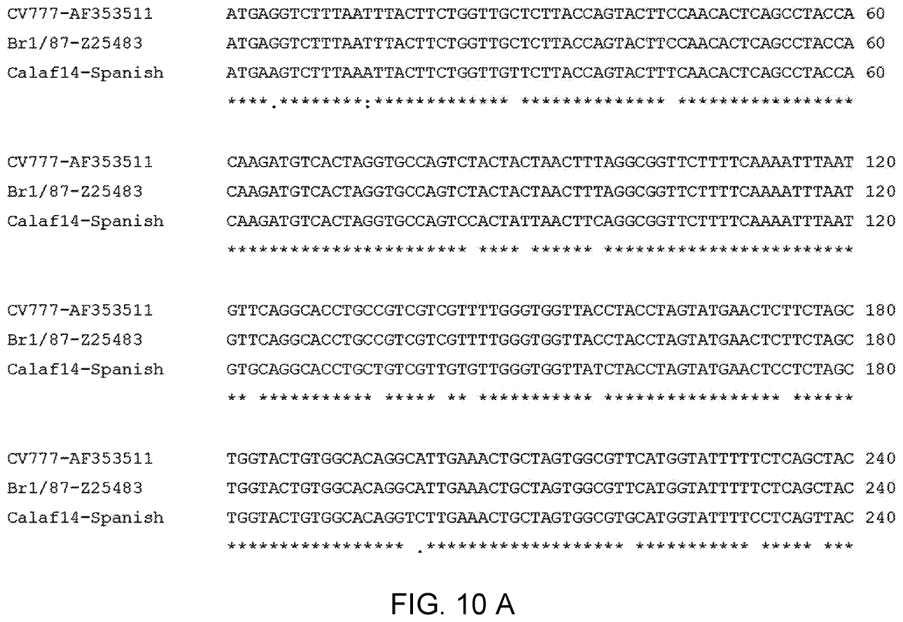

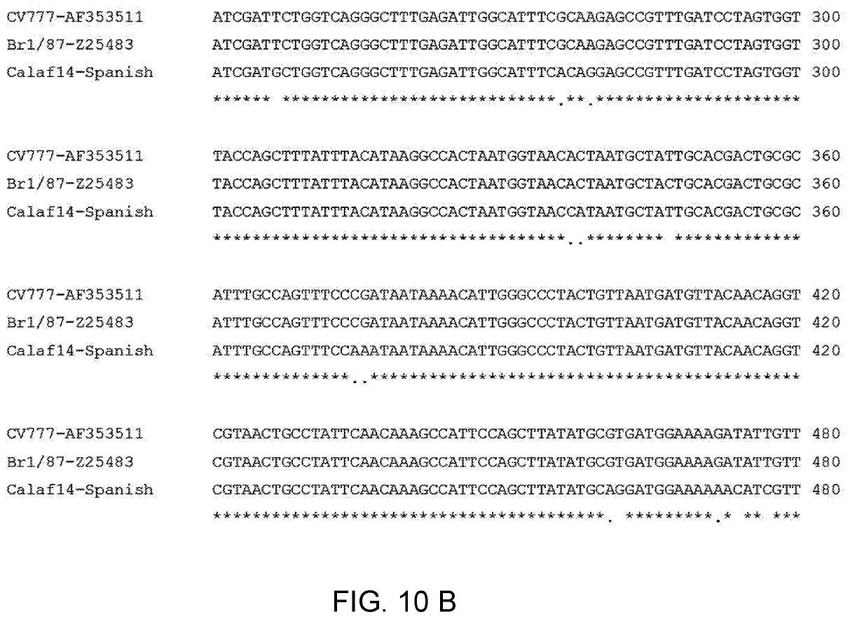

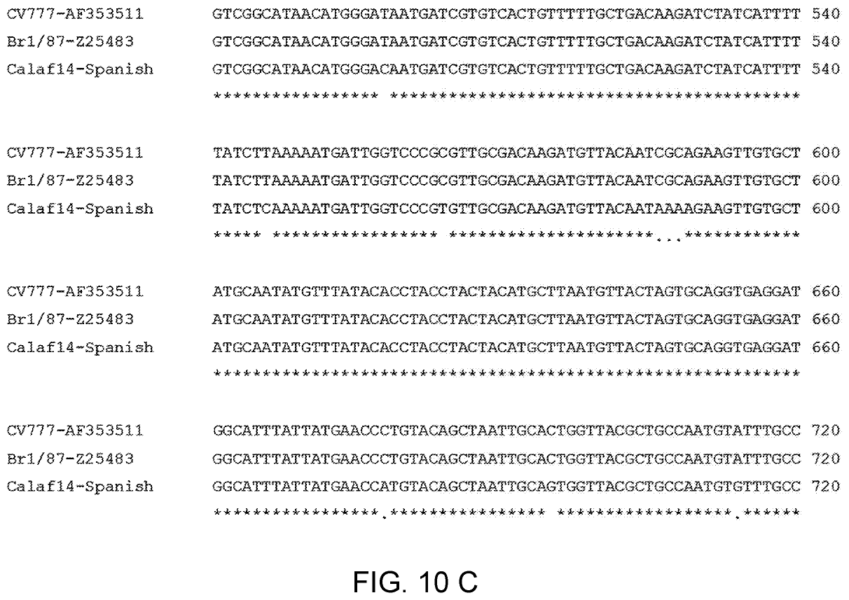

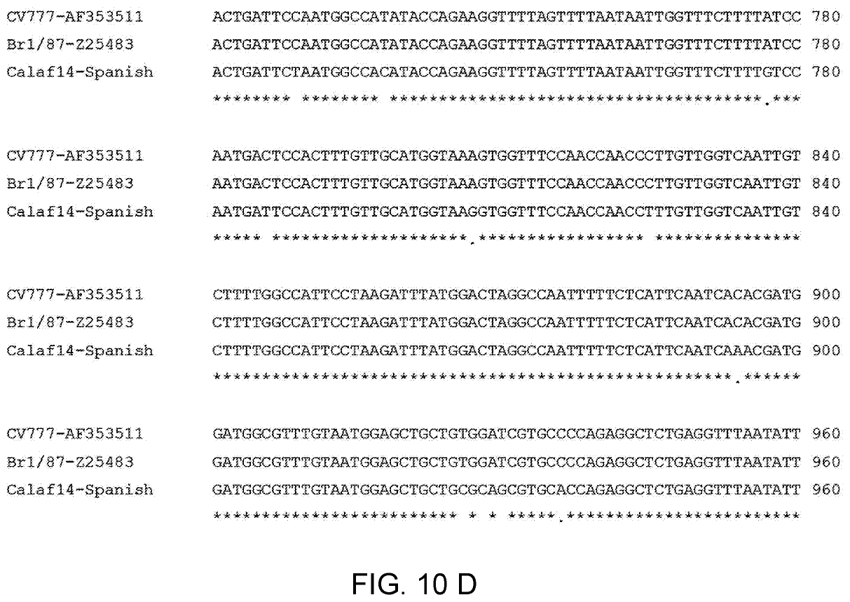

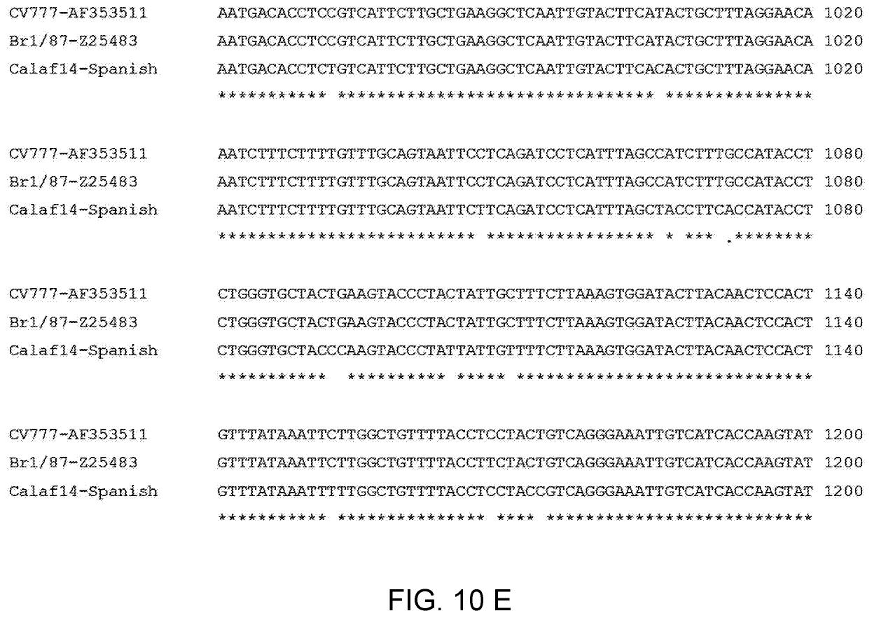

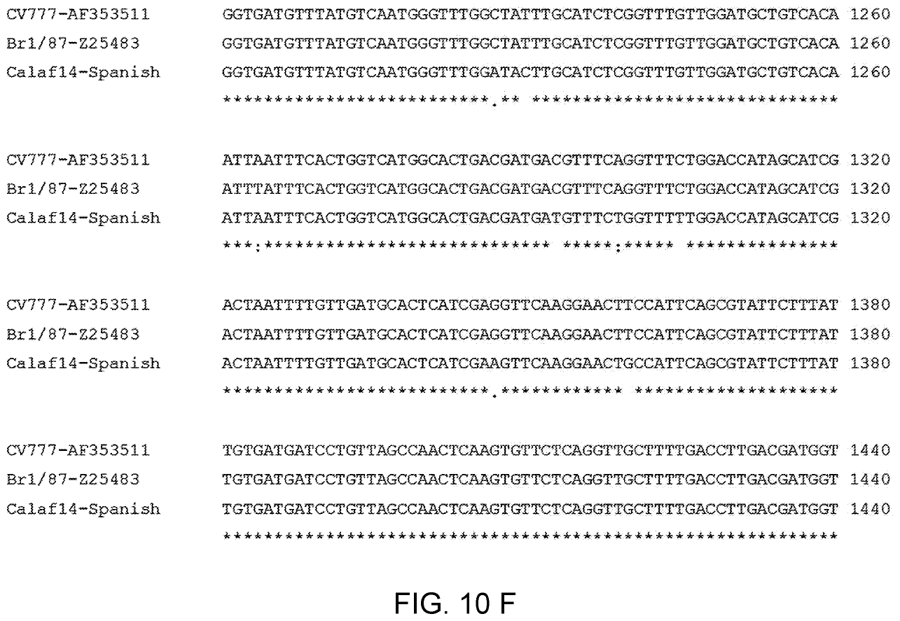

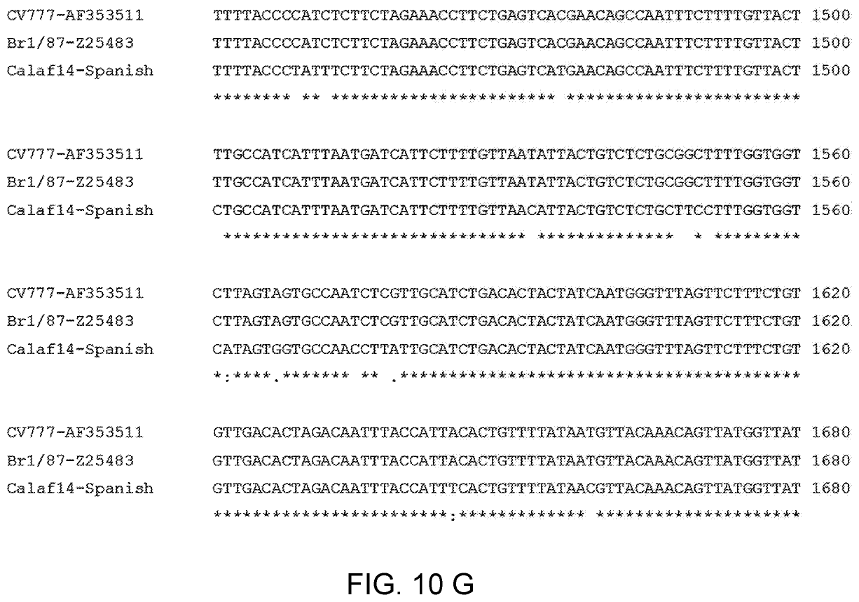

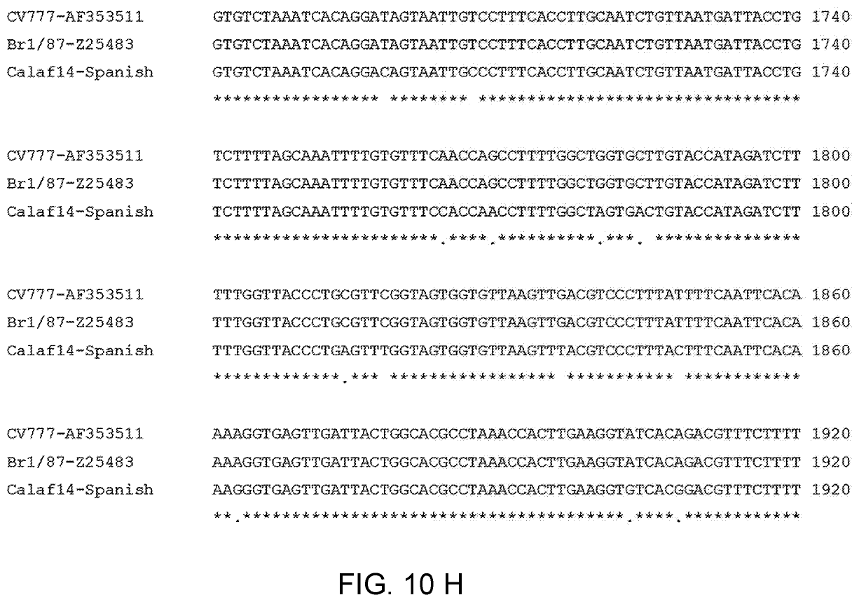

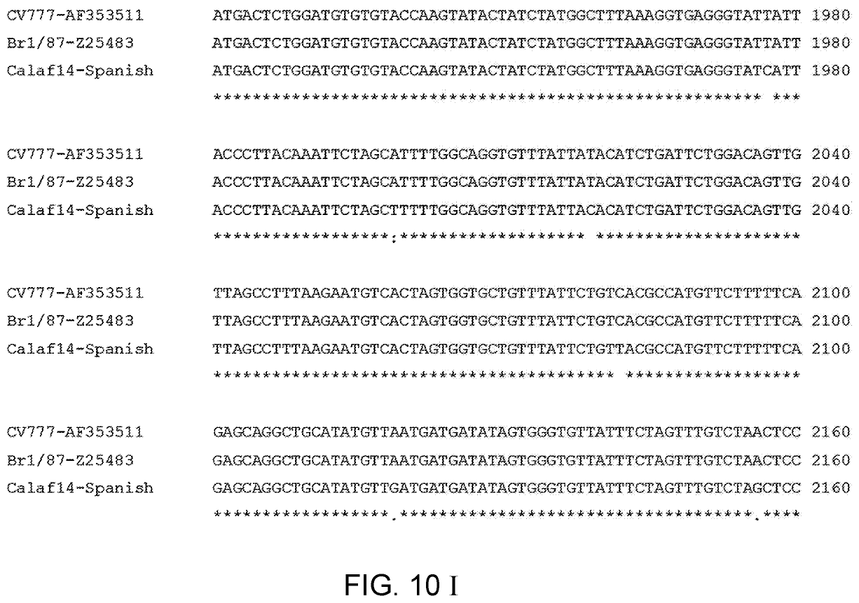

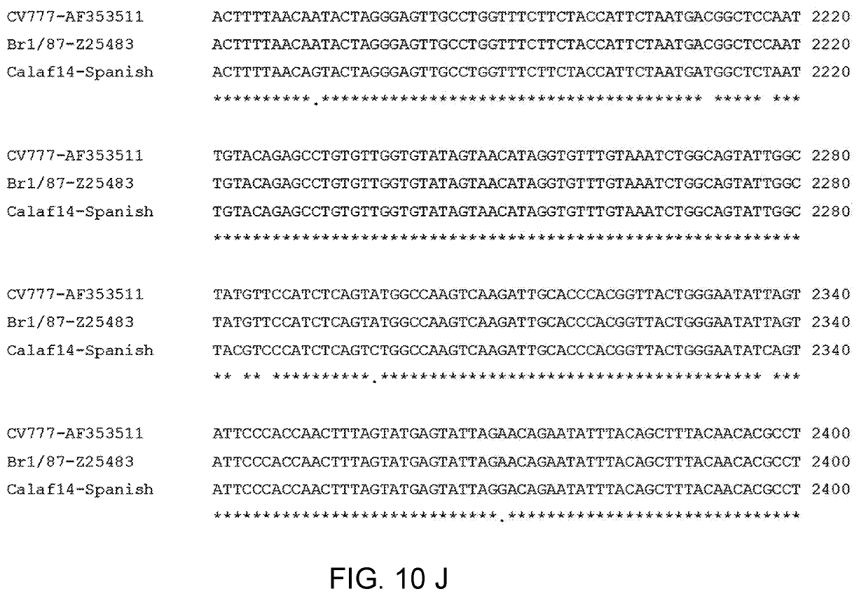

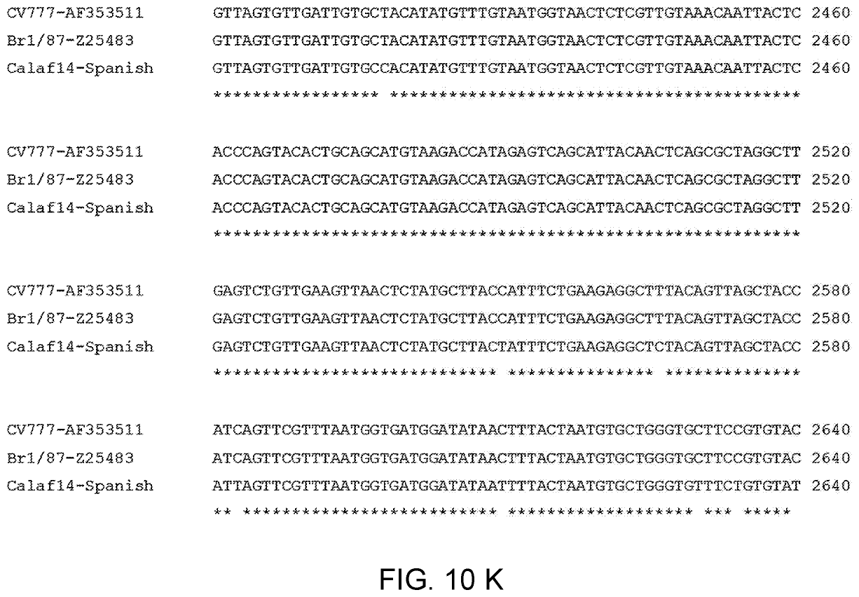

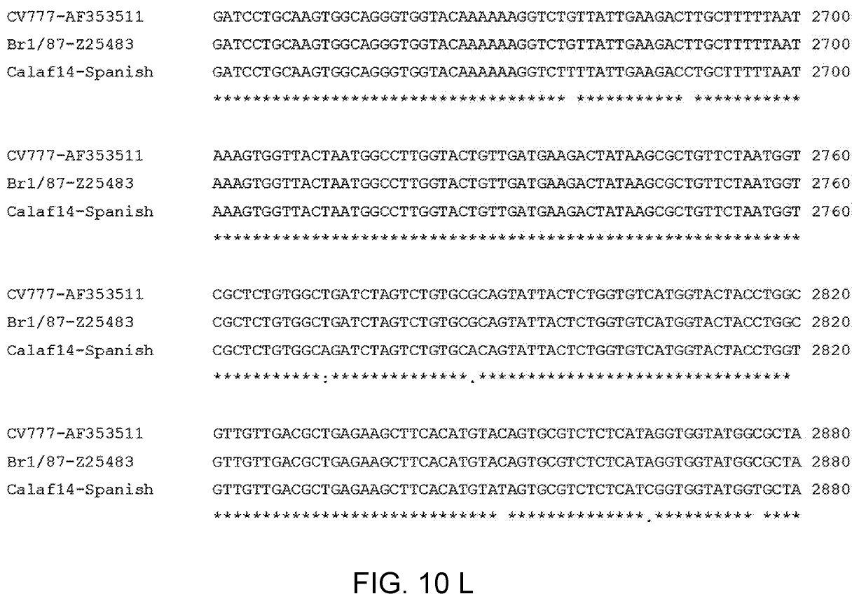

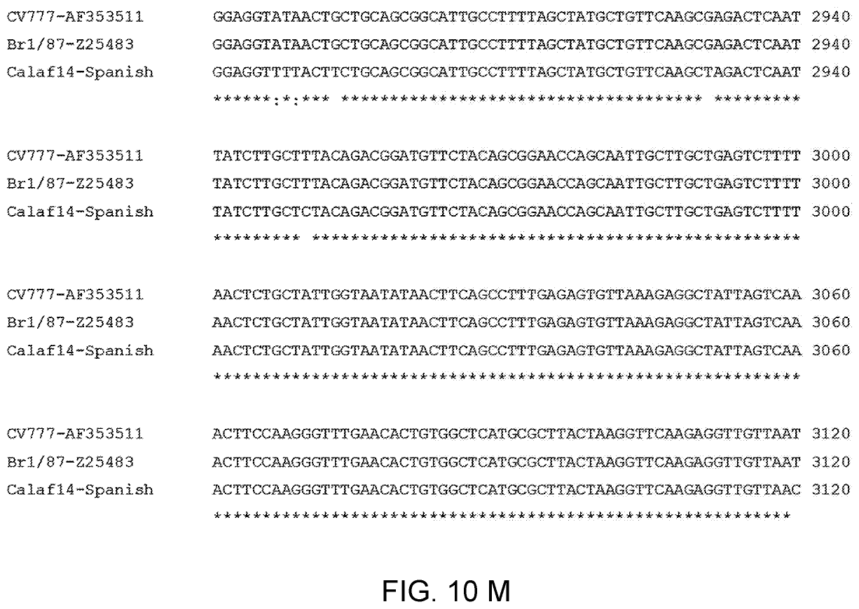

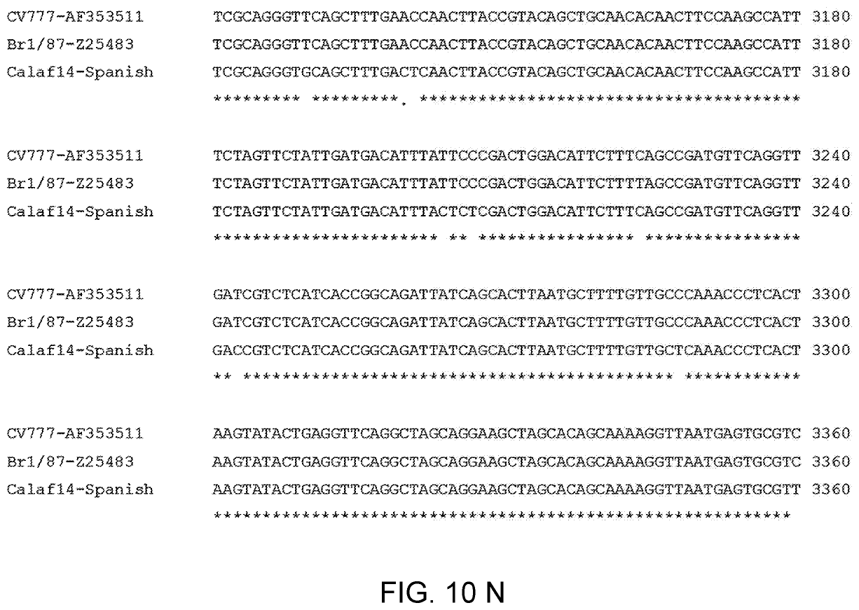

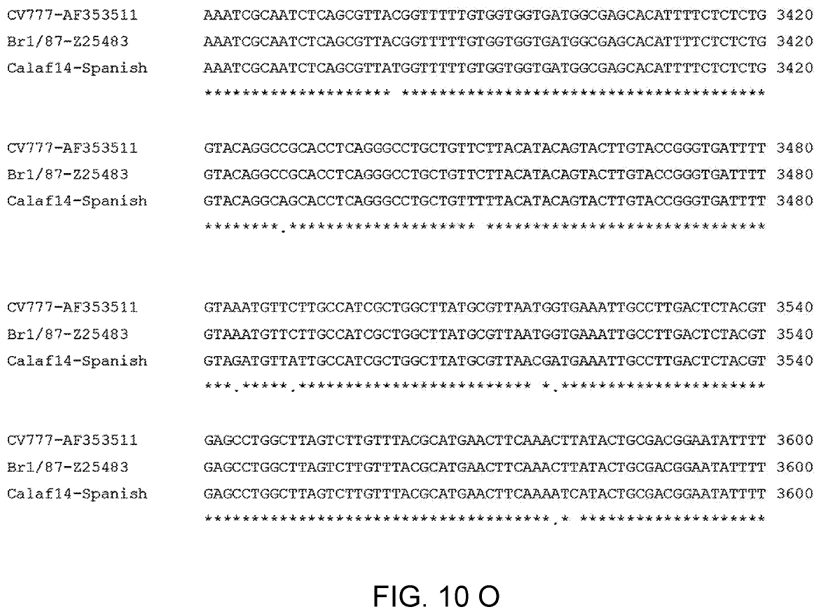

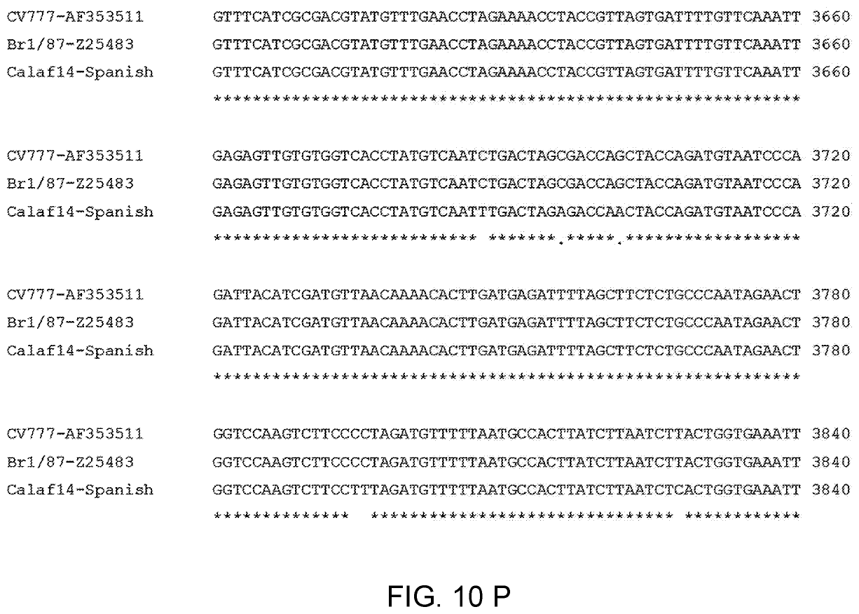

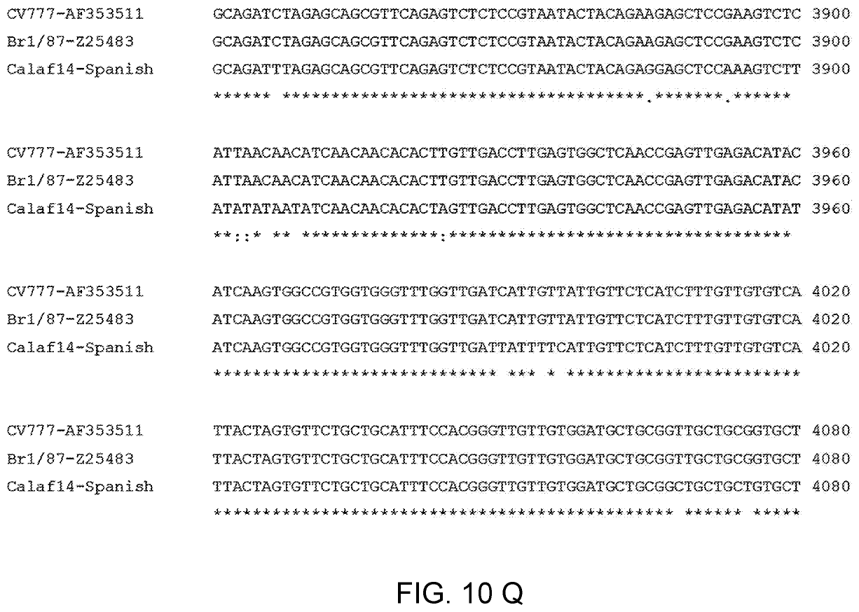

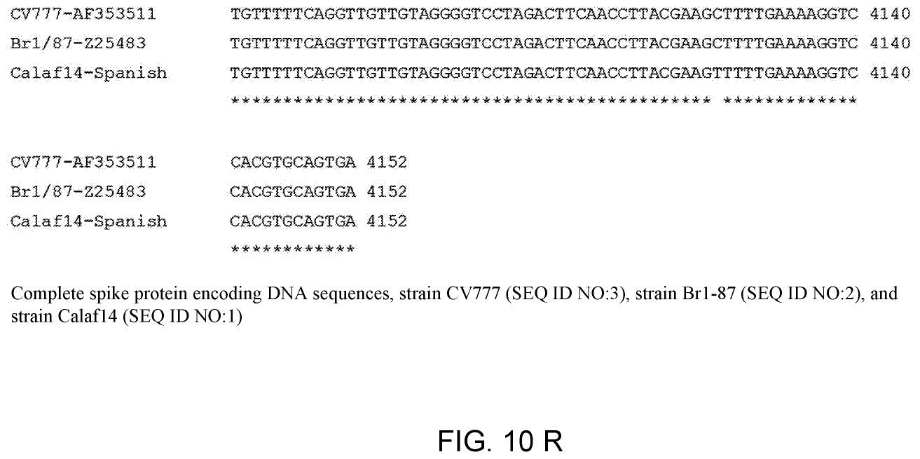

Complete S predicted protein sequences (1383 amino acids) were generated for the three isolates (SEQ ID NOS 4, 5 and 6) using Vector NTI Advance 11.5 software. Protein sequences were aligned using both Vector NTI Advance 11.5 and CLUSTAL 2.1 multiple sequence alignment. No insertions or deletions were detected when Calaf14 S protein (SEQ ID NO:4) was compared to CV777 (SEQ ID NO:6) and Br1/87 (SEQ ID NO:5) European isolates proteins. Nevertheless, analysis showed that identity between the two European reference isolates was of 99.71% whereas Calaf14 S protein showed a 95.81% of identity to Br1/87 and 96.1% to CV777 S protein (See FIG. 8).

It should be noted that Calaf14 is also an excellent strain from which to provide a vaccine (whether attenuated live or killed, in both cases either with or without adjuvant) that protects against PEDV challenge and disease, irrespective of whether the disease/challenge PEDV is: (1) of Asian origin including of INDEL types; (2) of European origin, when the European strain is a prototype strain such as was first detected in the 1970's or is any recently emerging strain, for example similar to North American INDELs; or (3) of North American origin, when the North American strain is a prototype strain, such as was first detected in 2013, or is reflective of emerging North American strains, such as INDELs; or (4) when the disease threat is posed by any combination of Asian, North American and European strains as disclosed herein.

The Calaf14 strain may be provided for use as a killed vaccine, following, for example, the preparatory methods described herein or other methods known in the art, to optionally include an adjuvant such as those adjuvant compositions described in the present specification. The Calaf14 strain may also be provided as an attenuated (i.e. modified) live vaccine, with or without an adjuvant, although those skilled in the art will recognize that only certain adjuvants are compatible with maintaining the viability of the live vaccine virus. Attenuation of the Calaf14 virus for a live vaccine so that it is insufficiently pathogenic to substantially harm the vaccinated target animal may be accomplished by known procedures, typically by serial passaging, as is recited in any of the following references which provide for attenuation of coronaviruses: B. Neuman et al., Journal of Virology, vol. 79, No. 15, pp. 9665-9676, 2005; J. Netland et al., Virology, v 399(1), pp. 120-128, 2010; Y-P Huang et al., “Sequence changes of infectious bronchitis virus isolates in the 3′ 7.3 kb of the genome after attenuating passage in embryonated eggs, Avian Pathology, v. 36 (1), (Abstract), 2007; and S. Hingley et al., Virology, v. 200(1) 1994, pp. 1-10. It has also been generally disclosed that INDEL-type strains are often less virulent toward swine (including sows and piglets) compared to prototype PEDV strains, thus permitting Calaf14 to be used as a live vaccine with little or no attenuation.

Generally speaking, it is also within the practice of the present invention to provide vaccines containing more than one PEDV isolate, whether the vaccine is a live or killed vaccine, and/or to vaccinate animals proximally in time with more than one vaccine composition to thus deliver more than one PEDV isolate as antigen. Representative combination vaccines (killed or live) of the invention include (a) use of Calaf14 with CV777 and/or Br1/87 European isolate, or other European isolate(s) whether prototype or emerging; (b) use of Calaf14 in combination with North American USA/Colorado/2013 GenBank No. KF272920, or any other North American prototype(s) and/or emerging North American (INDL) strain(s), (c) use of Calaf 14 with any Asian strain, and (d) use of Calaf14 with all combinations of the foregoing. Further all such multiple combinations may be further combined with a modified live (attenuated) or killed PDCoV virus.

### Example 7: Cross Protection Against European Strains, Additional Trial Results

The Porcine Epidemic Diarrhea Vaccine, Killed Virus, manufactured by Zoetis, is intended for pre-farrowing vaccination of sows and gilts against diarrheal disease in their neonatal pigs caused by PEDV. This killed vaccine was developed using a highly virulent American PEDV strain (USA/Colorado/2013) to be administered to intramuscularly to pregnant sows in two ml doses three weeks apart at 5 and 2 weeks pre-farrowing.

The objective of the study was to determine the immunogenicity of this vaccine, by infecting 4-6 day old piglets born from vaccinated pregnant sows with a new Spanish PEDV live isolate, Calaf14, as challenge. An efficacy study of the vaccine in pregnant sows was required to evaluate the maternal antibody protection against Porcine Epidemic Diarrhea virus, since PEDv induces gastro-intestinal disease, and protection against infection and disease against PEDv is mediated by maternally-derived antibodies. See Table 1 Ai B for design.

A total of 31 piglets born from sows vaccinated with the Inactivated PEDV vaccine (T02) and 21 from sows vaccinated with the placebo (T01) were included in the study. All piglets were challenged with the PEDV Spanish isolate at the age of 4 or 6 days. No mortality associated to PEDV challenge was detected in piglets from inactivated PEDV vaccine vaccinated sows (T02) whereas 23.8% challenge-associated mortality was reported for piglets from placebo vaccinated sows (T01).

After challenge, mild to severe digestive disorders including vomiting and aqueous yellow diarrhea were reported in 90.5% of piglets from placebo vaccinated sows; in piglets from PEDV killed virus vaccinated sows digestive disorders were observed in 48.4% of the piglets and ranged from mild to moderate. After challenge, 66.7% of piglets from placebo vaccinated sows experienced a mild to severe loss of general physical condition and/or dehydration whereas these signs were reported in only 3.2% of piglets from PEDV killed virus vaccinated sows and only mild dehydration was observed in these animals.

Body weight loss was detected ever after challenge in 42.9% of piglets from placebo vaccinated sows, ranging from mild to severe, whereas it was detected in 6.5% of animals from PEDV killed virus vaccinated sows as a mild degree.

Summary and frequency distribution of PEDV related clinical signs recorded after challenge with an heterologous PEDV strain (Spanish isolate, Calaf14) suggest that maternal antibody derived protection was obtained for piglets born from vaccinated sows with the PEDV inactivated vaccine. In conclusion, results suggest that the PEDV inactivated vaccine containing a US PEDV isolate as an antigen, is able to confer partial cross-protection to piglets born from vaccinated sows, in front of the challenge with an heterologous new PEDV Spanish isolate. Therefore, results suggest the suitability of the PEDV Vaccine, Killed Virus, manufactured by Zoetis, containing a US PEDV isolate as an antigen, to reduce the impact of an outbreak produced by new EU PEDV isolates.

TABLE 1A
Vaccination phase

| | | | | | Animals |
|---|---|---|---|---|---|
| | | | | | per |
| Treat- | Treatment | | Route | Day(s) | Treat- |
| ment | Description | Dosage | of Admin | of Admin | ment |
| T01 | Control | 2 ml | IM | 0 and 21 | 3 |
| | (Adjuvant | | | | |
| | Placebo) | | | | |
| T02 | Vaccine | 2 ml | IM | 0 and 21 | 5 |
| | (PEDV-1 | (Pre- | | | |
| | CO 2013 | inactivation | | | |
| | (PEDV-1 | titer of 7.5 | | | |
| | CO 2013 | TCID50/dose) | | | |
| | Killed Virus) | | | | |

At 5 weeks before the expected farrowing date, a dose of the CP was administered to T01 sows by IM route, and they were revaccinated 3 weeks later. Also, 5 weeks before the expected date farrowing a dose of the IVP was administered to T02 sows by IM route, and they were revaccinated 3 weeks later.

TABLE 1B
Challenge phase

| | | | | Day of | | Animals |
|---|---|---|---|---|---|---|
| Treatment | Treatment | | Route of | Challenge | End | per |
| group | Description | Dosage | Admin | (DC) | of Study | Treatment |
| T01 | PEDV Calaf14 | 10 ml of a 10⁻³ | Esophageal | 4-6 day-old | 3-4 days | All piglets |
| | Spanish | dilution of intestinal | gavage | | post- | from each |
| | isolate | homogenate stock | | | challenge | litter |
| | | (estimated 10²-10³ | | | | |
| | | PEDV genome | | | | |
| | | copies) | | | | |
| T02 | PEDV Calaf14 | 10 ml of a 10⁻³ | Esophageal | 4-6 day-old | 3-4 days | All piglets |
| | Spanish | dilution of intestinal | gavage | | post- | from each |
| | isolate | homogenate stock | | | challenge | litter |
| | | (estimated 10²-10³ | | | | |
| | | PEDV genome | | | | |
| | | copies) | | | | |

At 4 to 6 days of age all pigs from each litter were challenged with PEDv Calaf14 and 3 to 4 days post-challenge (end of the study), they were euthanized and necropsied.
Definition of Day 0: Day 0 was established as the day of first vaccination (5 weeks pre-farrowing). “IVP” means the experimental vaccine product, i.e. the Colorado 2013 killed material, as formulated above. CP means the control material (adjuvants plus diluent) without virus/viral antigen.
Randomization: Sows were grouped in two batches according to the expected farrowing date. Batch-1 included three sows and Batch-2 five. Sows from each batch were randomly allocated to experimental groups according to local internal procedures (function “random” of Microsoft Excel program: random number assigned to each animal, re-ordered in decreasing order, and sequential distribution to treatment group).
Vaccine: As aforementioned, the vaccine used is Zoetis PEDV vaccine, killed virus, “PEDV CO 2013 (NVSL)” adjuvanted with 5% Rehydragel and 5% Amphigen, and was formulated based on a pre-inactivation titer at 7.2 TCID₅₀/mL (i.e. 7.5 TCID₅/dose) for use as 2 ML intramuscular doses. Control vaccine material contained 5% Rehydragel and 5% Amphigen formulated with diluent rather than PEDv antigen. Vaccinations were conducted intramuscularly at Day 0 (right side of neck) and at Day 21 (left side of neck).
Further information concerning the challenge material: The challenge material was recovered from a clarified intestinal homogenate from a neonate piglet on a local Spanish farm, and was diluted just prior to inoculation to achieve an appropriate concentration, i.e. a targeted titer is 10⁷ to 10⁸ PEDV genome copies/10 mL dose, requiring an approximate 1000-fold dilution of intestinal homogenate, with the 10 ML dose being administered by esophageal gavage (virus named Calaf14).

### PEDV Disease-Related Mortality

When mortality was due to clinical signs associated to PEDV disease, it was summarized as challenge related mortality. Results are detailed below in Table 2.
From a total of 21 piglets from T01, 5 were euthanized due to PEDV related clinical signs, thus 23.8% challenge associated mortality was reported for T01 treatment group. No pigs died or were euthanized due to signs consistent with another disease.
No mortality associated to PEDV challenge was detected in T02 treatment group.

TABLE 2
PEDV challenge related mortality: number
and % of Animals for Each Treatment

| | PEDV challenge related? | | | | total |
|---|---|---|---|---|---|
| | NO | | YES | | observations |
| treatment number | number | % | number | % | number |
| T01 | 16 | 76.2 | 5 | 23.8 | 21 |
| T02 | 31 | 100.0 | 0 | 0.0 | 31 |
| total observations | 47 | 90.4 | 5 | 9.6 | 52 |

General physical condition and dehydration, digestive disorders, temperature, weight loss, depression and appetite loss were clinical signs associated to PEDV disease thus considered related to challenge, and are compiled in Table 3. Digestive disorders including vomiting and aqueous yellow diarrhea were reported in 90.5% of animals from treatment group T01 whereas it was observed in 48.4% of T02 group piglets. One case from T01 experienced severe digestive disorders reaching the end point criteria that justified its euthanasia for welfare reasons. After challenge, 66.7% of piglets from treatment group T01 experienced a loss of general physical condition and/or dehydration whereas it was reported in only 3.2% of piglets from T02. Reported dehydration for T01 piglets ranged from mild to severe (1 to 3 reported scores) and only mild dehydration was reported in one piglet from T02. None of the piglets from treatment group T02 experienced a loss of appetite ever after challenge whereas 14.3% (3 out of 21) of piglets from T01 did. Weight loss was defined as secondary efficacy variable. Depression was observed after challenge in 66.7% of piglets from treatment group T01. Depressive status ranged from mild to moderate. Depression was also observed in 9.7% of piglets from T02. Abnormal temperature values (T^{a}>40.5° C. or T^{a}<37.0° C.) were recorded ever after challenge in 9.5% of piglets from T01. None of the piglets from treatment group T02 had abnormal temperature values.

TABLE 3
clinical sign ever present: frequency distributions by treatment

| | | Clinical Observations (Percentage of Animals, %) | | | | | | | | |
|---|---|---|---|---|---|---|---|---|---|---|
| | | General physical | | | | | | | | |
| | Ever | condition and | | Weight | | | | Traumatisms and | | |
| Treatment | present | dehydration | Temperature | loss | Depression | Appetite | Digestive | locomotors disorders | Respiratory | Other |
| T01 | No | 33.3 | 90.5 | 57.1 | 33.3 | 85.7 | 9.5 | 85.7 | 95.2 | 76.2 |
| | Yes | 66.7 | 9.5 | 42.9 | 66.7 | 14.3 | 90.5 | 14.3 | 4.8 | 23.8 |
| T02 | No | 96.8 | 100.0 | 93.5 | 90.3 | 100.0 | 51.6 | 96.8 | 100.0 | 96.8 |
| | Yes | 3.2 | 0.0 | 6.5 | 9.7 | 0.0 | 48.4 | 3.2 | 0.0 | 3.2 |

In summary, the clinical data results from this study indicate that the PEDV inactivated vaccine containing a US PEDV isolate as an antigen, is able to confer at least partial cross-protection to piglets born from vaccinated sows, in front of the challenge with an heterologous new PEDV Spanish isolate, Calf14. Therefore, results suggest the suitability of the PEDV Vaccine, Killed Virus, manufactured by Zoetis, containing a US PEDV isolate as an antigen, to reduce the impact of an outbreak produced by a new EU PEDV isolate

### Example 8: Isolation, Propagation, and Inoculation of CDCD Pigs with PDCoV USA/Indiana/2014/8501010 and NVSL PDCoV USA/Michigan/8977/2014

Approximately 1 cm³ of tissue was used for extraction of PDCoV virus. The tissue was chopped into fine pieces using a sterile scalpel and scissors in a sterile Petri dish. Work was done in a Bio-safety cabinet to ensure aseptic conditions. Two ml of sterile PBS was added to the Petri dish to collect tissue and material was transferred to a 15 ml conical tube. Tissue was homogenized with a Qiagen TissueRuptor at 80% of maximum by pulsing for a total of 30 seconds. Homogenization was performed in an ice bucket to lessen the effect of heat on the PDCoV virus. The homogenized material was filtered through a 0.45 μM filter and 60 μl of material was used for RNA isolation and PDCoV qPCR to confirm the presence of the PDCoV virus. The filtered material containing PDCoV virus was further diluted 1:2 in sterile PBS, and then filtered through a 0.20 μM filter.

The sterile-filtered PDCoV homogenate was used to infect confluent monolayers of Swine Testicle (ST) cells by transferring 1 ml of filtered material to a T-25 flask containing 2.8×10⁶ cells, planted 4 days prior to infection. The T-25 flasks of confluent ST cells were washed 2× with sterile PBS, and 1× with PMEM media containing 20 g/ml geneticin and 1 μg/ml TPCK trypsin (equivalent to 4.9 USP units/ml). A total of three T-25 flasks with a confluent monolayer of ST cells were infected for 1 hour at 37° C. in a 5% CO₂ incubator, with gentle swirling every 15 minutes to ensure the virus was evenly distributed to all cells. Five mls of PMEM media containing 20 μg/ml geneticin, 2 mM L-glutamine, and either 1 μg/ml TPCK typsin (equivalent to 4.9 USP units/ml), 3 μg/ml TPCK trypsin (equivalent to 14.6 USP units/ml), or 5 μg/ml TPCK trypsin (equivalent to 24.5 USP units/ml) was added to virus-treated flasks. Flasks were allowed to incubate for 3 days, with sampling occurring each day. After 3 days, flasks were frozen at −80° C., then thawed at 37° C., and the flask contents were placed in a 15 ml conical tube and centrifuged to remove cellular debris. The supernatant was collected, and this virus-containing material is considered as Passage 1 of the virus, PDCoV USA/Indiana/2014/8501010. One ml of the total volume from the all 3 flasks was then used for Passage 2 of the virus onto three separate T-25 flasks of confluent ST cells. One ml of Passage 1 PDCoV material was used to infect a T-25 flask containing 2.8×10⁶ cells seeded 3 to 4 days prior. Cells were first washed 2× with sterile PBS, and 1× with PMEM media containing 20 g/ml geneticin and 1 μg/ml TPCK trypsin (equivalent to 4.9 USP units/ml). Cells were infected for 1 hour at 37° C. in a 5% CO₂ incubator, with gentle swirling every 15 minutes to ensure virus was evenly distributed to cells. Five mls of PMEM media containing 20 g/ml geneticin, 2 mM L-glutamine, and either 1 μg/ml TPCK typsin (equivalent to 4.9 USP units/ml), 3 g/ml TPCK trypsin (equivalent to 14.6 USP units/ml), or 5 g/ml TPCK trypsin (equivalent to 24.5 USP units/ml), corresponding to the initial trypsin concentration at infection that was added to virus-treated flasks. This procedure was repeated out to Passage 15, with the 3 g trypsin infection media sample and 12 mls of PDCoV USA/Indiana/2014/8501010 at each passage being retained.

Passage 1 material that was sampled daily was used in a PDCoV M gene-based RT-qPCR assay to monitor growth of the virus with the following primers: Forward Primer: 5′-ATCGACCACATGGCTCCAA-3′ (SEQ ID NO:8); Reverse Primer: 5′-CAGCTCTTGCCCATGTAGCTT-3′ (SEQ ID NO:9); and Probe: 5′/56FAM/-CACACCAGTCGTTAAGCATGGCAAGCT/3BHQ_1/3′ (see SEQ ID NO:10). Briefly, 140 μl of each time-point sample virus was used for RNA isolation. Five microliters of extracted RNA was then subjected to RT-qPCR to determine final cycle threshold (Ct) value and copy number of each sample. At day 0, all three infected flasks had a Ct value of between 22 and 23, which corresponds to between 2.34×10⁵ and 3.24×10⁵ copies per sample. Each day sampled thereafter results in a decrease in Ct value, which correlates to an increase in viral copy number for each sample, indicating replication and growth of the virus. Summarized in Table 4 are the Ct value and corresponding copy number data for the virus.

TABLE 4
Growth Monitoring of PDCoV USA/Indiana/2014/8501010

| Cycle Threshold (Ct) Value | | | | Copy Number/5 ul Value | | | |
|---|---|---|---|---|---|---|---|
| | 1 μg | 3 μg | 5 μg | | 1 μg | 3 μg | 5 μg |
| | tryp- | tryp- | tryp- | | tryp- | tryp- | tryp- |
| Day | sin | sin | sin | Day | sin | sin | sin |
| 0 | 23.13 | 22.67 | 22.81 | 0 | 2.32E+05 | 3.47E+05 | 3.24E+05 |
| 1 | 18.34 | 17.97 | 17.50 | 1 | 6.88E+06 | 1.01E+07 | 1.13E+07 |
| 2 | 18.73 | 18.05 | 17.55 | 2 | 4.83E+06 | 8.64E+06 | 1.22E+07 |
| 3 | 18.37 | 17.85 | 17.55 | 3 | 5.38E+06 | 1.19E+07 | 6.3E+07 |

Plastic flasks or roller bottles were used for growing and expanding ST cell cultures. Plastic flasks, roller bottles, and bioreactors were used for PDCoV virus propagation. Cells were washed to remove serum prior to inoculation with virus. The virus was diluted in PMEM media containing 20 g/ml geneticin, 2 mM L-glutamine, and 1 μg/ml TPCK typsin (equivalent to 4.9 USP units/ml), and added directly to the cell monolayer. When bioreactors were used for virus propagation, trypsinized cells were transferred from the roller bottles, and a final cell passage grown in uninoculated cell growth medium was used to seed the bioreactor. Microcarriers for the bioreactors were prepared and added to the ST cells in the bioreactor. The seed virus was diluted to an appropriate volume within a multiplicity of infection (MOI) range of 0.0001 to 10.0. Growth of virus was monitored by visualizing CPE of virus infected cells and by RT-qPCR. The NVSL virus strain, PDCoV USA/Michigan/8977/2014 (see SEQ ID NO:12 for corresponding encoding DNA), was passaged to Passage 22.

The PDCoV virus causes observable cytopathic effect (CPE). Virus was harvested when viral-induced CPE reached 50-100% and infected cells began sloughing off into the medium (cell monolayer loss exceeding 50%). The roller bottle vessels were removed from the incubator, and inspected microscopically for both CPE and evidence of microbial contamination. Following the examination, the antigen fluid was harvested into appropriate sterile containers in an aseptic manner. Bioreactor fluids were examined microscopically for evidence of microbial contamination, and for the presence of desired cytopathic effects (CPE).

Following examination, the viral fluids were passed through a:100 micron filter or stainless steel mesh screen to remove microcarriers, and harvested into appropriate sterile containers in an aseptic manner. Fluids were stored at 2° C.-7° C. for a maximum of 24 hours until inactivation.

In separate tests, (1) original intestinal homogenate (source of PDCoV USA/Indiana/2014/8501010); (2) Passage 4 of strain PDCoV USA/Indiana/2014/8501010 (see SEQ ID NO:11 for corresponding encoding DNA), and (3) Passage 10 of strain PDCoV USA/Michigan/8977/2014 (see SEQ ID NO:12 for corresponding encoding DNA), were injected into 3 day old CDCD (Caesarian-derived, colostrum deprived) pigs to expand the virus material, and PDCoV virulence in pigs was assessed by monitoring clinical signs (diarrhea and vomiting), histopathology, and RT-qPCR of fecal material. Pigs were placed in assigned pens in a BSL-2 facility, with each treatment group being housed in a separate room to avoid cross-contamination. The peak clinical signs and fecal shedding appeared between 16-24 hours for the PDCoV USA/Indiana/2014/8501010 strain (see SEQ ID NO:11), and at 3 days post-inoculation for the PDCoV USA/Michigan/8977/2014 strain (see SEQ ID NO:12).

In addition to being a useful killed vaccine, it should be noted that passage 10 of PDCoV USA/Michigan/8977/2014 is sufficiently attenuated as to define the approximate minimum threshold of a passaged isolate that could be recommended for a live vaccine, although a higher number of passages would be preferred.

### Example 9: Preparation and Testing of a Vaccine Based on Porcine Deltacoronavirus Isolate PDCoV USA/Michigan/8977/2014

Harvested PDCoV antigen was concentrated 20×prior to inactivation with a 5 mM binary ethylenimine (BEI) solution. The mixture is cyclized for 60-80 minutes at 36±2° C. Following the addition of inactivant, the antigen was thoroughly mixed and transferred to an inactivation vessel for the duration of the process (≥48 hours, with agitation). Neutralization of the inactivated antigen fluids was facilitated through the addition of sterile 1 M Sodium Thiosulfate, to a final concentration of approximately 20-25 mM. Post-inactivated/neutralized antigen production fluids were tested for sterility and completeness of inactivation, and stored at 2-7° C. for future use in vaccine serial formulation.

A vaccine containing the following components was formulated: 7.42 log₁₀TCID₅₀ of PDCoV USA/Michigan/8977/2014 (see SEQ ID NO:12) virus per 2 ml dose; 5% (v/v) Rehydragel® LV; 25% (v/v) of “20% Amphigen” (i.e. it is further 4-fold diluted); and 0.01% (w/v) of merthiolate.

Killed PDCoV USA/Michigan/8977/2014 virus was also adjuvanted with TXO, and used for vaccination. TXO provided the following components per 1 ml dose of vaccine: 50 ug “CpG 23877” (see SEQ ID NO: 8 as listed in the WO2015/042369 publication), 10 mg DEAE-Dextran, DRAKEOL 6VR (45% w/v), Span-80 (6.3% v/v), Tween-80 (1.45% v/v) and 10 mM PBS.

As is understood in the art, the order of addition of components can be varied to provide the equivalent final vaccine composition. For example, an appropriate dilution of killed virus in buffer can be prepared. An appropriate amount of Rehydragel® LV (about 2% aluminum hydroxide content) stock solution can then be added, with blending, in order to permit the desired 5% (v/v) concentration of Rehydragel® LV in the actual final product. Once prepared, this intermediate stock material is combined with an appropriate amount of “20% Amphigen” stock (as generally described above, and already containing necessary amounts of Tween 80 and Span 80) to again achieve a final product having 25% (v/v) of “20% Amphigen”. An appropriate amount of 10% merthiolate can finally be added.

The vaccinate compositions of the invention permit variation in all of the ingredients, such that the total dose of antigen may be varied preferably by a factor of 100 (up or down) compared to the antigen dose stated above, and most preferably by a factor of 10 or less (up or down). Similarly, surfactant concentrations (whether Tween or Span) may be varied by up to a factor of 10, independently of each other, or they may be deleted entirely, with replacement by appropriate concentrations of similar materials, as is well understood in the art.

Porcine serum generated from the pigs vaccinated with inactivated PDCoV adjuvanted with Amphigen®/Rehydragel® LV or TXO were tested in a serum neutralization (SN) assay as follows: Porcine serum from each treatment group was pooled and heat inactivated at 56° C. for 30 minutes. Serum samples were diluted 2-fold by mixing 500 μl of the serum with 500 μl PMEM media supplemented with 20 g/ml geneticin, 2 mM L-glutamine and 1 μg/ml TPCK typsin (equivalent to 4.9 USP units/ml). PDCoV live virus at dilutions ranging from log₁₀TCID₅₀=5.0 to log₁₀TCID₅₀=2.0 were added to the diluted serum and incubated for 1 hour at room temperature. The serum/virus mixture was inoculated onto 96-well plates seeded with confluent ST cells, and incubated for 4 days at 37° C. and 5% CO₂ The plates were then fixed with 80% acetone in a water mixture for 15 minutes. The mixture was then removed, and plates were air-dried for 15 minutes to remove the remaining acetone. Plates were stained with rabbit anti-PDCoV S1 serum primary antibody, and goat anti-rabbit Alexa Fluor® 488-labelled secondary antibody (Jackson ImmunoResearch), prior to reading plates on a fluorescent microscope. The serum neutralization titer was calculated by determining the lowest dilution of serum where PDCoV growth was 100% inhibited, and applying the Spearman-Karber method to calculate titer values.

It was determined that the serum from pigs vaccinated with inactivated PDCoV adjuvanted with either Amphigen®/Rehydragel® LV, or TXO, successfully neutralized the growth of PDCoV virus on ST cells at all virus inoculum concentrations tested. In general, the group vaccinated with inactivated PDCoV/TXO adjuvant gave higher SN titers (see Table 5) than the Amphigen®/Rehydragel® LV-adjuvanted group.

TABLE 5
Serum Neutralising (SN) Titers of Inactivated PDCoV Vaccinated Pigs

| | PDCoV Virus Titer | | | |
|---|---|---|---|---|
| | Log₁₀TCID₅₀ = | Log₁₀TCID₅₀ = | Log₁₀TCID₅₀ = | Log₁₀TCID₅₀ = |
| Vaccine Treatment | 5 . . . 0 | 4 . . . 0 | 3 . . . 0 | 2 . . . 0 |
| Saline | <2 | <2 | <2 | <2 |
| PDCoV + Amphigen/ | 128 | 128 | 256 | 384 |
| Rehydragel | | | | |
| PDCoV + TXO | 256 | 384 | 512 | 1024 |

### Example 10: Cloning, Expression, and Inoculation of Pigs with S1 Protein; Expression of N Protein

The complete genome sequence of Porcine Deltacoronavirus isolate USA/IA/2014/8734 has been published and deposited in GenBank under the accession number KJ567050. From that sequence, a synthetic S1 gene with a 3′ His-tag was generated, and cloned into a proprietary mammalian expression vector. The S1 protein was expressed in Human Embryonic Kidney (HEK) cells, and purified by immobilized metal affinity chromatograpy (IMAC). A 40 μg dose of purified S1 protein was adjuvanted either with 5% (v/v) Rehydragel® LV and 25% (v/v) of “20% Amphigen”, or with TXO adjuvant, and injected into pigs to generate a humoral immune response through the production of antibodies to the S1 protein.

Porcine serum generated from the pigs vaccinated with PDCoV S1 protein adjuvanted with Amphigen®/Rehydragel® LV or with TXO were tested in a serum neutralization (SN) assay as follows:

Porcine serum from each treatment group was pooled, and heat inactivated at 56° C. for 30 minutes. Serum samples were diluted 2-fold by mixing 500 μl of the serum with 500 μl PMEM media, supplemented with 20 g/ml geneticin, 2 mM L-glutamine, and 1 g/ml TPCK typsin (equivalent to 14.6 USP units/ml). PDCoV virus at dilutions ranging from log₁₀TCID₅₀=5.0 to log₁₀TCID₅₀=2.0 were added to the diluted serum, and incubated for 1 hour at room temperature. The serum/virus mixture was inoculated onto 96-well plates seeded with confluent ST cells, and incubated for 4 days at 37° C. and 5% CO₂ The plates were then fixed with 80% acetone in water mixture for 15 minutes, after which the mixture was removed, and plates were air-dried for 15 minutes to remove the remaining acetone. Plates were then stained with rabbit anti-PDCoV S1 serum primary antibody, and goat anti-rabbit Alexa Fluor-labelled secondary antibody, prior to reading plates on a fluorescent microscope. The serum neutralization titer was calculated by determining the lowest dilution of serum where PDCoV growth was inhibited and applying the Spearman-Karber method to calculate titer values.

It was determined that the serum from pigs vaccinated with PDCoV S1 protein advuanted with either Amphigen®/Rehydragel® LV or TXO successfully neutralized the growth of PDCoV virus on ST cells at all virus inoculum concentrations tested. In general, the group vaccinated with PDCoV S1 protein adjuvanted with TXO gave higher SN titers (see Table 6) than the Amphigen®/Rehydragel® LV-adjuvanted group.

TABLE 6
Serum Neutralising (SN) Titers of PDCoV S1 Vaccinated Pigs

| | PDCoV Virus Titer | | | |
|---|---|---|---|---|
| | Log₁₀TCID₅₀ = | Log₁₀TCID₅₀ = | Log₁₀TCID₅₀ = | Log₁₀TCID₅₀ = |
| Vaccine Treatment | 5.0 | 4.0 | 3.0 | 2.0 |
| Saline | <2 | <2 | <2 | <2 |
| PDCoV S1 + Amphigen/ | 24 | 32 | 64 | 192 |
| Rehydragel | | | | |
| PDCoV S1 + TXO | 128 | 192 | 384 | 536 |

The nucleocapsid (N) nucleotide sequence from PDCoV isolate USA/IA/2014/8734 was used to make a synthetic gene for cloning and expression of the N protein in both a pET100 vector, and a proprietary heat-inducible bacterial expression vector. The pET100 vector contains a 6×His tag for detection and purification of the expressed protein. Both constructs were transformed into E. coli, and expressed by induction with either 1 mM IPTG (pET100) or heat (heat-inducible vector). The bacterial expression resulted in an ˜51 kDa protein being expressed. This resulting protein will be purified and used as a reagent for antibody generation.

### Example 11: Efficacy of Monovalent PDCoV Vaccine and Bivalent (PDCoV+PEDV)Vaccine

In order to assess the efficacy in pregnant sows of a monovalent inactivated PDCoV vaccine, as well as a bivalent inactivated PDCoV/PEDV vaccine, the following experiments are carried out. PDCoV strain USA/Michigan/8977/2014 (see SEQ ID NO:12) is cultured, and vaccines prepared as described previously. A bivalent vaccine containing PEDV strain USA/Colorado/2013 (see SEQ ID NO:7) and PDCoV strain USA/Michigan/8977/2014 is also prepared. The vaccines are given intramuscularly to pregnant sows as two doses, 2 ML each, three weeks apart, at five and two weeks pre-farrowing.

Pregnant sows are included in the study. At 5 weeks before farrowing, a dose of each inactivated vaccine is administered to sows by the IM route; 1 or more sows remain unvaccinated (controls). Three weeks later, vaccinated sows receive a second dose. After farrowing, approximately at 0-5 days of age, all piglets are challenged with either the Spanish PEDV strain Calaf14 (see SEQ ID NO: 1 for S-protein encoding sequence), or the PDCoV strain USA/Indiana/2014/8501010 (see SEQ ID NO:11). Twice daily after challenge, all piglets are evaluated for the presence of clinical signs (including diarrhea); rectal temperatures are taken; body weights are measured; and fecal swabs are taken, to perform either a PEDV-specific or PDCoV-specific RT-qPCR assay. At day 3 to 7 after challenge, all piglets are euthanized and necropsied; gut tissue samples are also removed.

1-24. (canceled)

25. A vaccine composition comprising inactivated porcine epidemic diarrhea virus (PEDV), adjuvanted as an oil-in-water emulsion, wherein the adjuvant components include Amphigen® and aluminum hydroxide.

26. The vaccine composition of claim 25 wherein the inactivated PEDV is strain USA/Colorado/2013, whose genomic sequence is deposited in GenBank as accession No. KF272920.

27. The vaccine composition of claim 25 wherein the final concentration of 20% Amphigen is 25% (v/v).

28. The vaccine composition of claim 25 further comprising inactivated porcine deltacoronavirus (PDCoV).

29. The vaccine composition of claim 28 wherein the PDCoV is selected from the group consisting of USA/Michigan/8977/2014, whose sequence is deposited as GenBank accession No. KM012168, and strain USA/Indiana/2014/8501010.

30. The vaccine composition of claim 28 wherein the final concentration of 20% Amphigen is 25% (v/v).


SEQUENCE LISTING (12 sequences)

SEQ ID NO: 1 - DNA, 4152 bp, Artificial Sequence
  DNA sequence encoding spike protein of PEDV strain Calaf14
atgaagtctttaaattacttctggttgttcttaccagtactttcaacactcagcctaccacaagatgtcactaggtgccagtccactattaacttcaggcggttcttttcaaaatttaatgtgcaggcacctgctgtcgttgtgttgggtggttatctacctagtatgaactcctctagctggtactgtggcacaggtcttgaaactgctagtggcgtgcatggtattttcctcagttacatcgatgctggtcagggctttgagattggcatttcacaggagccgtttgatcctagtggttaccagctttatttacataaggccactaatggtaaccataatgctattgcacgactgcgcatttgccagtttccaaataataaaacattgggccctactgttaatgatgttacaacaggtcgtaactgcctattcaacaaagccattccagcttatatgcaggatggaaaaaacatcgttgtcggcataacatgggacaatgatcgtgtcactgtttttgctgacaagatctatcatttttatctcaaaaatgattggtcccgtgttgcgacaagatgttacaataaaagaagttgtgctatgcaatatgtttatacacctacctactacatgcttaatgttactagtgcaggtgaggatggcatttattatgaaccatgtacagctaattgcagtggttacgctgccaatgtgtttgccactgattctaatggccacataccagaaggttttagttttaataattggtttcttttgtccaatgattccactttgttgcatggtaaggtggtttccaaccaacctttgttggtcaattgtcttttggccattcctaagatttatggactaggccaatttttctcattcaatcaaacgatggatggcgtttgtaatggagctgctgcgcagcgtgcaccagaggctctgaggtttaatattaatgacacctctgtcattcttgctgaaggctcaattgtacttcacactgctttaggaacaaatctttcttttgtttgcagtaattcttcagatcctcatttagctaccttcaccatacctctgggtgctacccaagtaccctattattgttttcttaaagtggatacttacaactccactgtttataaatttttggctgttttacctcctaccgtcagggaaattgtcatcaccaagtatggtgatgtttatgtcaatgggtttggatacttgcatctcggtttgttggatgctgtcacaattaatttcactggtcatggcactgacgatgatgtttctggtttttggaccatagcatcgactaattttgttgatgcactcatcgaagttcaaggaactgccattcagcgtattctttattgtgatgatcctgttagccaactcaagtgttctcaggttgcttttgaccttgacgatggtttttaccctatttcttctagaaaccttctgagtcatgaacagccaatttcttttgttactctgccatcatttaatgatcattcttttgttaacattactgtctctgcttcctttggtggtcatagtggtgccaaccttattgcatctgacactactatcaatgggtttagttctttctgtgttgacactagacaatttaccatttcactgttttataacgttacaaacagttatggttatgtgtctaaatcacaggacagtaattgccctttcaccttgcaatctgttaatgattacctgtcttttagcaaattttgtgtttccaccaaccttttggctagtgactgtaccatagatctttttggttaccctgagtttggtagtggtgttaagtttacgtccctttactttcaattcacaaagggtgagttgattactggcacgcctaaaccacttgaaggtgtcacggacgtttcttttatgactctggatgtgtgtaccaagtatactatctatggctttaaaggtgagggtatcattacccttacaaattctagctttttggcaggtgtttattacacatctgattctggacagttgttagcctttaagaatgtcactagtggtgctgtttattctgttacgccatgttctttttcagagcaggctgcatatgttgatgatgatatagtgggtgttatttctagtttgtctagctccacttttaacagtactagggagttgcctggtttcttctaccattctaatgatggctctaattgtacagagcctgtgttggtgtatagtaacataggtgtttgtaaatctggcagtattggctacgtcccatctcagtctggccaagtcaagattgcacccacggttactgggaatatcagtattcccaccaactttagtatgagtattaggacagaatatttacagctttacaacacgcctgttagtgttgattgtgccacatatgtttgtaatggtaactctcgttgtaaacaattactcacccagtacactgcagcatgtaagaccatagagtcagcattacaactcagcgctaggcttgagtctgttgaagttaactctatgcttactatttctgaagaggctctacagttagctaccattagttcgtttaatggtgatggatataattttactaatgtgctgggtgtttctgtgtatgatcctgcaagtggcagggtggtacaaaaaaggtcttttattgaagacctgctttttaataaagtggttactaatggccttggtactgttgatgaagactataagcgctgttctaatggtcgctctgtggcagatctagtctgtgcacagtattactctggtgtcatggtactacctggtgttgttgacgctgagaagcttcacatgtatagtgcgtctctcatcggtggtatggtgctaggaggttttacttctgcagcggcattgccttttagctatgctgttcaagctagactcaattatcttgctctacagacggatgttctacagcggaaccagcaattgcttgctgagtcttttaactctgctattggtaatataacttcagcctttgagagtgttaaagaggctattagtcaaacttccaagggtttgaacactgtggctcatgcgcttactaaggttcaagaggttgttaactcgcagggtgcagctttgactcaacttaccgtacagctgcaacacaacttccaagccatttctagttctattgatgacatttactctcgactggacattctttcagccgatgttcaggttgaccgtctcatcaccggcagattatcagcacttaatgcttttgttgctcaaaccctcactaagtatactgaggttcaggctagcaggaagctagcacagcaaaaggttaatgagtgcgttaaatcgcaatctcagcgttatggtttttgtggtggtgatggcgagcacattttctctctggtacaggcagcacctcagggcctgctgtttttacatacagtacttgtaccgggtgattttgtagatgttattgccatcgctggcttatgcgttaacgatgaaattgccttgactctacgtgagcctggcttagtcttgtttacgcatgaacttcaaaatcatactgcgacggaatattttgtttcatcgcgacgtatgtttgaacctagaaaacctaccgttagtgattttgttcaaattgagagttgtgtggtcacctatgtcaatttgactagagaccaactaccagatgtaatcccagattacatcgatgttaacaaaacacttgatgagattttagcttctctgcccaatagaactggtccaagtcttcctttagatgtttttaatgccacttatcttaatctcactggtgaaattgcagatttagagcagcgttcagagtctctccgtaatactacagaggagctccaaagtcttatatataatatcaacaacacactagttgaccttgagtggctcaaccgagttgagacatatatcaagtggccgtggtgggtttggttgattattttcattgttctcatctttgttgtgtcattactagtgttctgctgcatttccacgggttgttgtggatgctgcggctgctgctgtgcttgtttttcaggttgttgtaggggtcctagacttcaaccttacgaagtttttgaaaaggtccacgtgcagtga

SEQ ID NO: 2 - DNA, 4152 bp, Artificial Sequence
  DNA sequence encoding spike protein of PEDV strain Br1-87
atgaggtctttaatttacttctggttgctcttaccagtacttccaacactcagcctaccacaagatgtcactaggtgccagtctactactaactttaggcggttcttttcaaaatttaatgttcaggcacctgccgtcgtcgttttgggtggttacctacctagtatgaactcttctagctggtactgtggcacaggcattgaaactgctagtggcgttcatggtatttttctcagctacatcgattctggtcagggctttgagattggcatttcgcaagagccgtttgatcctagtggttaccagctttatttacataaggccactaatggtaacactaatgctactgcacgactgcgcatttgccagtttcccgataataaaacattgggccctactgttaatgatgttacaacaggtcgtaactgcctattcaacaaagccattccagcttatatgcgtgatggaaaagatattgttgtcggcataacatgggataatgatcgtgtcactgtttttgctgacaagatctatcatttttatcttaaaaatgattggtcccgcgttgcgacaagatgttacaatcgcagaagttgtgctatgcaatatgtttatacacctacctactacatgcttaatgttactagtgcaggtgaggatggcatttattatgaaccctgtacagctaattgcactggttacgctgccaatgtatttgccactgattccaatggccatataccagaaggttttagttttaataattggtttcttttatccaatgactccactttgttgcatggtaaagtggtttccaaccaacccttgttggtcaattgtcttttggccattcctaagatttatggactaggccaatttttctcattcaatcacacgatggatggcgtttgtaatggagctgctgtggatcgtgccccagaggctctgaggtttaatattaatgacacctccgtcattcttgctgaaggctcaattgtacttcatactgctttaggaacaaatctttcttttgtttgcagtaattcctcagatcctcatttagccatctttgccatacctctgggtgctactgaagtaccctactattgctttcttaaagtggatacttacaactccactgtttataaattcttggctgttttaccttctactgtcagggaaattgtcatcaccaagtatggtgatgtttatgtcaatgggtttggctatttgcatctcggtttgttggatgctgtcacaatttatttcactggtcatggcactgacgatgacgtttcaggtttctggaccatagcatcgactaattttgttgatgcactcatcgaggttcaaggaacttccattcagcgtattctttattgtgatgatcctgttagccaactcaagtgttctcaggttgcttttgaccttgacgatggtttttaccccatctcttctagaaaccttctgagtcacgaacagccaatttcttttgttactttgccatcatttaatgatcattcttttgttaatattactgtctctgcggcttttggtggtcttagtagtgccaatctcgttgcatctgacactactatcaatgggtttagttctttctgtgttgacactagacaatttaccattacactgttttataatgttacaaacagttatggttatgtgtctaaatcacaggatagtaattgtcctttcaccttgcaatctgttaatgattacctgtcttttagcaaattttgtgtttcaaccagccttttggctggtgcttgtaccatagatctttttggttaccctgcgttcggtagtggtgttaagttgacgtccctttattttcaattcacaaaaggtgagttgattactggcacgcctaaaccacttgaaggtatcacagacgtttcttttatgactctggatgtgtgtaccaagtatactatctatggctttaaaggtgagggtattattacccttacaaattctagcattttggcaggtgtttattatacatctgattctggacagttgttagcctttaagaatgtcactagtggtgctgtttattctgtcacgccatgttctttttcagagcaggctgcatatgttaatgatgatatagtgggtgttatttctagtttgtctaactccacttttaacaatactagggagttgcctggtttcttctaccattctaatgacggctccaattgtacagagcctgtgttggtgtatagtaacataggtgtttgtaaatctggcagtattggctatgttccatctcagtatggccaagtcaagattgcacccacggttactgggaatattagtattcccaccaactttagtatgagtattagaacagaatatttacagctttacaacacgcctgttagtgttgattgtgctacatatgtttgtaatggtaactctcgttgtaaacaattactcacccagtacactgcagcatgtaagaccatagagtcagcattacaactcagcgctaggcttgagtctgttgaagttaactctatgcttaccatttctgaagaggctttacagttagctaccatcagttcgtttaatggtgatggatataactttactaatgtgctgggtgcttccgtgtacgatcctgcaagtggcagggtggtacaaaaaaggtctgttattgaagacttgctttttaataaagtggttactaatggccttggtactgttgatgaagactataagcgctgttctaatggtcgctctgtggctgatctagtctgtgcgcagtattactctggtgtcatggtactacctggcgttgttgacgctgagaagcttcacatgtacagtgcgtctctcataggtggtatggcgctaggaggtataactgctgcagcggcattgccttttagctatgctgttcaagcgagactcaattatcttgctttacagacggatgttctacagcggaaccagcaattgcttgctgagtcttttaactctgctattggtaatataacttcagcctttgagagtgttaaagaggctattagtcaaacttccaagggtttgaacactgtggctcatgcgcttactaaggttcaagaggttgttaattcgcagggttcagctttgaaccaacttaccgtacagctgcaacacaacttccaagccatttctagttctattgatgacatttattcccgactggacattcttttagccgatgttcaggttgatcgtctcatcaccggcagattatcagcacttaatgcttttgttgcccaaaccctcactaagtatactgaggttcaggctagcaggaagctagcacagcaaaaggttaatgagtgcgtcaaatcgcaatctcagcgttacggtttttgtggtggtgatggcgagcacattttctctctggtacaggccgcacctcagggcctgctgttcttacatacagtacttgtaccgggtgattttgtaaatgttcttgccatcgctggcttatgcgttaatggtgaaattgccttgactctacgtgagcctggcttagtcttgtttacgcatgaacttcaaacttatactgcgacggaatattttgtttcatcgcgacgtatgtttgaacctagaaaacctaccgttagtgattttgttcaaattgagagttgtgtggtcacctatgtcaatctgactagcgaccagctaccagatgtaatcccagattacatcgatgttaacaaaacacttgatgagattttagcttctctgcccaatagaactggtccaagtcttcccctagatgtttttaatgccacttatcttaatcttactggtgaaattgcagatctagagcagcgttcagagtctctccgtaatactacagaagagctccgaagtctcattaacaacatcaacaacacacttgttgaccttgagtggctcaaccgagttgagacatacatcaagtggccgtggtgggtttggttgatcattgttattgttctcatctttgttgtgtcattactagtgttctgctgcatttccacgggttgttgtggatgctgcggttgctgcggtgcttgtttttcaggttgttgtaggggtcctagacttcaaccttacgaagcttttgaaaaggtccacgtgcagtga

SEQ ID NO: 3 - DNA, 4152 bp, Artificial Sequence
  DNA sequence encoding spike protein of PEDV strain CV777
atgaggtctttaatttacttctggttgctcttaccagtacttccaacactcagcctaccacaagatgtcactaggtgccagtctactactaactttaggcggttcttttcaaaatttaatgttcaggcacctgccgtcgtcgttttgggtggttacctacctagtatgaactcttctagctggtactgtggcacaggcattgaaactgctagtggcgttcatggtatttttctcagctacatcgattctggtcagggctttgagattggcatttcgcaagagccgtttgatcctagtggttaccagctttatttacataaggccactaatggtaacactaatgctattgcacgactgcgcatttgccagtttcccgataataaaacattgggccctactgttaatgatgttacaacaggtcgtaactgcctattcaacaaagccattccagcttatatgcgtgatggaaaagatattgttgtcggcataacatgggataatgatcgtgtcactgtttttgctgacaagatctatcatttttatcttaaaaatgattggtcccgcgttgcgacaagatgttacaatcgcagaagttgtgctatgcaatatgtttatacacctacctactacatgcttaatgttactagtgcaggtgaggatggcatttattatgaaccctgtacagctaattgcactggttacgctgccaatgtatttgccactgattccaatggccatataccagaaggttttagttttaataattggtttcttttatccaatgactccactttgttgcatggtaaagtggtttccaaccaacccttgttggtcaattgtcttttggccattcctaagatttatggactaggccaatttttctcattcaatcacacgatggatggcgtttgtaatggagctgctgtggatcgtgccccagaggctctgaggtttaatattaatgacacctccgtcattcttgctgaaggctcaattgtacttcatactgctttaggaacaaatctttcttttgtttgcagtaattcctcagatcctcatttagccatctttgccatacctctgggtgctactgaagtaccctactattgctttcttaaagtggatacttacaactccactgtttataaattcttggctgttttacctcctactgtcagggaaattgtcatcaccaagtatggtgatgtttatgtcaatgggtttggctatttgcatctcggtttgttggatgctgtcacaattaatttcactggtcatggcactgacgatgacgtttcaggtttctggaccatagcatcgactaattttgttgatgcactcatcgaggttcaaggaacttccattcagcgtattctttattgtgatgatcctgttagccaactcaagtgttctcaggttgcttttgaccttgacgatggtttttaccccatctcttctagaaaccttctgagtcacgaacagccaatttcttttgttactttgccatcatttaatgatcattcttttgttaatattactgtctctgcggcttttggtggtcttagtagtgccaatctcgttgcatctgacactactatcaatgggtttagttctttctgtgttgacactagacaatttaccattacactgttttataatgttacaaacagttatggttatgtgtctaaatcacaggatagtaattgtcctttcaccttgcaatctgttaatgattacctgtcttttagcaaattttgtgtttcaaccagccttttggctggtgcttgtaccatagatctttttggttaccctgcgttcggtagtggtgttaagttgacgtccctttattttcaattcacaaaaggtgagttgattactggcacgcctaaaccacttgaaggtatcacagacgtttcttttatgactctggatgtgtgtaccaagtatactatctatggctttaaaggtgagggtattattacccttacaaattctagcattttggcaggtgtttattatacatctgattctggacagttgttagcctttaagaatgtcactagtggtgctgtttattctgtcacgccatgttctttttcagagcaggctgcatatgttaatgatgatatagtgggtgttatttctagtttgtctaactccacttttaacaatactagggagttgcctggtttcttctaccattctaatgacggctccaattgtacagagcctgtgttggtgtatagtaacataggtgtttgtaaatctggcagtattggctatgttccatctcagtatggccaagtcaagattgcacccacggttactgggaatattagtattcccaccaactttagtatgagtattagaacagaatatttacagctttacaacacgcctgttagtgttgattgtgctacatatgtttgtaatggtaactctcgttgtaaacaattactcacccagtacactgcagcatgtaagaccatagagtcagcattacaactcagcgctaggcttgagtctgttgaagttaactctatgcttaccatttctgaagaggctttacagttagctaccatcagttcgtttaatggtgatggatataactttactaatgtgctgggtgcttccgtgtacgatcctgcaagtggcagggtggtacaaaaaaggtctgttattgaagacttgctttttaataaagtggttactaatggccttggtactgttgatgaagactataagcgctgttctaatggtcgctctgtggctgatctagtctgtgcgcagtattactctggtgtcatggtactacctggcgttgttgacgctgagaagcttcacatgtacagtgcgtctctcataggtggtatggcgctaggaggtataactgctgcagcggcattgccttttagctatgctgttcaagcgagactcaattatcttgctttacagacggatgttctacagcggaaccagcaattgcttgctgagtcttttaactctgctattggtaatataacttcagcctttgagagtgttaaagaggctattagtcaaacttccaagggtttgaacactgtggctcatgcgcttactaaggttcaagaggttgttaattcgcagggttcagctttgaaccaacttaccgtacagctgcaacacaacttccaagccatttctagttctattgatgacatttattcccgactggacattctttcagccgatgttcaggttgatcgtctcatcaccggcagattatcagcacttaatgcttttgttgcccaaaccctcactaagtatactgaggttcaggctagcaggaagctagcacagcaaaaggttaatgagtgcgtcaaatcgcaatctcagcgttacggtttttgtggtggtgatggcgagcacattttctctctggtacaggccgcacctcagggcctgctgttcttacatacagtacttgtaccgggtgattttgtaaatgttcttgccatcgctggcttatgcgttaatggtgaaattgccttgactctacgtgagcctggcttagtcttgtttacgcatgaacttcaaacttatactgcgacggaatattttgtttcatcgcgacgtatgtttgaacctagaaaacctaccgttagtgattttgttcaaattgagagttgtgtggtcacctatgtcaatctgactagcgaccagctaccagatgtaatcccagattacatcgatgttaacaaaacacttgatgagattttagcttctctgcccaatagaactggtccaagtcttcccctagatgtttttaatgccacttatcttaatcttactggtgaaattgcagatctagagcagcgttcagagtctctccgtaatactacagaagagctccgaagtctcattaacaacatcaacaacacacttgttgaccttgagtggctcaaccgagttgagacatacatcaagtggccgtggtgggtttggttgatcattgttattgttctcatctttgttgtgtcattactagtgttctgctgcatttccacgggttgttgtggatgctgcggttgctgcggtgcttgtttttcaggttgttgtaggggtcctagacttcaaccttacgaagcttttgaaaaggtccacgtgcagtga

SEQ ID NO: 4 - protein, 1383 aa, Artificial Sequence
  amino acid sequence of spike protein of PEDV strain Calaf14
MKSLNYFWLFLPVLSTLSLPQDVTRCQSTINFRRFFSKFNVQAPAVVVLGGYLPSMNSSSWYCGTGLETASGVHGIFLSYIDAGQGFEIGISQEPFDPSGYQLYLHKATNGNHNAIARLRICQFPNNKTLGPTVNDVTTGRNCLFNKAIPAYMQDGKNIVVGITWDNDRVTVFADKIYHFYLKNDWSRVATRCYNKRSCAMQYVYTPTYYMLNVTSAGEDGIYYEPCTANCSGYAANVFATDSNGHIPEGFSFNNWFLLSNDSTLLHGKVVSNQPLLVNCLLAIPKIYGLGQFFSFNQTMDGVCNGAAAQRAPEALRFNINDTSVILAEGSIVLHTALGTNLSFVCSNSSDPHLATFTIPLGATQVPYYCFLKVDTYNSTVYKFLAVLPPTVREIVITKYGDVYVNGFGYLHLGLLDAVTINFTGHGTDDDVSGFWTIASTNFVDALIEVQGTAIQRILYCDDPVSQLKCSQVAFDLDDGFYPISSRNLLSHEQPISFVTLPSFNDHSFVNITVSASFGGHSGANLIASDTTINGFSSFCVDTRQFTISLFYNVTNSYGYVSKSQDSNCPFTLQSVNDYLSFSKFCVSTNLLASDCTIDLFGYPEFGSGVKFTSLYFQFTKGELITGTPKPLEGVTDVSFMTLDVCTKYTIYGFKGEGIITLTNSSFLAGVYYTSDSGQLLAFKNVTSGAVYSVTPCSFSEQAAYVDDDIVGVISSLSSSTFNSTRELPGFFYHSNDGSNCTEPVLVYSNIGVCKSGSIGYVPSQSGQVKIAPTVTGNISIPTNFSMSIRTEYLQLYNTPVSVDCATYVCNGNSRCKQLLTQYTAACKTIESALQLSARLESVEVNSMLTISEEALQLATISSFNGDGYNFTNVLGVSVYDPASGRVVQKRSFIEDLLFNKVVTNGLGTVDEDYKRCSNGRSVADLVCAQYYSGVMVLPGVVDAEKLHMYSASLIGGMVLGGFTSAAALPFSYAVQARLNYLALQTDVLQRNQQLLAESFNSAIGNITSAFESVKEAISQTSKGLNTVAHALTKVQEVVNSQGAALTQLTVQLQHNFQAISSSIDDIYSRLDILSADVQVDRLITGRLSALNAFVAQTLTKYTEVQASRKLAQQKVNECVKSQSQRYGFCGGDGEHIFSLVQAAPQGLLFLHTVLVPGDFVDVIAIAGLCVNDEIALTLREPGLVLFTHELQNHTATEYFVSSRRMFEPRKPTVSDFVQIESCVVTYVNLTRDQLPDVIPDYIDVNKTLDEILASLPNRTGPSLPLDVFNATYLNLTGEIADLEQRSESLRNTTEELQSLIYNINNTLVDLEWLNRVETYIKWPWWVWLIIFIVLIFVVSLLVFCCISTGCCGCCGCCCACFSGCCRGPRLQPYEVFEKVHVQ

SEQ ID NO: 5 - protein, 1383 aa, Artificial Sequence
  amino acid sequence of spike protein of PEDV strain Br1-87
MRSLIYFWLLLPVLPTLSLPQDVTRCQSTTNFRRFFSKFNVQAPAVVVLGGYLPSMNSSSWYCGTGIETASGVHGIFLSYIDSGQGFEIGISQEPFDPSGYQLYLHKATNGNTNATARLRICQFPDNKTLGPTVNDVTTGRNCLFNKAIPAYMRDGKDIVVGITWDNDRVTVFADKIYHFYLKNDWSRVATRCYNRRSCAMQYVYTPTYYMLNVTSAGEDGIYYEPCTANCTGYAANVFATDSNGHIPEGFSFNNWFLLSNDSTLLHGKVVSNQPLLVNCLLAIPKIYGLGQFFSFNHTMDGVCNGAAVDRAPEALRFNINDTSVILAEGSIVLHTALGTNLSFVCSNSSDPHLAIFAIPLGATEVPYYCFLKVDTYNSTVYKFLAVLPSTVREIVITKYGDVYVNGFGYLHLGLLDAVTIYFTGHGTDDDVSGFWTIASTNFVDALIEVQGTSIQRILYCDDPVSQLKCSQVAFDLDDGFYPISSRNLLSHEQPISFVTLPSFNDHSFVNITVSAAFGGLSSANLVASDTTINGFSSFCVDTRQFTITLFYNVTNSYGYVSKSQDSNCPFTLQSVNDYLSFSKFCVSTSLLAGACTIDLFGYPAFGSGVKLTSLYFQFTKGELITGTPKPLEGITDVSFMTLDVCTKYTIYGFKGEGIITLTNSSILAGVYYTSDSGQLLAFKNVTSGAVYSVTPCSFSEQAAYVNDDIVGVISSLSNSTFNNTRELPGFFYHSNDGSNCTEPVLVYSNIGVCKSGSIGYVPSQYGQVKIAPTVTGNISIPTNFSMSIRTEYLQLYNTPVSVDCATYVCNGNSRCKQLLTQYTAACKTIESALQLSARLESVEVNSMLTISEEALQLATISSFNGDGYNFTNVLGASVYDPASGRVVQKRSVIEDLLFNKVVTNGLGTVDEDYKRCSNGRSVADLVCAQYYSGVMVLPGVVDAEKLHMYSASLIGGMALGGITAAAALPFSYAVQARLNYLALQTDVLQRNQQLLAESFNSAIGNITSAFESVKEAISQTSKGLNTVAHALTKVQEVVNSQGSALNQLTVQLQHNFQAISSSIDDIYSRLDILLADVQVDRLITGRLSALNAFVAQTLTKYTEVQASRKLAQQKVNECVKSQSQRYGFCGGDGEHIFSLVQAAPQGLLFLHTVLVPGDFVNVLAIAGLCVNGEIALTLREPGLVLFTHELQTYTATEYFVSSRRMFEPRKPTVSDFVQIESCVVTYVNLTSDQLPDVIPDYIDVNKTLDEILASLPNRTGPSLPLDVFNATYLNLTGEIADLEQRSESLRNTTEELRSLINNINNTLVDLEWLNRVETYIKWPWWVWLIIVIVLIFVVSLLVFCCISTGCCGCCGCCGACFSGCCRGPRLQPYEAFEKVHVQ

SEQ ID NO: 6 - protein, 1383 aa, Artificial Sequence
  amino acid sequence of spike protein of PEDV strain CV777
MRSLIYFWLLLPVLPTLSLPQDVTRCQSTTNFRRFFSKFNVQAPAVVVLGGYLPSMNSSSWYCGTGIETASGVHGIFLSYIDSGQGFEIGISQEPFDPSGYQLYLHKATNGNTNAIARLRICQFPDNKTLGPTVNDVTTGRNCLFNKAIPAYMRDGKDIVVGITWDNDRVTVFADKIYHFYLKNDWSRVATRCYNRRSCAMQYVYTPTYYMLNVTSAGEDGIYYEPCTANCTGYAANVFATDSNGHIPEGFSFNNWFLLSNDSTLLHGKVVSNQPLLVNCLLAIPKIYGLGQFFSFNHTMDGVCNGAAVDRAPEALRFNINDTSVILAEGSIVLHTALGTNLSFVCSNSSDPHLAIFAIPLGATEVPYYCFLKVDTYNSTVYKFLAVLPPTVREIVITKYGDVYVNGFGYLHLGLLDAVTINFTGHGTDDDVSGFWTIASTNFVDALIEVQGTSIQRILYCDDPVSQLKCSQVAFDLDDGFYPISSRNLLSHEQPISFVTLPSFNDHSFVNITVSAAFGGLSSANLVASDTTINGFSSFCVDTRQFTITLFYNVTNSYGYVSKSQDSNCPFTLQSVNDYLSFSKFCVSTSLLAGACTIDLFGYPAFGSGVKLTSLYFQFTKGELITGTPKPLEGITDVSFMTLDVCTKYTIYGFKGEGIITLTNSSILAGVYYTSDSGQLLAFKNVTSGAVYSVTPCSFSEQAAYVNDDIVGVISSLSNSTFNNTRELPGFFYHSNDGSNCTEPVLVYSNIGVCKSGSIGYVPSQYGQVKIAPTVTGNISIPTNFSMSIRTEYLQLYNTPVSVDCATYVCNGNSRCKQLLTQYTAACKTIESALQLSARLESVEVNSMLTISEEALQLATISSFNGDGYNFTNVLGASVYDPASGRVVQKRSVIEDLLFNKVVTNGLGTVDEDYKRCSNGRSVADLVCAQYYSGVMVLPGVVDAEKLHMYSASLIGGMALGGITAAAALPFSYAVQARLNYLALQTDVLQRNQQLLAESFNSAIGNITSAFESVKEAISQTSKGLNTVAHALTKVQEVVNSQGSALNQLTVQLQHNFQAISSSIDDIYSRLDILSADVQVDRLITGRLSALNAFVAQTLTKYTEVQASRKLAQQKVNECVKSQSQRYGFCGGDGEHIFSLVQAAPQGLLFLHTVLVPGDFVNVLAIAGLCVNGEIALTLREPGLVLFTHELQTYTATEYFVSSRRMFEPRKPTVSDFVQIESCVVTYVNLTSDQLPDVIPDYIDVNKTLDEILASLPNRTGPSLPLDVFNATYLNLTGEIADLEQRSESLRNTTEELRSLINNINNTLVDLEWLNRVETYIKWPWWVWLIIVIVLIFVVSLLVFCCISTGCCGCCGCCGACFSGCCRGPRLQPYEAFEKVHVQ

SEQ ID NO: 7 - DNA, 28062 bp, Artificial Sequence
  full DNA sequence corresponding to full length RNA of seed stock of PEDV virus USA/Colorado/2013, GenBank accession KF272920
tttttttttttcaagcagaagacggcatacgagattggtcagtgactggttcagacgtgtgctcttccgattttctatctacggatagttagctctttttctagactcttgtctactcaattcaactaaacgaaattttgtccttccggccgcatgtccatgctgctggaagctgacgtggaatttcattaggtttgcttaagtagccatcgcaagtgctgtgctgtcctctagttcctggttggcgttccgtcgccttctacatactagacaaacagccttcctccggttccgtctgggggttgtgtggataactagttctgtctagtttgaaaccagtaactgtcggctatggctagcaaccatgttacattggcttttgccaatgatgcagaaatttcagcttttggcttttgcactgctagtgaagccgtctcatactattctgaggccgccgctagtggatttatgcaatgccgtttcgtgtccttcgatctcgctgacactgttgagggattgcttcccgaagactatgtcatggtggtggtcggcactaccaagcttagtgcgtatgtggacacttttggtagccgccccaaaaacatttgtggttggctgttattttctaactgtaattacttcctcgaagagttagagcttacttttggtcgtcgtggtggtaacatcgtgccagttgaccaatacatgtgtggcgctgacggtaaacctgttcttcaggaatccgaatgggagtatacagatttctttgctgactccgaagacggtcaactcaacattgctggtatcacttatgtgaaggcctggattgtagagcgatcggatgtctcttatgcgagtcagaatttaacatctattaagtctattacttactgttcaacctatgagcatacttttcctgatggtactgccatgaaggttgcacgtactccaaagattaagaagactgttgtcttgtctgagccacttgctactatctacagggaaattggttctccttttgtggataatgggagcgatgctcgttctatcattaagagaccagtgttcctccacgcttttgttaagtgtaagtgtggtagttatcattggactgttggtgattggacttcctatgtctccacttgctgtggctttaagtgtaagccagtccttgtggcttcatgctctgctacgcctggttctgttgtggttacgcgcgctggtgctggcactggtgttaagtattacaacaacatgttcctgcgccatgtggcagacattgatgggttggcattctggcgaattctcaaggtgcagtccaaagacgacctcgcttgctctggtaaattccttgaacaccatgaggaaggtttcacagatccttgctactttttgaatgactcgagcattgctactaagctcaagtttgacatccttagtggcaagttttctgatgaagtcaaacaagctatctttgctggtcatgttgttgttggcagcgcgctcgttgacattgttgacgatgcactgggacagccttggtttatacgtaagcttggtgaccttgcaagtgcagcttgggagcagcttaaggctgtcgttagaggccttaacctcctgtctgatgaggtcgtgctctttggcaaaagacttagctgtgccactcttagtatcgttaacggtgtttttgagttcatcgccgaagtgcctgagaagttggctgcggctgttacagtttttgtcaacttcttgaatgagctttttgagtctgcctgtgactgcttaaaggtcggaggtaaaacctttaacaaggttggctcttatgttctttttgacaacgcattggttaagcttgtcaaggcaaaagttcgcggcccacgacaggcaggtgtttgtgaagttcgttacacaagccttgttattgggagtactaccaaggtggtttccaagcgcgttgaaaatgccaatgtgaatctcgtcgtcgttgacgaggatgtgaccctcaacaccactggtcgtacagttgttgttgacggacttgcattcttcgagagtgacgggttttacagacatcttgctgatgctgacgttgtcattgaacatcctgtttataagtctgcttgtgagctcaagccagtttttgagtgtgacccaatacctgattttcctatgcctgtggccgctagtgttgcagagctttgtgtgcaaactgatctgttgcttaaaaattacaacactccttataaaacttacagctgcgttgtgagaggtgataagtgttgtatcacttgcaccttacatttcacagcaccaagttatatggaggctgctgctaattttgtagacctctgtaccaagaacattggtactgctggttttcatgagttttacattacggcccatgaacaacaggatctgcaagggttcgtaaccacttgttgcacgatgtcaggttttgagtgttttatgcctataatcccacagtgtccagcagtgcttgaagagattgatggtggtagcatctggcggtcttttatcactggtcttaatacaatgtgggatttttgcaagcatcttaaagtcagctttggactagatggcattgttgtcactgtagcacgcaaatttaaacgacttggtgctctcttggcagaaatgtataacacttacctttcaactgtggtggaaaacttggtactggccggtgttagcttcaagtattatgccaccagtgtcccaaaaattgttttgggctgttgttttcacagtgttaaaagtgttcttgcaagtgccttccagattcctgtccaggcaggcgttgagaagtttaaagtcttccttaactgtgttcaccctgttgtaccacgtgtcattgaaacttcttttgtggaattagaagagacgacatttaaaccaccagcactcaatggtagtattgctattgttgatggctttgctttctattatgatggaacactatactatcccaccgatggtaatagcgttgttcctatctgctttaagaagaaaggtggtggtgatgtcaaattctctgatgaagtctctgttaaaaccattgacccagtttataaggtctcccttgaatttgagttcgagtctgagactattatggctgtgcttaataaggctgttggtaattgtatcaaggttacaggtggttgggacgatgttgttgagtatatcaatgttgccattgaggttcttaaagatcacatcgatgtgcctaagtactacatctatgatgaggaaggtggcaccgatcctaatctgcccgtaatggtttctcagtggccgttgaatgatgacacgatctcacaggatctgcttgatgttgaagttgttactgatgcgccagttgatttcgagggtgatgaagtagactcctctgaccctgataaggtggcagacgtggctaactctgagcctgaggatgacggtcttaatgtagctcctgaaacaaatgtagagtctgaagttgaggaagttgccgcaaccttgtcctttattaaagatacaccttccacagttactaaggatccttttgcttttgactttgcaagctatggaggacttaaggttttaagacaatctcataacaactgctgggttacttctaccttggtgcagctacaattgcttggcatcgttgatgaccctgcaatggagctttttagtgctggtagagttggtccaatggttcgcaaatgctatgagtcacaaaaggctatcttgggatctttgggtgatgtgtcggcttgcctagagtctctgactaaggacctacacacacttaagattacctgttctgtagtctgtggttgtggtactggtgaacgtatctatgatggttgtgcttttcgtatgacgccaactttggaaccgttcccatatggtgcttgtgctcagtgtgctcaagttttgatgcacacttttaaaagtattgttggcaccggcatcttttgtcgagatactactgctctctccttggattctttggttgtaaaacctctttgtgcggctgcttttataggcaaggatagtggtcattatgtcactaacttttatgatgctgctatggctattgatggttatggtcgtcatcagataaagtatgacacactgaacactatttgtgttaaagacgttaattggacagcaccttttgtcccagacgttgagcctgtattggagcttgttgtcaaacctttctattcttataagaatgttgatttttaccaaggagattttagtgaccttgttaaacttccatgtgattttgttgttaatgctgcaaatgagaatttgtctcacggtggcggcatagcaaaggccattgatgtttataccaagggcatgttgcagaagtgctcgaatgattacattaaagcacacggtcccattaaagttggacgtggtgtcatgttggaggcattaggtcttaaggtctttaatgttgttggtccacgtaagggtaagcatgcacctgagcttcttgttaaggcttataagtccgtttttgctaattcaggtgttgctcttacacctttgattagtgttggaatttttagtgttcctttggaagaatctttatctgcttttcttgcatgtgttggtgatcgccactgtaagtgcttttgttatagtgacaaagagcgcgaggcgatcattaattacatggatggcttggtagatgctattttcaaagatgcacttgttgatactactcctgtccaggaagatgttcaacaagtttcacaaaaaccagttttgcctaattttgaacctttcaggattgaaggtgctcatgctttctatgagtgcaaccctgaaggtttgatgtcattaggtgctgacaagctggtgttgtttacaaattccaatttggatttttgtagcgttggtaagtgtcttaacaatgtgactggcggtgcattgcttgaagccataaatgtatttaaaaagagtaacaaaacagtgcctgctggcaactgtgttacttttgagtgtgcagatatgatttctattactatggtagtattgccatctgacggtgatgctaattatgacaaaaattatgcacgcgccgtcgtcaaggtatctaagcttaaaggcaagttattgcttgctgttggtgatgccatgttgtattccaagttgtcccacctcagcgtgttaggtttcgtatccacacctgatgatgtggagcgtttctacgcaaataagagtgtggttattaaagttactgaggatacacgtagtgttaagactgttaaagtagaatccactgttacttatggacaacaaattggaccttgtcttgttaatgacaccgttgtcacagacaacaaacctgttgttgctgatgttgtagctaaggttgtaccaagtgctaattgggattcacattatggttttgataaggctggtgagttccacatgctagaccatactgggtttgcctttcctagtgaagttgttaacggtaggcgtgtgcttaaaaccacagataataactgttgggttaatgttacatgtttacaattacagtttgctagatttaggttcaagtcagcaggtctacaggctatgtgggagtcctattgtactggtgatgttgctatgtttgtgcattggttgtactggcttactggtgttgacaaaggtcagcctagtgattcagaaaatgcacttaacatgttgtctaagtacattgttcctgctggttctgtcactattgaacgtgtcacgcatgacggttgttgttgtagtaagcgtgttgtcactgcaccagttgtgaatgctagcgtgttgaagcttggcgtcgaggatggtctttgtccacatggtcttaactacattgacaaagttgttgtagttaaaggtactacaattgttgtcaatgttggaaaacctgtagtggcaccatcgcacctctttcttaagggtgtttcctacacaacattcctagataatggtaacggtgttgccggccattatactgtttttgatcatgacactggtatggtgcatgatggagatgtttttgtaccaggtgatctcaatgtgtctcctgttacaaatgttgtcgtctcagagcagacggctgttgtgattaaagaccctgtgaagaaagtagagttagacgctacaaagctgttagacactatgaattatgcatcggaaagattcttttcctttggtgattttatgtcacgtaatttaattacagtgtttttgtacatccttagtattttgggtctctgttttagggcctttcgtaagagggatgttaaagttctagctggtgtaccccaacgtactggtattatattgcgtaaaagtgtgcgctataatgcaaaggctttgggtgtcttcttcaagctaaaactttattggttcaaagttcttggtaagtttagtttgggtatttatgcattgtatgcattactattcatgacaatacgctttacacctataggtggccctgtttgtgatgatgttgttgctggttatgctaattctagttttgacaagaatgagtattgcaacagtgttatttgtaaggtctgtctctatgggtaccaggaactttcggacttctctcacacacaggtagtatggcaacaccttagagacccattaattggtaatgtgatgcctttcttttatttggcatttctggcaatttttgggggtgtttatgtaaaggctattactctctattttattttccagtatcttaacatacttggtgtgtttttgggcctacaacagtccatttggtttttgcagcttgtgccttttgatgtctttggtgacgagatcgtcgtctttttcatcgttacacgcgtattgatgttccttaagcatgttttccttggctgcgataaggcatcttgtgtggcttgctctaagagtgctcgccttaagcgcgttcctgtccagactatttttcagggtactagcaaatccttctacgtacatgccaatggtggttctaagttctgtaagaagcacaatttcttttgtttaaattgtgattcttatggtccaggctgcacttttattaatgacgtcattgcaactgaagttggtaatgttgtcaaacttaatgtgcaaccgacaggtcctgccactattcttattgacaaggttgaattcagtaatggtttttactatctttatagtggtgacacattttggaagtacaactttgacataacagataacaaatacacttgcaaagagtcacttaaaaattgtagcataatcacagactttattgtttttaacaataatggttccaatgtaaatcaggttaagaatgcatgtgtgtatttttcacagatgctttgtaaacctgttaagttagtggactcagcgttgttggccagtttgtctgttgattttggtgcaagcttacatagtgcttttgttagtgtgttgtcgaatagttttggcaaagacctgtcaagttgtaatgacatgcaggattgcaagagcacattgggttttgatgatgtaccattggatacctttaatgctgctgttgctgaggctcatcgttacgatgtcctcttgactgacatgtcgttcaacaattttaccaccagttatgcaaaaccagaggaaaaacttcccgtccatgacattgccacgtgtatgcgtgtaggtgccaagattgttaatcataacgttcttgtcaaggatagtatacctgtggtgtggcttgtacgtgatttcattgccctttctgaagaaactaggaagtacattattcgtacgactaaagttaagggtataaccttcatgttgacctttaatgattgtcgtatgcatactaccatacctactgtttgcattgcaaataagaagggtgcaggtcttcctagtttttcaaaggttaagaaattcttctggtttttgtgtctgttcatagttgctgttttctttgcactaagcttttttgattttagtactcaggttagcagtgatagtgattatgacttcaagtatattgagagtggccagttgaagacttttgacaatccacttagttgtgtgcataatgtctttagtaacttcgaccagtggcatgatgccaagtttggtttcacccccgtcaacaatcctagttgtcctatagtcgttggtgtatcagacgaagcgcgcactgttccaggtatcccagcaggtgtttatttagctggtaaaacacttgtttttgctattaacaccatttttggtacatctggtttgtgctttgatgctagtggcgttgctgataagggcgcttgcatttttaattcggcttgcaccacattatctggtttgggtggaactgctgtctactgttataagaatggtctagttgaaggtgctaaactttatagtgagttggcacctcatagctactataaaatggtagatggtaatgctgtgtctttacctgaaattatctcacgcggctttggcatccgtactatccgtacaaaggctatgacctactgtcgcgttggccagtgtgtgcaatctgcagaaggtgtttgttttggcgccgatagattctttgtctataatgcagaatctggttctgactttgtttgtggcacagggctctttacattgttgatgaacgttattagtgttttttccaagacagtaccagtaactgtgttgtctggtcaaatactttttaattgcattattgcttttgctgctgttgcggtgtgtttcttatttacaaagtttaagcgcatgttcggtgatatgtctgttggcgttttcactgtcggtgcttgtactttgttgaacaatgtttcctacattgtaacacagaacacacttggcatgttgggctatgcaactttgtactttttgtgcactaaaggtgttagatatatgtggatttggcatttgggatttttgatctcatatatacttattgcaccatggtgggttttgatggtttatgccttttcagccatttttgagtttatgcctaacctttttaagcttaaggtttcaacacaactttttgagggtgacaagttcgtaggctcttttgaaaatgctgcagcaggtacatttgtgcttgatatgcatgcctatgagagacttgccaactctatctcaactgaaaaactgcgtcagtatgctagtacttacaataagtacaagtattattcaggcagtgcttcagaggctgattacaggcttgcttgttttgcccatttggccaaggctatgatggattatgcttctaatcacaacgacacgttatacacaccacccactgtgagttacaattcaactctacaggctggcttgcgtaagatggcacaaccatctggtgttgttgagaagtgcatagttcgtgtttgctatggtaatatggctcttaatggcctatggcttggtgatactgttatctgcccacgccatgttatagcgtctagtactactagcactatagattatgactatgccctttctgttttacgcctccacaacttctccatttcatctggtaatgttttcctaggtgttgtgggtgtaaccatgcgaggtgctttgttgcagataaaggttaatcaaaacaatgtccacacgcctaagtacacctatcgcacagttagaccgggtgaatcttttaatatcttggcgtgctatgatggttctgcagctggtgtttacggcgttaacatgcgctctaattacactattagaggctcgttcattaatggcgcttgtggttcacctggttataacattaacaatggtaccgttgagttttgctatttacaccagcttgaacttggttcaggctgtcatgttggtagcgacttagatggtgttatgtatggtggttatgaggaccaacctactttgcaagttgaaggcgctagtagtctgtttacagagaatgtgttggcatttctttatgcagcactcattaatggttctacctggtggcttagttcttctaggattgctgtagacaggtttaatgagtgggctgttcataatggtatgacaacagtagttaatactgattgcttttctattcttgctgctaagactggtgttgatgtacaacgtttgttggcctcaatccagtctctgcataagaattttggtggaaagcaaattcttggctatacctcgttgacagatgagtttactacaggtgaagttatacgtcaaatgtatggcgttaatcttcagagtggttatgtttcacgcgcctgtagaaatgtcttgctggttggttcttttctgactttcttttggtcagaattagtttcctacactaagttcttttgggtaaatcctggttatgtcacacctatgtttgcgtgtttgtcattgctgtcctcacttttgatgttcacactcaagcataagacattgtttttccaggtctttctaatacctgctctgattgttacatcttgcattaatttggcatttgatgttgaagtctacaactatttggcagagcattttgattaccatgtttctctcatgggttttaatgcacaaggtcttgttaacatctttgtctgctttgttgttaccattttacacggcacatacacatggcgcttttttaacacacctgtgagttctgtcacttatgtggtagctttgctgactgcggcatataactatttttacgctagtgacattcttagttgtgctatgacactatttgctagtgtgactggcaactggttcgttggtgctgtttgttataaagctgctgtttatatggccttgagatttcctacttttgtggctatttttggtgatattaagagtgttatgttctgttaccttgtgttgggttattttacctgttgcttctacggtattctctactggttcaacaggttttttaaggttagtgtaggtgtctatgactatactgttagtgctgctgagtttaagtatatggttgctaacggcctacgtgcaccaactggaacacttgattcactacttctgtctgccaaattgattggtattggtggtgagcggaatattaagatttcttccgttcagtctaaactgactgatattaagtgtagtaacgttgtgcttttaggctgtctctctagcatgaatgtctcagcaaattcaacagaatgggcctattgtgttgacttgcataacaagatcaacttgtgtaatgacccagaaaaagcgcaggaaatgctacttgctttgttggcatttttccttagtaagaatagtgcttttggtttagatgacttattggaatcctattttaatgacaatagtatgttgcagagtgttgcatctacttatgtcggtttgccttcttatgtcatttatgaaaatgcacgccaacagtatgaagatgctgttaataatggttctccacctcagttggttaagcaattgcgccatgccatgaatgtagcaaagagcgaatttgaccgtgaggcttctactcagcgtaagcttgatagaatggcggaacaggctgcagcacagatgtacaaagaggcacgagcagttaataggaagtccaaagttgtaagtgctatgcattcactgctttttggtatgttgagacgtttggacatgtcttctgtagacaccattctcaacttggcaaaggatggggttgtacctctgtctgtcataccggcagtcagtgctactaagcttaacattgttacttctgatatcgattcttataatcgtatccagcgtgagggatgtgtccactacgctggtaccatttggaatataattgatatcaaggacaatgatggcaaggtggtacacgttaaggaggtaaccgcacagaatgctgagtccctgtcatggcccctggtccttgggtgtgagcgtattgtcaagctccagaataatgaaattattcccggtaagctgaagcagcgctccattaaggcagaaggagatggcatagttggagaaggtaaggcactttacaataatgagggtggacgtacttttatgtatgctttcatctcggacaaaccggacctgcgtgtagtcaagtgggagttcgatggtggttgtaacactattgagctagaaccaccacgtaagttcttggtggattctcctaatggtgcacagatcaagtatctctactttgttcgtaaccttaacacgttacgtaggggtgctgttctcggctacataggtgccactgtacgcttgcaggctggtaaacaaacagaacaggctattaactcttcattgttgacactttgcgctttcgctgtggatcctgctaagacctacatcgatgctgtcaaaagtggtcacaaaccagtaggtaactgtgttaagatgttggccaatggttctggtaatggacaagctgttactaatggtgtggaggctagtactaaccaggattcatacggtggtgcgtccgtgtgtctatattgtagagcacatgttgagcatccatctatggatggtttttgcagactgaaaggcaagtacgtacaggttccactaggtacagtggatcctatacgttttgtacttgagaatgacgtttgcaaggtttgtggttgttggctggctaatggctgcacttgtgacagatccattatgcaaagcactgatatggcttatttaaacgagtacggggctctagtgcagctcgactagagccctgtaacggtactgatacacaacatgtgtatcgtgcttttgacatctacaacaaggatgttgcttgtctaggtaaattcctcaaggtgaactgtgttcgcctgaagaatttggataagcatgatgcattctatgttgtcaaaagatgtaccaagtctgcgatggaacacgagcaatccatctatagcagacttgaaaagtgtggagccgtagccgaacacgatttcttcacttggaaggatggtcgtgccatctatggtaacgtttgtagaaaggatcttaccgagtatactatgatggatttgtgttacgctttacgtaactttgatgaaaacaattgcgatgttcttaagagcattttaattaaggtaggcgcttgtgaggagtcctacttcaataataaagtctggtttgaccctgttgaaaatgaagacattcatcgtgtctatgcattgttaggtaccattgtttcacgtgctatgcttaaatgcgttaagttctgtgatgcaatggttgaacaaggtatagttggtgttgtcacattagataatcaggatcttaatggtgatttttatgattttggtgattttacttgtagcatcaagggaatgggtatacccatttgcacatcatattactcttatatgatgcctgttatgggtatgactaattgccttgctagtgagtgttttgttaagagtgatatatttggtgaggatttcaagtcatatgacctgctggaatatgatttcacggagcataagacagcactcttcaacaagtatttcaagtattggggactgcaataccaccctaactgtgtggactgcagtgatgagcagtgcatagttcactgtgccaacttcaatacgttgttttccactactatacctattacggcatttggacctttgtgtcgcaagtgttggattgatggtgttccactggtaactacagctggttatcattttaaacagttaggtatagtttggaacaatgacctcaacttacactctagcaggctctctattaacgaattactccagttttgtagtgatcctgcattgcttatagcatcatcaccagcccttgttgatcagcgtactgtttgcttttcagttgcagcgctaggtacaggtatgactaaccagactgttaaacctggccatttcaataaggagttttatgacttcttacttgagcaaggtttcttttctgagggctctgagcttactttaaagcacttcttctttgcacagaagggtgatgcagctgttaaggattttgactactataggtataatagacctactgttctggacatttgccaagctcgcgtcgtgtatcaaatagtgcaacgctattttgatatttacgaaggtggttgtatcactgctaaagaggtggttgttacaaaccttaacaagagcgcaggttatcctttgaacaagtttggtaaagctggtctttactatgagtctttatcctatgaggaacaggatgaactttatgcttatactaagcgtaacatcctgcccactatgacacagctcaaccttaaatatgctataagtggcaaagaacgtgcacgcacagtgggtggtgtttcgcttttgtcaaccatgactactcggcagtatcatcagaaacaccttaagtccatagttaatactaggggcgcttcggttgttattggtactactaagttttatggtggttgggacaatatgcttaagaaccttattgatggtgttgaaaatccgtgtcttatgggttgggactacccaaagtgcgacagagcactgcccaatatgatacgtatgatttcagccatgattttaggctctaagcacaccacatgctgcagttccactgaccgctttttcaggttgtgcaatgaattggctcaagtccttactgaggttgtttattctaatggaggtttttatttgaagccaggtggtactacctctggtgatgcaaccaccgcatatgcaaactcagtttttaatatcttccaagcagtaagtgccaatgttaacaaacttcttagtgttgacagcaatgtctgtcataatttagaagttaagcaattgcagcgtaagctttatgagtgctgttatagatcaactaccgtcgatgaccagttcgtcgttgagtattatggttacttgcgtaaacatttttcaatgatgattctttctgatgatggcgttgtttgttataacaatgactatgcatcacttggttatgtcgctgatcttaacgcattcaaggctgttttgtattaccagaacaatgtcttcatgagcgcctctaaatgttggatcgagcctgacattaataaaggtcctcatgaattttgctcgcagcatactatgcagattgtcgataaagatggtacttattaccttccttaccctgatccttcaagaattctctctgcaggtgtgtttgttgatgacgttgttaaaactgatgcagttgtattgcttgaacgttatgtgtcattggctatagatgcctacccgttatctaagcatgaaaaccctgaatataagaaggtgttttatgtgcttttggattgggttaagcatctgtacaaaactcttaatgctggtgtgttagagtctttttctgtcacacttttggaagattctactgctaaattctgggatgagagcttttatgccaacatgtatgagaaatctgcagttttacaatctgcagggctttgtgttgtttgtggctctcaaactgttttacgttgtggtgattgtctacggcgtcctatgctttgtactaagtgtgcttatgatcatgtcattggaacaactcacaagttcattttggccatcactccatatgtgtgttgtgcttcagattgtggtgtcaatgatgtaactaagctctacttaggtggtcttagttattggtgtcatgaccacaagccacgtcttgcattcccgttgtgctctgctggtaatgtttttggcttgtacaaaaattctgctaccggctcacccgatgttgaagactttaatcgcattgctacatccgattggactgatgtttctgactacaggttggcaaatgatgtcaaggactcattgcgtctgtttgcagcggaaactatcaaggccaaggaggagagcgttaagtcatcctatgcttgtgcaacactacatgaggttgtaggacctaaagagttgttgctcaaatgggaagtcggcagacccaaaccaccccttaatagaaattcggttttcacttgttatcatataacgaagaacaccaaatttcaaatcggtgagtttgtgtttgagaaggcagaatatgataatgatgctgtaacatataaaactaccgccacaacaaaacttgttcctggcatggtttttgtgcttacctcacataatgttcagccattgcgcgcaccgaccattgctaatcaagaacgttattccactatacataagttgcatcctgcttttaacatacctgaagcttattctagcttagtgccctattaccaattgattggtaagcagaagattacaactattcagggacctcccggtagtggtaaatctcactgtgttatagggctaggtttgtactatccaggtgcacgtatagtgtttacagcttgttctcatgcagcggtcgattcactttgtgtgaaagcttccactgcttatagcaatgacaaatgttcacgcatcataccacagcgcgctcgtgttgagtgttatgatggtttcaagtctaataatactagtgctcagtaccttttctctactgtcaatgctttgccagagtgcaatgcggacattgttgtggtggatgaggtctctatgtgcactaattatgacttgtctgtcataaatcagcgcatcagctataggcatgtagtctatgttggtgaccctcaacagctgcctgcaccacgtgttatgatttcacgtggtactttggaaccaaaggactacaacgttgtcactcaacgcatgtgtgcccttaagcctgatgttttcttgcacaagtgttatcgctgtcctgctgagatagtgcgtactgtgtctgagatggtctatgaaaaccaattcattcctgtgcacccagatagcaagcagtgttttaaaatcttttgcaagggtaatgttcaggttgataatggttcaagcattaatcgcaggcaattggatgttgtgcgtatgtttttggctaaaaatcctaggtggtcaaaggctgtttttatttctccttataacagccagaattatgttgccagccgcatgctaggtctacaaattcagacagttgactcatcccagggtagtgagtatgactatgtcatttacacacaaacttcagatactgcccatgcctgtaatgttaacaggtttaatgttgccatcacaagggccaagaaaggcatattatgtataatgtgcgataggtccctttttgatgtgcttaaattctttgagcttaaattgtctgatttgcaggctaatgagggttgtggtctttttaaagactgtagcagaggtgatgatctgttgccaccatctcacgctaacaccttcatgtctttagcggacaattttaagactgatcaagatcttgctgttcaaataggtgttaatggacccattaaatatgagcatgttatctcgtttatgggtttccgttttgatatcaacatacccaaccatcatactctcttttgcacacgcgactttgccatgcgcaatgttagaggttggttaggctttgacgttgaaggagcacatgttgttggctctaacgtcggtacaaatgtcccattgcaattagggttttctaacggtgttgattttgttgtcagacctgaaggttgcgttgtaacagagtctggtgactacattaaacccgtcagagctcgtgctccaccaggggaacaattcgcacaccttttgcctttacttaaacgcggccaaccatgggatgttgtccgcaaacgtatagtgcagatgtgtagtgactacctggccaacctatcagacatactaatttttgtgttgtgggctggtggtttggagttgacaactatgcgttattttgtcaagattggaccaagtaagagttgtgattgtggtaaggttgctacttgttacaatagtgcgctgcatacgtactgttgtttcaaacatgcccttggttgtgattatctgtataacccatactgtattgatatacagcagtggggatacaagggatcacttagccttaaccaccatgagcattgtaatgtacatagaaacgagcatgtggcttctggtgatgccataatgactcgctgtctggccatacatgattgctttgtcaagaacgttgactggtccatcacatacccatttattggtaatgaggctgttattaataagagcggccgaattgtgcaatcacacactatgcggtcagttcttaagttatacaatccgaaagccatatatgatattggcaatcctaagggcattagatgtgccgtaacggatgctaagtggttttgctttgacaagaatcctactaattctaatgtcaagacattggagtatgactatataacacatggccaatttgatgggttgtgcttgttttggaattgcaatgtagacatgtatccagaattttctgtggtctgtcgttttgatactcgctgtaggtcaccactcaacttggagggttgtaatggtggttcactgtatgttaataatcatgcattccatacaccggcttttgacaagcgtgcttttgctaagttgaagccaatgccatttttcttttatgatgatactgagtgtgacaagttacaggactccataaactatgttcctcttagggctagtaactgcattactaaatgtaatgttggtggtgctgtctgtagtaagcattgtgctatgtatcatagctatgttaatgcttacaacacttttacgtcggcgggctttactatttgggtgcctacttcgtttgacacctataatctgtggcagacatttagtaacaatttgcaaggtcttgagaacattgctttcaatgtcgtaaagaaaggatcttttgttggtgccgaaggtgaacttcctgtagctgtggttaatgacaaagtgctcgttagagatggtactgttgatactcttgtttttacaaacaagacatcactacccactaacgtagcttttgagttgtatgccaagcgtaaggtaggactcaccccacccattacgatcctacgtaacttgggtgtagtttgtacatctaagtgtgtcatttgggactatgaagccgaacgtccacttactacttttacaaaggatgtttgtaaatataccgactttgagggtgacgtctgtacactctttgataacagcattgttggttcattagagcgattctccatgacccaaaatgctgtgcttatgtcacttacagctgttaaaaagcttactggcataaagttaacttatggttatcttaatggtgtcccagttaacacacatgaagataaaccttttacttggtatatttacactaggaagaacggcaagttcgaggaccatcctgatggctattttacccaaggtagaacaaccgctgattttagccctcgtagcgacatggaaaaggacttcctaagtatggatatgggtctgtttattaacaagtacggacttgaagattacggctttgagcacgttgtgtatggtgatgtttcaaaaaccacccttggtggtttgcatctactaatttcgcaggtgcgtctggcctgtatgggtgtgctcaaaatagacgagtttgtgtctagtaatgatagcacgttaaagtcttgtactgttacatatgctgataaccctagtagtaagatggtttgtacgtatatggatctcctgcttgacgattttgtcagcattcttaaatctttggatttgggcgttgtatctaaagttcatgaagttatggtcgattgtaaaatgtggaggtggatgttgtggtgtaaggatcataaactccagacattttatccgcaacttcaggccagtgaatggaagtgtggttattccatgccttctatttacaagatacaacgtatgtgtttagaaccttgcaatctctacaactatggtgctggtattaagttacctgatggcattatgtttaacgtagttaaatacacacagctttgtcaatatctcaatagcaccacaatgtgtgtaccccatcacatgcgtgtgctacatcttggtgctggctccgacaagggtgttgcacctggcacggctgtcttacgacgttggttgccactggatgccattatagttgacaatgatagtgtggattacgttagcgatgctgattatagtgttacaggagattgctctaccttatacctgtcagataagtttgatttagttatatctgatatgtatgatggtaagattaaaagttgtgatggggagaacgtgtctaaagaaggcttctttccctatattaatggtgtcatcaccgaaaagttggcacttggtggtactgtagctattaaggtgacggagtttagttggaataagaagttgtatgaactcattcagaggtttgagtattggacaatgttctgtaccagtgttaacacgtcatcgtcagaggcattcttaattggtgttcactatttaggtgattttgcaagtggcgctgtgattgacggcaacactatgcatgccaattatatcttctggcgtaattccacaattatgactatgtcttacaatagtgtacttgatttaagcaagttcaattgtaagcataaggctacagttgtcattaatttaaaagattcatccattagtgatgttgtgttaggtttgttgaagaatggtaagttgctagtgcgtaataatgacgccatttgtggtttttctaatcatttggtcaacgtaaacaaatgaagtctttaacctacttctggttgttcttaccagtactttcaacacttagcctaccacaagatgtcaccaggtgctcagctaacactaattttaggcggttcttttcaaaatttaatgttcaggcgcctgcagttgttgtactgggcggttatctacctattggtgaaaaccagggtgtcaattcaacttggtactgtgctggccaacatccaactgctagtggcgttcatggtatctttgttagccatattagaggtggtcatggctttgagattggcatttcgcaagagccttttgaccctagtggttaccagctttatttacataaggctactaacggtaacactaatgctactgcgcgactgcgcatttgccagtttcctagcattaaaacattgggccccactgctaataatgatgttacaacaggtcgtaattgcctatttaacaaagccatcccagctcatatgagtgaacatagtgttgtcggcataacatgggataatgatcgtgtcactgtcttttctgacaagatctattatttttattttaaaaatgattggtcccgtgttgcgacaaagtgttacaacagtggaggttgtgctatgcaatatgtttacgaacccacctattacatgcttaatgttactagtgctggtgaggatggtatttcttatcaaccctgtacagctaattgcattggttatgctgccaatgtatttgctactgagcccaatggccacataccagaaggttttagttttaataattggtttcttttgtccaatgattccactttggtgcatggtaaggtggtttccaaccaaccattgttggtcaattgtcttttggccattcctaagatttatggactaggccaatttttctcctttaatcaaacgatcgatggtgtttgtaatggagctgctgtgcagcgtgcaccagaggctctgaggtttaatattaatgacatctctgtcattcttgctgaaggctcaattgtacttcatactgctttaggaacaaatttttcttttgtttgcagtaattcctcaaatcctcacttagccaccttcgccatacctctgggtgctacccaagtaccttattattgtttttttaaagtggatacttacaactccactgtttataaatttttggctgttttacctcctaccgtcagggaaattgtcatcaccaagtatggtgatgtttatgtcaatgggtttggatacttgcatctcggtttgttggatgctgtcacaattaatttcactggtcatggcactgacgatgatgtttctggtttttggaccatagcatcgactaattttgttgatgcactcatcgaagttcaaggaaccgccattcagcgtattctttattgtgatgatcctgttagccaactcaagtgttctcaggttgcttttgaccttgacgatggtttttacactatttcttctagaaaccttctgagtcatgaacagccaatttcttttgttactctgccatcatttaatgatcattcttttgttaacattactgtatctgcttcctttggtggtcatagtggtgccaaccttattgcatctgacactactatcaatgggtttagttctttctgtgttgacactagacaatttaccatttcactgttttataacgttacaaacagttatggttatgtgtctaaatcacaggacagtaattgccctttcaccttgcaatctgttaatgattacctgtcttttagcaaattttgtgtttccaccagccttttggctagtgcctgtaccatagatctttttggttaccctgagtttggtagtggtgttaagtttacgtccctttactttcaattcacaaagggtgagttgattactggcacgcctaaaccacttgaaggtgtcacggacgtttcttttatgactctggatgtgtgtaccaagtatactatctatggctttaaaggtgagggtatcattacccttacaaattctagctttttggcaggtgtttattacacatctgattctggacagttgttagcctttaagaatgtcactagtggtgctgtttattctgttacgccatgttctttttcagagcaggctgcatatgttgatgatgatatagtgggtgttatttctagtttgtctagctccacttttaacagtactagggagttgcctggtttcttctaccattctaatgatggctctaattgtacagagcctgtgttggtgtatagtaacataggtgtttgtaaatctggcagtattggctacgtcccatctcagtctggccaagtcaagattgcacccacggttactgggaatattagtattcccaccaactttagtatgagtattaggacagaatatttacagctttacaacacgcctgttagtgttgattgtgccacatatgtttgtaatggtaactctcgttgtaaacaattactcacccagtacactgcagcatgtaagaccatagagtcagcattacaactcagcgctaggcttgagtctgttgaagttaactctatgcttactatttctgatgaggctctacagttagctaccattagttcgtttaatggtgatggatataattttactaatgtgctgggtgtttctgtgtatgatcctgcacgtggcagggtggtacaaaaaaggtcttttattgaagacctgctttttaataaagtggttactaatggccttggtactgttgatgaagactataagcgctgttctaatggtcgctctgtggcagatctagtctgtgcacagtattactctggtgtcatggtactacctggtgttgttgacgctgagaagcttcacatgtatagtgcgtctctcatcggtggtatggtgctaggaggttttacttctgcagcggcattgccttttagctatgctgttcaagctagactcaattatcttgctctacagacggatgttctacagcggaaccagcaattgcttgctgagtcttttaactctgctattggtaatataacttcagcctttgagagtgttaaagaggctattagtcaaacttccaagggtttgaacactgtggctcatgcgcttactaaggttcaagaggttgttaactcgcagggtgcagctttgactcaacttaccgtacagctgcaacacaacttccaagccatttctagttctattgatgacatttactctcgactggacattctttcagccgatgctcaggttgaccgtctcatcaccggcagattatcagcacttaatgcttttgttgctcaaaccctcactaagtatactgaggttcaggctagcaggaagttagcacagcaaaaggttaatgagtgcgttaaatcgcaatctcagcgttatggtttttgtggtggtgatggcgagcacattttctctctggtacaggcagcacctcagggcctgctgtttttacatacagtacttgtaccgagtgattttgtagatgttattgccatcgctggcttatgcgttaacgatgaaattgccttgactctacgtgagcctggcttagtcttgtttacgcatgaacttcaaaatcatactgcgacggaatattttgtttcatcgcgacgtatgtttgaacctagaaaacctaccgttagtgattttgttcaaattgagagttgtgtggtcacctatgtcaatttgactagagaccaactaccagatgtaatcccagattacatcgatgttaacaaaacacttgatgagattttagcttctctgcccaatagaactggtccaagtcttcctttagatgtttttaatgccacttatcttaatctcactggtgaaattgcagatttagagcagcgttcagagtctctccgtaatactacagaggagctccaaagtcttatatataatatcaacaacacactagttgaccttgagtggctcaaccgagttgagacatatatcaagtggccgtggtgggtttggttgattattttcattgttctcatctttgttgtgtcattactagtgttctgctgcatttccacgggttgttgtggatgctgcggctgctgctgtgcttgtttctcaggttgttgtaggggtcctagacttcaaccttacgaagtttttgaaaaggtccacgtgcagtgatgtttcttggactttttcaatacacgattgacacagttgtcaaagatgtctcaaagtctgctaacttgtctttggatgctgtccaagagttggagctcaatgtagttccaattagacaagcttcaaatgtgacgggttttcttttcaccagtgtttttatctacttctttgcactgtttaaagcgtcttctttgaggcgcaattatattatgttggcagcgcgttttgctgtcattgtttagatgcaactattatttgttgcacacttattggcaggctttgtttagtctgcttttactcctggcgctataaaaatgcgctctttattatttttaatactacgacactttctttcctcaatggtaaagcagcttatgacggcaaatccattgtgattttagaaggtggtgaccattacatcacttttggcaactcttttgttgcttttgttagtagcatcgacttgtatctagctatacgtgggcggcaagaagctgacctacagctgttgcgaactgttgagcttcttgatggcaagaagctttatgtcttttcgcaacatcaaattgttggcattactaatgctgcatttgactcaattcaactagacgagtatgctacaattagtgaatgataatggtctagtagttaatgttatactttggcttttcgtactctttttcctgcttattataagcattactttcgtccaattggttaatctgtgcttcacttgtcaccggttgtgtaatagcgcagtttacacacctatagggcgtttgtatagagtttataagtcttacatgcaaatagaccccctccctagtactgttattgacgtataaacgaaatatgtctaacggttctattcccgttgatgaggtgattcaacaccttagaaactggaatttcacatggaatatcatactgacgatactacttgtagtgcttcagtatggccattacaagtactctgcgttcttgtatggtgtcaagatggctattctatggatactttggcctcttgtgttagcactgtcactttttgatgcatgggctagctttcaggtcaattgggtcttttttgctttcagcatccttatggcttgcatcactcttatgctgtggataatgtactttgtcaatagcattcggttgtggcgcaggacacattcttggtggtctttcaatcctgaaacagacgcgcttctcactacttctgtgatgggccgacaggtctgcattccagtgcttggagcaccaactggtgtaacgctaacactccttagtggtacattgcttgtagagggctataaggttgctactggcgtacaggtaagtcaattacctaatttcgtcacagtcgccaaggccactacaacaattgtctacggacgtgttggtcgttcagtcaatgcttcatctggcactggttgggctttctatgtccggttcaaacacggcgactactcagctgtgagtaatccgagttcggttctcacagatagtgagaaagtgcttcatttagtctaaacagaaactttatggcttctgtcagttttcaggatcgtggccgcaaacgggtgccattatccctctatgcccctcttagggttactaatgacaaacccctttctaaggtacttgcaaataatgctgtacccactaataaaggaaataaggaccagcaaattggatactggaatgagcaaattcgctggcgcatgcgccgtggtgagcgaattgaacaaccttccaattggcatttctactacctcggaacaggacctcacgccgacctccgctataggactcgtactgagggtgttttctgggttgctaaagaaggcgcaaagactgaacccactaacctgggtgtcagaaaggcgtctgaaaagccaattattccaaatttctctcaacagcttcccagcgtagttgagattgttgaacctaacacacctcctacttcacgtgcaaattcacgtagcaggagtcgtggtaatggcaacaacaggtccagatctccaagtaacaacagaggcaataaccagtcccgcggtaattcacagaatcgtggaaataaccagggtcgtggagcttctcagaacagaggaggcaataataataacaataacaagtctcgtaaccagtccaagaacagaaaccagtcaaatgaccgtggtggtgtaacatcacgcgatgatctggtggctgctgtcaaggatgcccttaaatctttgggtattggcgaaaaccctgacaagcttaagcaacagcagaagcccaaacaggaaaggtctgacagcagcggcaaaaatacacctaagaagaacaaatccagagccacttcgaaagaacgtgacctcaaagacatcccagagtggaggagaattcccaagggcgaaaatagcgtagcagcttgcttcggacccaggggaggcttcaaaaattttggagatgcggaatttgtcgaaaaaggtgttgatgcctcaggctatgctcagatcgccagtttagcaccaaatgttgcagcattgctctttggtggtaatgtggctgttcgtgagctagcggactcttacgagattacatataattataaaatgactgtgccaaagtctgatccaaatgtagagcttcttgtttcacaggtggatgcatttaaaactgggaatgcaaaaccccagagaaagaaggaaaagaagaacaagcgtgaaaccacgcagcagctgaatgaagaggccatctacgatgatgtgggtgtgccatctgatgtgactcatgccaatttggaatgggacacagctgttgatggtggtgacacggccgttgaaattatcaacgagatcttcgacacaggaaattaaacaatgtttgactggcttatcctggctatgtcccagggtagtgccattacactgttattactgagtgtttttctagcgacttggctgctgggctatggctttgccctctaactagcggtcttggtcttgcacacaacggtaagccagtggtaatgtcagtgcaagaaggatattaccatagcactgtcatgaggggaacgcagtaccttttcatctaaacctttgcacgagtaatcaaagatccgcttgacgagcctatatggaagagcgtgccaggtatttgactcaaggactgttagtaactgaagacctgacggtgttgatatggatacacaaaaa

SEQ ID NO: 8 - DNA, 46 bp, Artificial Sequence
  oligonucleotide primer
atcgaccacatggctccaacacaccagtcgttaagcatggcaagct

SEQ ID NO: 9 - DNA, 21 bp, Artificial Sequence
  oligonucleotide primer
cagctcttgcccatgtagctt

SEQ ID NO: 10 - DNA, 27 bp, Artificial Sequence
  oligonucleotide probe
cacaccagtcgttaagcatggcaagct

SEQ ID NO: 11 - DNA, 25406 bp, Artificial Sequence
  full length encoding DNA for PDCoV virus USA/Indiana/2014/8501010
aaaattatagcattagtctataattttatctccctagcttcgctagttctctaccgacaccaatccaggtgcgtctgccaccaagttggctacccttcctaggggcgctttcgcgcttgctcaccattagattacctggaaaccagccattcaggttggagtttccccaggctcttttgtgtgggcattagcggcttgtggtttttgcacaaaatctaagctacttaccgttcctctgaccatccaccacttctatagacagcactgattaccgtagggtttaagtcacaccggtctgcaccgcccgtcagcggacacattacccagcatagcactccttgcaccgagcctaggtaggataaaaccccctaccgggtgactcttaaggcgtttcctccacgggatagccactagtcactaggtgtaagtgatctgatctgggcgtattgtgttgcgcaagtgtgatacccataggagcgtggaatcctattctgcggctcagtgcctgatatagctgtgaaatggccaagaacaagtccaagcgcgacgctattgcgttgcctgaaaatgtaccaccacctctgcaacttttcattcatgttgcagctgctgaagagggtcaccctaaggttactacttaccttggcaactataacctctatgccaccaaggctccgcctggcgtgcaggttcttagtgctaaaacctctcttactgactttgagaatgtctttggagctcaacccaccttgcgatcaattcgtaatctggtttgtgaggctcgctcggctgaatggacaacttccaagaatgcttttgcactcaaagccactcaacttgactactctgatgccgttttgagggcaatgattcgtttctgccctccaaaggtgtccacactcgctgcctttgctctttttggccgattggttaaaattgaggacaaggaacttgctgagttagctcgtgatactgcccttgagttggcgtacacggctaaaattggtacatctcttgctgacacgagatctgtctcacttattcataaggatgcttatctaactctcagtaatgaggttgttggcgtaacttttactgccgcacttatggcaaaggctaccactgttaatggagcaatgcaatactcaaacttttacctctaccctcgtgccactattaaggtaaccgatggtaaggctgaagcaattgcaactaagcctctgtctgctgccactaaaggcaagcaaatcacagaggatgtcaaccttctccctgactatcagcagctgcttgttgatcaagtgactggcactgaggttaaggttggagctctaacctatgttaagaccactgattcgccacccctttactttcccaaagtcaagggtggtgttattggtattgcacttaagcagcagggcactgcggctaagaagctcaatgtagtcttccatgctcaacctgatgatgttctgctagccttcatacaacttcagcaattcttgaaccgtacttcggattcaagtgttgaaattactgattgccagagttatgaagtatctccaactgtgacggtcaaaattggcccgtctaaacctggggatgtcatcgtggctactgatgaggaataccttaaatgctttgaaacccctgaggtaggtaggctctataaggttttccaaactcaatcttgggctatcattgagcgtgccttctccagtttgaagatccgcgtgtccaaagctttatcagcatttataagttttctgcaaaaccttgcagataactttactgcaataagtggtgttgtcactgcactcattcgtgaactccaggatcttaccctggatgtggcgacacgtatcactaacatacaatttgtttaccgtgccggtaagcttattgtcgacacgacaagtgtcatagctaaacttttccagccattttgtgattttatatcacctttccttcggaaagttgctggttttgcaatttacactgttggtaatcgcatgcttatgtttaccagcactggcacctttcttctcacaaaggcaactactaagatactcaataaggcaaagtacatctttgatgtggagcctgagtacccagtagatgtaacaacatccaaagttgtagtacatgaagcactccagcaaaccgacactaagcctactagagctctggaggctgttgatgtcgttgttggtaatactgtactgcaaatggctactgatggcactgcgttctacccatcggatggtacgcacgcctctcttccaggattcaaagcaggttcggatgagcttttcataagcttcagctgcgacctctttgatgatgagactaatgctcaaatcaacgaaacactcgctgcatatgagcttaaccaactagtggctccaggtgattctacaccgcgtcaaattgcgacgttggttgtcgatacacttgcagatgctataacagaccactttccggagaaaaccattgatctacctgaagactatcaagtcttttctgaccatgatgacctcccactcgcacaataccacatccctgatcacctgagcctgtatattcaggctatggaaggtgaagatgatagtggtgatgaaatatgtattgaggacgatgattacgactgtcctcaagccgacgaagacacagaaggagtaattccccaacagtgggaacttcctgatgttgataaatttttactcaagatccaggaacggaagaccagcagcgacgaagtacttagcgtcgacgtctatcctaaaccagagccggtcggcaatgttgggattgacgacagcgcgtcggaaaagaagccaaatggggactcagtaccggatcctgaggtccatccaacactagagagtgtggatgttgaacgaccaaccgaaacagcaaaccaggctgttgaagacaaaccttctgataccacctttgtggttgatgaggaacaattacaagaatcaacaccagaacatgaactccgctcctatgaaggggagtttgattctgatgatgaaattattattcctatagtaccagtaacacctgcggatttaaaaccacagactattactataaaggagtactttaagtctgaaaaacttgagactattaacgaaggatccacagagtcagttacacaatctgacgattcgtttgacgagtcatttgttgatgctgagtctgatgatccacaagatcctgctgtatatgatgatacaacaattataacggacagcactgatgtaggcgatgagcctgagacaactctagctaccatcgttaacacacctctgacactcgataataacttgccacctgaagccattaaacaacccagcccaactaaggttgagttagttgttggtgaattggcgagtattaaatttgacaattctgttctagtcaaccctgctaatgcgcaattaacaaatggcggtggagctgctcgtgcaattgcaaaattagctggtccaaaatatcaagagtactgtaatagtgtggctcctatctcaggaccgcttaccacggactcttttgatgccaagaaatttggtgtagcctgcatcttgcatgtagtgccacccaaaggttctgaccctaatgtacaagaactcctgtatcaagcttacaagagtatccttactgaaccagcacactatgttatacctatactaggtgctggtatctttggatgcaacccagtccactctctggatgcgttcaggaaagcatgtccaagtgacataggtcgtgtcacccttgtcactatgaacaaaaaccatttgcaggtgtgggatgctctcaataggaccattgtacgcaccactactgactatgatcaagttaccaccaaggcccttacaccccagggagtgttagaagccaatctctttgatggtgaggactttgttcaagaaccaaaacccggtcaaatctaccttgaggttactgaagaagttcagaaccaagccaaggaacttgaccttaaccttcagcaatactgcgtctacctgaagacttgccaccataaatgggttgtgagtcgtacgaacgggttgatgcatctaaaacaaaaagataacaattgttttgttagtgcaggtgtaaacctgtttcaaaacactgcttatcaacttagacctgctattgatgctctctatagggagtatcttaatggtaatccaaatagatttgttgcttggatctacgcatccactaaccgtcgtgttggtgagatgggttgtccacagcaagttatttctttgctcgttagtaactctgacgcagcattttcagcaactacagcctgttgtaacacctactttaaccacacaggtgttatttcagtagctcgtgaatatgacccaatacaaccaaaggtctactgcatgaagtgtgatgtgtggactccctttacaccccagagtggaaaaggtgcagttgcaattggtatttctgcagatgaacctaccggtcctgccattaaatttgccgcagctcactgctggtacactaatggcaagaaaacagttaatggctatgacactaaagctaatgttgtagctacctatcataggtttgacgtgcctaagcctcaacttgtcgaggacgtggttgcgctgcctactaaaaatgactttgaagttctcaatgttgaagaactgccgcaggatagtgtgctccatttggacccacctcctgtacaggccttacaacctaaggctaaccaacacattgagattctagaaaacccagattatctggacattttggatctttggattcgtaaacccaaattcatcctcgtaaagtcgtggagtgttttgggtagagcactatgtaaggcaggtaaagttgtctttgtcagtgcttcgcttttgacccgtttctacaattaccttgtagagattggtgctcttgactcaacaataaggttgtcagtcgatcttacctgtaaatttgttagaacggttctcccatcgtctaacactgtacacaaaacttgtcttggtctgtattattcagcccagacactttttgtttctttagcaccattccttatgttaccagctgtagttagtctgcttaattcaggctatacaattggcacatatttgtatgcaaaaactggctggccttgtaattacaatgccacgcaacactttgattataattcttactgtgcaggtgacttggtttgtcaagcctgttttgacggtcaagactccctacatttgtatccgcatttacgtgttaatcagcaaccccttcagaccactgactacactgtttatgcgctttcactaatactactattagctaacatgactcttgtcatgggcacgctaatagttactttctttgtgaacttctatggtgtgcaaataccattttatggtacacttttgatagattatcaatccgcactggtgattactttctcagtgtactacttttataaggtaatgaagtttttccgccatctcacacatggatgtaaaattccaacgtgtgtggtatgtgccaaacttcgtaccccacctactataacagttgagactgtcgttcagggcaggaaatacccatctgttattgaaacaaatggcgggtttacaatttgtaaagaacacaacttctattgcaaggactgctctttacaaacacccggcactttcattccgacagaagctattgagtcgctctcacgagctaccaggcttagtgtcaaaccaacagcaccagcattcttacttgctagagatgttgagtgccaaactgatgttgtcgttgctcgcgcaatgcataaccaaaatgcgcatgtgtgcatttcaaaatactctgatatccgtaccgttgaccaactacttaagcctactccactgttttcatacactcccgatgttatcatcgcggcagactttgacaacagaggtagtcttaagacagctaaagaattagctgtggttttgtcaatggaccttaaacgtactataattatcattgatcaggcctattctagacctattgataattatcaggaagttgcttctcgtattgagaagtattacccagttgcaaagatcacacccacaggtgacatctttacagacattaagcaagcgaccaatggccaagctagtgactctgctattaatgcagctgttctggctgtccagcgcggtcttgattttacaattgacaaccctaacaacatattaccacattacgcctttgacttttcaaccctcaatgcagaagaccagtctaccattttggagagtggttgtgctaaaggcaatctcaagggcactaatgttggtgttgttctttcagctagccttgttacacgtcttagtcagcaggctatacgtgtgattgctaatgctgcttcacgtaatggtgttacatgtgctgttactccttctacacttgttatgcgtgggaatattgcaacacagcccttgactcgcatcaaagctggtgcacctcccatgcgtcaaaaaattttatgtgttatcctggcacttgctattgtgtactttgctgctatggcttttggctttttggcaagtcaaattacgcttaatacagtgcctacgattaaatctgatatccgcgcctctaccttctacgttgttagagatggagtcttggatactgttcgttcaaatgacaagtgctttgcaaataagtttttggcatttgatagcttcattcaagcaccttacactaattcacctgactgtccagttgttgtgggagttgttgatgtaacgacgcactctattcctggaattccagcaggtgtcattcatagagacggtctcatacttaacatttatgaacagtctctttatgaaactcatcagcgtcagtctatggttagggatgcgttgtcactcaagacagcaaatctctttaacctaggcaagcgtgttgtagtaggatacactcaacatgaagttgttgtgggtacctcctattttaattctcctgcactttttaatgcaaagtgcaccttcttacagtatcaggacactagacaactctattgctatgatactgttcctactgaacataagctctactctgatgtgcttccgcacgtcgagtataaggctattgacattaatggtgatcttgttcctttcaagataccagagcagataatgttctatccacatattgtgcgctatactagcaattcctattgccgtatggggcattgttttaatactaaccctggtatttgcatttcatttacggacgaatttccgtatagtgaaaatgtcaaacctggtgtgtactgtgctgatacctctttgcagttgttttcaaacctcgttttgggcactgtatctggtattcacatctttacatcaacagctgcattgcttggatctactattgtgatcatactatgcgttgttgctgttcttgcagttcagcgattcttcaaggagtacacaacttttgttatgtacacttgtggtcttgctcttgtcaacattgtaggcattgcacttatgtacaagtgccttgtcttcgcgattttctattatgcaatctacctttactttgtccttactttcccctcctttaagaggaatgtggcattgttttacttcgctgtagtgatcgtgccgcacgtgagtaacatgcaattgcttgcgctcattgtgtgtagcattatctactttctctacacctatgttcatactgtagctaagacagctgggaaattttcttccttcttagacgcagctaaagctacttttgtcattgacaatgaaaagtacgtgttgcttaaagacctcgctggtgctgaatttgaccagtatctggcctcttacaacaagtacaaatatttttctggtactgcttctgataaggattatgataaggtctgtatggcatttcttgccaaggctttgtcatcttttcgtgaaggaggcggttcacagttgtacacaccacctaaatttgcagttgttcagagtcttaagaccaagctgcaagcaggtatcaaaatcctcctgcacccttcaggtgtagttgagcgatgtatggtctcagttgtctacaatggatctgcattgaatggcatctggcttaagaatgttgtctactgcccacgccatgtaattggaaaattccgtggtgaccagtggactcacatggtctcaattgctgattgccgcgactttatagtcaagtgtccaatacagggtattcagctaaatgtccaatcagttaagatggtaggagctctcctccagttaactgttcataccaacaacacagccactccagactataagtttgaaaggctccaaccaggatcatcgatgacaattgcttgtgcttatgatggcattgtacggcatgtctatcacgtggtcctccaacttaataatcttatttatgcaagcttccttaacggagcttgtggtagtgtgggttacactcttaagggtaaaacactctacttacattacatgcaccacattgagtttaataacaaaactcatagtggtacagatcttgaaggtaacttctatggcccctatgtggatgaggaagttattcagcaacaaacagcattccagtattacactgataatgttgttgctcaattatatgcacacttactgactgttgatgctagaccaaaatggctggcacaatctcagataagtatcgaggatttcaactcatgggctgctaacaattcctttgctaacttcccatgtgaacaaactaatatgtcctacattatgggactctcgcaaacagctcgagtccctgtagaacgtatcctcaataccattatacagctaaccaccaatagagatggtgcttgtattatgggatcttatgatttcgagtgcgattggacgccagagatggtatacaatcaggctccaatttcattgcagtcaggagtagttaagaaaacttgtacgtggttcttccacttcttgtttatggctattaccatgctactcgctgccatgcatgttttccctgtacacttgtatccaatagtactgccatgcttcactgtcgtggcattcctgttgactttaaccattaaacacactgttgtgtttaccactacatacttgcttccgtcacttttgatgatggttgtaaatgctaacactttttggataccgaacacatttctgcgcacctgctacgaaactatattcggttccccaattgctcagcgactgtatggttacactgttgctctttatatgctgatctatgctggacttgcaatcaactatacgttgaaaacactccggtatagagcaacttcattcttatctttttgcatgcagtggtttcaatatggttatgttgcacacattgcgtacaaactgcttaataaaccctggacagaatcactactcttcacagccttcacaatgctaaccagtcatcctttgttggctgctcttagctggtggctagctggtcgcgtaactctgcccattatcatgcctgacttagctattcgtgttttggcgtataacgtcattggctatgtcatatgtgttcgatttggccttatgtggcttgcaaatcggttcacaactgtacctatgggcacataccagtatatggtgtctgtagagcaacttaagtacatgatggcagttaagatgtccccaccgcgtaatgcgtttgaggtgcttatagccaacattagacttcttggtttgggtggaaaccgtaacattgctgtttctactgtccaaaacaaaattcttgatgcaaaagctactgctgttgttgttgctaaccttcttgaaaaggctggcgtcacaaacaagcacgctatttgcaaaaagattgtgaaactccacaatgatacccttaaagccaccacttatgaggaggttgaggtagcacttgtgaaacttctttctcacataattgagttcttgccaactgatcaggtagatgcttatctagctgatgcggccaatgctcaacatgttaatacctattttgacaacttgcttgagaacaaagctgttgttcaggctgttgccgatatcaacattaatctggattcttatagaatttataaggaggcagatgctatttataaacgatctgttgagatgaacgaatctccgcaggagcaaaagaaaaagcttaaagctgttaacattgcaaaggcggaatgggagcgtgaggctgcttctcagcgtaagcttgaaaagcttgctgatgctgctatgaagtctatgtatcttgcagaacgtgctgaggatcgtcgcattaagctaacctctggacttactgcaatgctttaccatatgcttagacgtcttgactcagatagggtaaaagctctgtttgagtgcgctaaggcacaaatcttgccaatacatgctgtagtcggaatttctaatgacaaccttaaagttatttttaacgataaggacagctactctcattatgtagagggcaacacacttatacataagggagttcgctacactattgtgaagaaactctccttagataatgcacctattgaaggcgtaccagaagaattccctgtggtcgttgagactgttagggaaggtgtgccccagttgcaaaataatgagctatgtttgcgcaatgttttcactgctcagaacacagctcaggacttcaatggcaatgaatccactgtaaaatctttttatgttactagaaccggtaagaagattttggttgccattacatcaactaaagacaatcttaagactgtgacctgccttactgagaccggtaagacagtccttaacttggacccccctatgcgcttcgcacataccgtaggtggaaaacagtctgttgtctatctctattttattcagaatattagttcactcaacagaggtatggttattggccacatctctgaaactactatccttcaggcaagtggcactcaaattgagtaccagcaaaatgcctctcttttgacctatttggctttcgctgtagaccctaagacagcctaccttaagcatcttgctgatggtgggtctcctatacagggttgtattcagatgattgctactatgggtcctggatttgcagttactactaaaccacaacctaatgagcatcagtattcttatggtggtgcttcaatttgtctttattgccgtgctcatataccacatcctggtgttgatggacggtgcccctacaaaggccgctttgttcacatcgacaaagataaggaacctgtttccttcgccttgactcatgagccatgcagttcttgtcaacggtgggttaattatgactgcacctgcggatctagtctgcagaattcggcttatttaaacgagtaacgggttctagtgacgcccggctagaacccctgcagcctggaactcaaccagatgctgtaaaaagggccttccatgtgcataatgataccacctctggtatattcttaagcacaaaatctaactgcgctcggtttaaaaccacacgcagtgccctgcctttacctaataagggagaggttgaattgtactttgttactaagcagtgtgcagctaaagtcttcgaaatcgaggaggaatgctacaacgctcttagtacagagctttatactactgatgatacatttggtgtccttgccaaaactgagttctttaagtttgacaagatacctaatgtcaatcgccagtatctgactaaatatacactcctggacttggcttatgctctacgtcatttgtcaacatctaaggatgttattcaagaaatcttgatcaccatgtgcggaacccctgaagattggtttggggaaaattggtttgatccaattgagaacccatccttttacaaggagttccataaacttggggatattcttaaccgttgtgttcttaatgccaataagtttgctagtgcctgtatagacgctggtcttgttggcatattaacacccgacaaccaagacctcctgggtcagatctatgactttggagattttattattacacaaccaggtaatggatgtgtggacttagcatcctattattcttatttaatgcccattatgtccatgactcacatgttaaagtgtgagtgtatggatagtgatggcaacccacttgagtatgatggatttcagtatgacttcacggacttcaagcttggcttgttcgagaagtattttaagtactgggaccgtccttaccatcctaacactgttgaatgtccagatgaccgttgcgtattgcactgtgcgaacttcaatgtgttgtttgctatgtgtatacctaatacggcatttggcaatctttgttcaagagctactgttgatggccaccttgtggtccagacagtgggtgtacacttgaaagaactcggtatagtccttaaccaggacgttaccacacacatggcaaatattaatctaaacactctattgcgattggttggtgatcccaccactattgcaagtgtctcagacaagtgtgtagatttaagaactccttgtcagaccttggctactatgtctagcggaattgctaaacagtcagtcaagcccgggcattttaatcaacacttctacaagcatttgcttgatagtaacctattagaccaacttggaatagacattcgccacttctactatatgcaggatggtgaagcggctatcacagactacagctactacaggtataatacccccacgatggtagatatcaagatgttcttattttgccttgaggtggcagataagtatcttgagccctacgaaggtggatgtattaatgcacagtcagttgtggtctctaatttggacaagtcagcgggctacccctttaacaagctaggtaaggctcgtaactattacgacatgactcatgccgagcaaaatcaactgtttgagtatacaaaacgcaatgttttgcctacactcactcagatgaaccttaagtatgcaatttcagccaaggatcgtgctcgcactgtggcaggagtgtctataattagcaccatgactaacaggcagtaccatcaaaagatgctgaaatctatttcacttgcacgcaatcagaccatcgtgattggaacaaccaaattctatggtggttgggacaacatgttacgacgactgatgtgtaatatcaacaatcccattttagtgggttgggattaccctaagtgtgatcgttctatgccaaacatgctgcgcattgccgcttcgtgcttgctagcacgaaaacacacttgctgtaaccaaagccagcgattctaccgtttggctaatgaatgttgccaagtactatctgaagtggtagtctctggtaacaacctctatgtaaaaccaggtggcactagcagtggtgatgcaaccacagcttatgccaactcggtatttaacatcttacaggtggtttctgctaatgtagccaccttcttatcaacttccaccacgacacatcttaataaggacattgcggacttgcatcgtagtctttatgaagatatttatcgtggtgactctaatgatatcaccgtcatcaatagattctaccagcatctccaaagttactttggacttatgatattgtctgatgatggtgtcgcatgcatagactcagccgttgcaaaggctggagctgttgctgatcttgatggtttccgagacattttgttttaccaaaacaatgtttacatggcagactcaaagtgttggacagaaactgacatgaatgttggccctcatgaattttgctcacagcatactgtgttagcagagcatgatggtaaaccttactacttaccttacccagatgtctctcgcattctgggtgcatgtatctttgtggatgacgttaacaaggctgaccctgttcagaaccttgaacgttacatctcacttgcaattgatgcatatccccttaccaaggttgaccctattaagggtaaagtcttttatttgttactagactacatacgtgttcttgctcaggagttacaggacggtatccttgatgctttccaatcactcactgacatgtcgtatgtaaataactttatgaatgaggccttttatgctcagatgtatgagcaaagtcctacactacaggccagcggtgtttgtgtggtgtgtaattcacccactatactgcgctgtggtgattgcattcgtcgaccactactttgttgcgtctgtgcctaccagcatgttacgcagactacacataaacgtatcattgctatcaacaactacatctgtagtgttgagaattgcaatgaggacaatgttgaaaaacttttcatttctggcactgcgatttattgtgagaatcacaaacccacgctgtgcatacccattgtagctaatggttctgtttttggtatctatcgccacactgcccgtggtagtgatgacatagacctctttaacgagcttgctacatctaactatgacactattgaaccttatcagaaggccaatcgtgcacctttatcacttatgctcttcgctgctgaaaccattaaggcactcgaggagtctatcaagaagtcatatgctaccgcaaccgtcaaggatgtgtatgaccaacgcttcattaaacttctatgggaacagggtaaaaagccgccacccataacgaagaaccacattttcactggctaccattttaacaagaatggaaaaacccaagttggtgattacattcttgctaaaacagatggcagtgacacttatacttacagaggaacatctacctacaaactccaaacaggtgatgttctagtcttaatggcacatgttgttacaccgctctcagcaccccctgtgctaacgcagacaacatatgtcagaaaatcacttttacccgactctgttggtgcgtcttattatgtgcaacattttaagtcatataatgagatagctatgcagagggttacaacagtattaggtccaccaggcacaggtaagtcaacctttgctattggtttggctaagtactttcccagtgcacgtatttgctacactgcgtcttcgcatgcagcaatcgatgcactctgtgaaaaagctttcaagacaatacctgtaggccaatgcagtcgtatcgtacccacacgtacaactgttgagtgctttcaggagtttgtcgtaaataacacaactgcacagtatatcttctcgactatcaatgccttacctgacattaagtgtgacattgtagttgtagatgaggtttctatgttgaccaattatgagctttcctctgtgaatgctcgtttggtttacaatcacattgtgtatgttggtgatccttatcagttaccttcacctagaactatgcttacgtctggccagctttcgccagctgactataacgtagttactgatataatggtacatgcaggagcggatgttatgctcgacatgtgctacagatgcccacgtgaaatcgttgagacagtgtctaaacttgtctacgataacaaactaaaagcggcgaaaccgaactcaagacagtgttacaagaccattgtgaactttggtcctggagacgttgctcatgagggacaatctgcctacaacgaagcacagttgcgtttcgcactcgcatttagacaacaaaagcggtgggataacgtgactttcatatctccatataatgctatgaatgtgaaagcatccttagcaggtttctctactcagaccgttgactcttctcaaggttctgagtatgattatgttatcttttgcgtgaccactgattcagcacacgcacttaacatggctcgtttgaacgttgcccttacacgcgcaaagataggtatccttgtggtgtttaggcaggcaaacgaactttacaatagtttgcagtttgaatctattgattcacagcttcagtcgagtgctgagaaaaacctcacaccactgtttaagcgctgcggctatgagtataatggcgtccatccagctcatgctttgacctggcatgattgtggtgcagagtaccgctgtgaggagccacttgctaaattagtaggagttgccgatggcactcttatatcatacaaaaccctagtatccacacttgggtttcttccatcacttaaaattgatgcatatcataatatgttcctaacacgtgacgcgtgtcgcacctatgttcagagttggatcggcatagatgttgaagcagcacacgccataaaacctaacaccgggactaacctgccattgcaaataggttttagtaccggaaagaatttttcagtcactccagagggaatttgggtaaacgagcacggatcttgcactgagcccgtccctgccaaaatacctcctggagaacaatttcgtcaccttaaaaaggacatgcgccaggcgcgtccttggaaggttgttcgacgtgagattgctactcacattgctgaggtagctcctcacactgattatatatgctttgtcacttgggctcaccagcttgagctagcgacaatgcgctactttgtcaaactaggtatggaagagaaatgcttttgtggcaggcgggcttgtttcactaatggaactgagttcgcttgcaaagcacaccattctctcaccattccacaatgtgattatgtgtacaatccattcctcatcgacgtggctacgtggggattctcgggacggctttccaccaaccatgacgctgtgtgcacatatcatgctaatgcccatgttgcatcagctgatgcaatcatgacggtatgtttagctatccatgaactgttcagtactgttgactggaactttgaatttccagtaactgctgagcaatcgcaacttaacaaggcctgtcgcttagtacaggcgaattacttaaatatactactcactacaaccaaagccacggtggttcacgatattggtaacccaaaaggtatccctatcgtgcgcaaacctggtgttaaatatcacttctatgatcaagcacccattgtcaaacacgttcaaaaactaaagtacaagccagagatggaggcccgtttcaccgatggtttgactatgttttggaattgtaatgttgacacataccctgctaacgcccttgtgtgccgctacgacactcatcggcagaagcatttaattggacctaatggttcagcactatatgttaataagcatgcttttctcacccctgagatgcatacttatgctacacataaactcaacttggctccactcatctactactccaccacagattgtagtagtgaacagcctattgttgttacctacagagattgtgtcacccggtgcaatactggaaaaactctctgtccaaatcatgctcttgaataccaagagtttatcaatgcatacaatctcatggctcgccatggatttaatgtttacataccacgcaatgtcaacgtttacaactgttggcttactttcactaatctccaaaaccttgaaaacttagcttacaactgttattataagaactgcaatgctcacgttgatgggcagcttgatgtagttattaataataacgctgtatatgctaaggtcgacaataatcttgtcaaacttttcgacaaccgcactaacttacctgtctcagtggcctttgaacattacactaacaggcatacccgttcactgccaactacacagctgttatctggtttaggcgtaaccgccaccagaaatttcactgtgtggttcgacaatgatacaattttccaatacactattaatgtatctacgtatactgacatcgaccctagtacccatgttgtcctctgtgatgataggtacggaacagattggagtcagtttaaccaacttcctaatgcagtattcctcaccaaaactaaggtgaagaaaacagaaccgtttgtttgtacagcactgaccctaaatggcgtcgccattgacggtgaagagctatacatctatgtacgctataacaatcaactgaccacatttgctactacttgtacacagggtagaaatgttgagcagtttatacctaaaacacctatggaaagagacttccttgagatgtctcaacagtccttcatccagcaacatcaattgcaggaactgggtgttgaacacattatctatggtgatgattccagtccagtcattggcggaactcacacacttatctcactagttaaaaacaagtttgaacatcagcttgtcaaccatgtttacaacccagtccagaactgtgttgttacctcacctaacgcaagctccaagaacgtttgcactgttcttgatgttcttcttgatgactacattgacatcataagacaagcacatgccagttacacaagtaaatccaaagtattcactgtgtcaattgacaaccaacaaattagattcatgctttggcatgatgagcaagttaagacttgctacccaatcttacagtcacttaccaatggttaccagatgccatctgtgtacaaaacattggttactgacttacaacctgctgacatccctaattatcattcctacaccccccgggtgcctggagtagttaagaatgttatcaagtaccgccaacttttcaactacatagttaaaaaggataggttggcagtaccacacaatatgaccgtattacaccttggagctgcatctgcactaggtacagcaccaggttcttcagtcataaaacaaatgtttcctgaaggaactgttcttattgacctcgatataagagagttcacttcagatgctaaccaaataatagttacagactacagaacttacataccaccacaccacgtagacgtcatattttctgacctctactgttgtgatgacatacacttctttgacaatctaataaggatagttaaggagaggctcgccctcggtggttctatctttgttaagataactgaacattcattctcacccgaactctactcacttgcgggttggttcgatgattatcaactattttgcacagcagtcaatgcctcgtcttcagaagcatttttatgctgttttaattatttggggcttgctaaggaaaacattaatggttttaacttacatgcttcctatattcaatggcgcaatgaaatagcgttgacaccaacctattctcctttagcggacaacccggctacggcctgtaagctaaaagcaacgcctattatctcggctcgtgagttagagaagaagcctattcttcgctatctcgttgcatcagggcgccttcttgtgaggccaccagaatgcagagagctctattgattatgaccttactttgtctcgttcgagcaaagtttgctgatgatctactcgatttgctcaccttcccgggtgcacatcgcttcttacataaacccacgaggaattccagcagtctctactcgcgggctaataataattttgatgttggcgttcttcctggctaccccactaagaacgttaacctcttctcaccacttactaactccactttgcccattaatggccttcatcggagttaccaaccactcatgctgaattgtcttactaaaataactaaccacactctcagcatgtatctcctacctagtgagatacaaacttatagctgcggcggtgccatggttaaataccagacacatgatgcagttcgtatcattttagacctcactgccactgaccacatctctgttgaagtcgttggccaacatggtgaaaattatgtgtttgtttgcagtgagcagtttaactacaccactgcattacacaaatctaccttcttctcacttaattctgagctttattgctttactaataacaactacttaggtattcttccacctgatttaactgactttacggtctaccgtactggtcagttctatgctaatggttaccttttaggtactttacctattacggttaactatgttaggttgtatcggggtcatttgtctgccaatagtgcccactttgcccttgcaaacctaaccgatacactcataacacttaccaatactactatatcgcaaatcacttattgtgataagtcagtagttgattcaatagcatgccagcgctcttctcacgaagtggaggatgggttttactccgaccctaaatctgccgttagagctaggcaacgtactattgttacactacctaagctccctgagcttgaagtagtgcagttaaatatttctgcacacatggattttggcgaagccagacttgacagcgttaccatcaatggtaacacatcctattgtgtcaccaagccttactttaggcttgaaactaactttatgtgtacaggttgcactatgaatctgcgcactgatacctgtagttttgacctgtcagcagtaaacaatggcatgtcattctctcaattctgtctaagcactgaatctggtgcttgtgagatgaaaattattgttacctacgtatggaattacttgctaaggcagcgtttgtatgttactgctgtagagggccagactcacactggaaccacttcagtacatgcaacagacacttctagtgtaatcactgatgtctgcactgactacactatctatggagtctctggtactggcattattaagccatcagatctcttattgcacaatggcatagcattcacctctccaacaggtgagctttatgcatttaaaaatataaccactggcaaaacccttcaggtcttaccgtgtgaaaccccttctcaactgattgtgataaacaacaccgttgtcggtgctatcacatccagtaattcaactgaaaataataggtttactactactattgtcacacctactttcttttattccacaaatgccaccactttcaactgcactaagcctgttttgtcctatggacctatcagcgtgtgtagtgatggtgcaattgtgggaacatccacattacagaatactcgaccatccatagtttcactatacgatggcgaagttgaaataccatctgcattttctctttccgttcagacggagtacttgcaagttcaagcagagcaagttatagttgattgtcctcagtatgtatgcaatggcaacagccgttgtctacaattactggcacaatacacctcagcttgctctaacattgaagcagctctgcattcctctgcacagttggatagcagagagattataaatatgtttcaaacatcaacacagtccttgcagttagctaatattaccaacttcaagggtgactacaattttagcagcatactaaccaccagaattggtggcagatctgctattgaagaccttctttttaataaagttgttactagtggccttggcactgttgatcaggactacaaatcctgctctagagacatggccatcgctgacttagtttgttcccagtattacaatggcatcatggttctacctggtgttgttgatgctgagaaaatggcaatgtatactggctctcttactggagctatggtatttggaggactgactgccgcagcggcaataccatttgccacggcagtacaagctcgcctcaattatgtcgcactgcaaacaaatgtactacaagaaaaccagaaaattcttgcagaatcatttaaccaagcagttggcaatatatcacttgcactatcttctgttaatgatgccatccagcaaacttctgaggctcttaacaccgtagctattgctattaaaaagattcaaacagttgttaaccagcagggcgaggcattatcacacctgactgcacagctgtcaaacaattttcaagcaatttcgacttctattcaagacatttacatccgtcttgaggaagtagaggctaaccagcaagttgaccgtctcatcacaggacggttggctgcacttaatgcatatgttactcagttactcaatcagatgtctcagattagacaatctcgattgttagctcagcaaaagattaatgagtgtgtcaaatcacagtcgtccagatacggtttctgtggaaatggcacacacatcttctcacttacacagactgcaccaaatggcatatttttcatgcatgcagtgctagtacccaacaaattcacacgtgtcaacgcttctgccggcatttgtgtggataatacgagaggctactcattgcagcctcaacttatactctaccagtttaataactcctggagagttacacctagaaatatgtatgaacccagactgccccggcaggctgatttcatacaattaactgattgcagcgttactttttacaacaccaccgctgctaatcttcccaatattatccctgacattatagatgtcaatcaaacagtcagtgatattattgacaatttacctacagcaacacctcctcagtgggatgttggtatctataacaacactattctcaacctcaccgttgagattaatgatctacaagagcggtctaaaaacctctcacagattgcagatcgtttacaaaattatattgacaatcttaacaatactctagttgaccttgaatggctcaacagagtggaaacttaccttaaatggccgtggtatatatggcttgccattgccctggctcttattgcatttgtgacaatcctcataacaatctttctttgtactggttgttgtggtggttgctttggttgttgtggcggttgttttggccttttctctaagaagaaaaggtataccgacgaccaaccaacaccgtcctttaagtttaaggaatggtagtcgacgactgggccgttaccatccctggacaatatattattgctatactagttgtcatctgcattggtgtggcactactttttattaatacttgcttagcttgtgttaaattattttacaagtgctacctaggggcagcatatcttgttaggcctattatagtgtactactccaagccgaaccccgtacctgaggatgagtttgtaaaagtacaccaatttcctagaaacactcactatgtctgacgcagaagagtggcaaattattgttttcattgcgatcatatgggcgcttggcgtcatcctccagggaggctatgccacgcgtaatcgtgtgatctatgttattaaacttattctgctttggctgctccaacccttcaccctagtggtgaccatttggaccgcagtcgacagatcatctaagaaggacgcagttttcattgtgtccataatttttgccgtactgaccttcatatcctgggccaagtactggtatgactcaattcgtttattaatgaaaaccagatctgcatgggcactctcacctgagagtagactccttgcagggattatggatccaatgggttcatggaggtgcattcccatcgaccacatggctccaattctcacaccagtcgttaagcatggcaagctcaagctacacgggcaagagctggccaatggcatatcagtcagaaatccgccacaggatatggtgatagtgtcaccaagtgacacctttcactacacttttaagaaacctgtggaatcaaacaacgatccagaatttgctgttctgatataccagggtgaccgcgcttcaaacgctggacttcacaccataaccacttcaaaggccggtgacgctcgcctgtataagtatatgtaatgtgcaactgccatctgcagctgcgagatttatatagattgtgcaataagcggcacatcagaagagaggatgttcctgagcttattgaccctctcgttaaaactcgctgttttgcttacagtctcgtggttcttgctaatgctaatccaattgcatttagcatactacctcggaaaattcttatcaatggtgagcctttactgcttgaatatggtagcatatatggtaaagactttatcattcgaccatcgctccaagtcattcttgaagatgaattaaattaaagttttgacaccaatctatcatggctgcaccagtagtccctactactgacgcgtcttggtttcaggtgctcaaagctcaaaacaaaaaggctactcatcctcagtttcgtggcaatggagttccgcttaactccgccatcaaacccgttgaaaaccatggctactggctgcgttacaccagacaaaagccaggtggtactcccattcctccatcctatgccttttattatactggcacaggtcccagaggaaatcttaagtatggtgaactccctcctaatgataccccagcaaccactcgtgttacttgggttaagggttcgggagctgacacttctattaaacctcatgttgccaaacgcaaccccaacaatcctaaacatcagctgttacctctccgattcccaaccggagatggcccagctcaaggtttcagagttgaccccttcaacgctagaggaagacctcaggagcgtggaagtggcccaagatctcaatctgttaactccagaggcacaggcaatcagcccaggaaacgcgaccaatctgcaccagctgcggtacgtcgtaagacccagcatcaagctcccaagcggactttacccaagggtaaaaccatttctcaggtatttggcaaccggtctcgtactggtgccaatgtcggctctgcagacactgagaagacgggtatggctgatcctcgcatcatggctctagccagacatgtgcctggtgttcaggaaatgcttttcgctggccaccttgagagcaactttcaggcgggggcaattacccttaccttctcctactcaatcacagtcaaggagggttctcctgactatgagagacttaaggatgcgctcaatacggtcgttaaccagacctatgagccacctaccaaaccaactaaggacaagaagcctgacaaacaagaccagtctgctaaacccaaacagcagaagaaacctaaaaaggtaactctgccagcagacaaacaggattgggagtgggatgatgcttttgagataaagcaggaatcagcagcgtagacatcaatctatgtctgttaaacccacccaactccactcaaatatctctttggttccagagagtcgtagtgtatagccagagagccagtcagagggcgctatcatgcaaactagggctggctactctagcacagaatcacatcccgataatcaacagtgctagaaggttgattataccatttaatatgccgaggccacgcggagtacgatcgagggtacagcataatctcaacttttgttgagccacaattttaatcctaattggagaaagccaaaggactgtactacttttgtgggtgtagcagtcgcccagtgggaaagcgccaactaggttacaattgtggtggggacaaattaggggaaattaaattggcttataggggggatggagcgg

SEQ ID NO: 12 - DNA, 25402 bp, Artificial Sequence
  full length encoding DNA for PDCoV virus NVSL USA/Michigan/8977/2014
ttatagcattagtctataattttatctccctagcttcgctagttctctaccgacaccaatccaggtgcgtctgccaccaagttggctacccttcctaggggcgctttcgcgcttgctcaccattagattacctggaaaccagccattcaggttggagtttccccaggctcttttgtgtgggcattagcggcttgtggtttttgcacaaaatctaagctacttaccgttcctctgaccatccaccacttctatagacagcactgattaccgtagggtttaagtcacaccggtctgcaccgcccgtcagcggacacattacccagcatagcactccttgcaccgagcctaggtaggataaaaccccctaccgggtgactcttaaggcgtttcctccacgggatagccactagtcactaggtgtaagtgatctgatctgggcgtattgtgttgcgcaagtgtgatacccataggagcgtggaatcctattctgcggctcagtgcctgatatagctgtgaaatggccaagaacaagtccaagcgcgacgctattgcgttgcctgaaaatgtaccaccacctctgcaacttttcattcatgttgcagctgctgaagagggtcaccctaaggttactacttaccttggcaactataacctctatgccaccaaggctccgcctggcgtgcaggttcttagtgctaaaacctctcttactgactttgagaatgtctttggagctcaacccaccttgcgatcaattcgtaatctggtttgtgaggctcgctcggctgaatggacaacttccaagaatgcttttgcactcaaagccactcaacttgactactctgatgccgttttgagggcaatgattcgtttctgccctccaaaggtgtccacactcgttgcctttgctctttttggccgattggttaaaattgaggacaaggaacttgctgagttagctcgtgatactgcccttgagttggcgtacacggctaaaattggtacatctcttgctgacacgagatctgtctcacttattcataaggacgcttatctaactctcagtaatgaggttgttggcgtaacttttactgccgcacttatggcaaaggctaccactgttaatggagcaatgcaatactcaaacttttacctctaccctcgtgccactattaaggtaaccgatggtaaggctgaagcaattgcaactaagcctctgtctgctgccactaaaggcaagcaaatcacagaggatgtcaaccttctccctgactatcagcagctgcttgttgatcaagtgactggcactgaggttaaggttggagctctaacctatgttaagaccactgattcgccacccctttactttcccaaagtcaagggtggtgttattggtattgcacttaagcagcagggcactgcggctaagaagctcaatgtagtcttccatgctcaacctgatgatgttctgctagccttcatacaacttcagcaattcttgaaccgtacttcggattcaagtgttgaaattactgattgccagagttatgaagtatctccaactgtgacggtcaaaattggcccgtctaaacctggggatgtcatcgtggctactgatgaggaataccttaaatgctttgaaacccctgaggtaggtaggctctataaggttttccaaactcaatcttgggctatcattgagcgtgccttctccagtttgaagatccgcgtgtccaaagctttatcagcatttataagttttctgcaaaaccttgcagataactttactgcaataagtggtgttgtcactgcactcattcgtgaactccaggatcttaccctggatgtggcgacacgtatcactaacatacaatttgtttaccgtgccggtaagcttattgtcgacacgacaagtgtcatagctaaacttttccagccattttgtgattttatatcacctttccttcggaaagttgctggttttgcaatttacactgttggtaatcgcatgcttatgtttaccagcactggcacctttcttctcacaaaggcaactactaagatactcaataaggcaaagtacatctttgatgtggagcctgagtacccagtagatgtaacaacatccaaagttgtagtacatgaagcactccagcaaaccgacactaagcctactagagctctggaggctgttgatgtcgttgttggtaatactgtactgcaaatggctactgatggcactgcgttctacccatcggatggtacgcacgcctctcttccaggattcaaagcaggttcggatgagcttttcataagcttcaactgcgacctctttgatgatgagactaatgctcaaatcaacgaaatactcgctgcatatgagcttaaccaactagtggctccaggtgattctacaccgcgtcaaattgcgacgttggttgtcgatacacttgcagatgctataacagaccactttccggagaaaaccattgatctacctgaagactatcaagtcttttctgaccatgatgacctcccactcgcacaataccacatccctgatcacctgagcctgtatattcaggctatggaaggtgaagatgatagtggtgatgaaatatgtattgaggacgatgattacgactgtcctcaagccgacgaagacacagaaggagtaattccccaacagtgggaacttcctgatgttgataaatttttactcaagatccaggaacggaagaccagcagcgacgaagtacttagcgtcgacgtctatcctaaaccagagccggtcggcaatgttgggattgacgacagcgcgtcggaaaagaagccaaatggggactcagtaccggatcctgaggtccatccaacactagagagtgtggatgttgaacgaccaaccgaaacagcaaaccaggctgttgaagacaaaccttctgataccacctttgtggttgatgaggaacaattacaagaatcaacaccagaacatgaactccgctcctatgaaggggagtttgattctgatgatgaaattattattcctatagtaccagtaacacctgcggatttaaaaccacagactattactataaaggagtactttaagtctgaaaaacttgagactattaacgaaggatccacagagtcagttacacaatctgacgattcgtttgacgagtcatttgttgatgctgagtctgatgatccacaagatcctgctgtatatgatgatacaacaattataacggacagcactgatgtaggcgatgagcctgagacaactctagctaccatcgttaacacacctctgacactcgataataacttgccacctgaagccattaaacaacccagcccaactaaggttgagttagttgttggtgaattggcgagtattaaatttgacaattctgttctagtcaaccctgctaatgcgcaattaacaaatggcggtggagctgctcgtgcaattgcaaaattagctggtccaaaatatcaagagtactgtaatagtgtggctcctatctcaggaccgcttaccacggactcttttgatgccaagaaatttggtgtagcctgcatcttgcatgtagtgccacccaaaggttctgaccctaatgtacaagaactcctgtatcaagcttacaagagtatccttactgaaccagcacactatgttatacctatactaggtgctggtatctttggatgcaacccagtccactctctggatgcgttcaggaaagcatgtccaagtgacataggtcgtgtcacccttgtcactatgaacaaaaaccatttgcaggtgtgggatgctctcaataggaccattgtacgcaccactactgactatgatcaagttaccaccaaggcccttacaccccagggagtgttagaagccaatctctttgatggtgaggactttgttcaagaaccaaaacccggtcaaatctaccttgaggttactgaagaagttcagaaccaagccaaggaacttgaccttaaccttcagcaatactgcgtctacctgaagacttgccaccataaatgggttgtgagtcgtacgaacgggttgatgcatctaaaacaaaaagataacaattgttttgttagtgcaggtgtaaacctgtttcaaaacactgcttatcaacttagacctgctattgatgctctctatagggagtatcttaatggtaatccaaatagatttgttgcttggatctacgcatccactaaccgtcgtgttggtgagatgggttgtccacagcaagttatttctttgctcgttagtaactctgacgcagcattttcagcaactacagcctgttgtaacacctactttaaccacacaggtgttatttcagtagctcgtgaatatgacccaatacaaccaaaggtctactgcatgaagtgtgatgtgtggactccctttacaccccagagtggaaaaggtgcagttgcaattggtatttctgcagatgaacctaccggtcctgccattaaatttgccgcagctcactgctggtacactaatggcaagaaaacagttaatggctatgacactaaagctaatgttgtagctacctatcataggtttgacgtgcctaagcctcaacttgtcgaggacgtggttgcgctgcctactaaaaatgactttgaagttctcaatgttgaagaactgccgcaggatagtgtgctccatttggacccacctcctgtacaggccttacaacctaaggctaaccaacacattgagattctagaaaacccagattatctggacattttggatctttggattcgtaaacccaaattcatcctcgtaaagtcgtggagtgttttgggtagagcactatgtaaggcaggtaaagttgtctttgtcaatgcttcgcttttgacccgtttctacaattaccttgtagagattggtgctcttgactcaacaataaggttgtcagtcgatcttacctgtaaatttgttagaacggttctcccatcgtctaacactgtacacaaaacttgtcttggtctgtattattcagcccagacactttttgtttctttagcaccattccttatgttaccagctgtagttagtctgcttaattcaggctatacaattggcacatatttgtatgcaaaaactggctggccttgtaattacaatgccacgcaacactttgattataattcttactgtgcaggtgacttggtttgtcaagcctgttttgacggtcaagactccctacatttgtatccgcatttacgtgttaatcagcagccccttcagaccactgactacactgtttatgcgctttcactaatactactattagctaacatgactcttgtcatgggcacgctaatagttactttctttgtgaacttctatggtgtgcaaataccattttatggtacacttttgatagattatcaatccgcactggtgattactttctcagtgtactacttttataaggtaatgaagtttttccgccatctcacacatggatgtaaaattccaacgtgtgtggtatgtgccaaacttcgtaccccacctactataacagttgagactgtcgttcagggcaggaaatacccatctgttattgaaacaaatggcgggtttacaatttgtaaagaacacaacttctattgcaaggactgctctttacaaacacccggcactttcatcccgacagaagctattgagtcgctctcacgagctaccaggcttagtgtcaaaccaacagcaccagcattcttacttgctagagatgttgagtgccaaactgatgttgtcgttgctcgcgcaatgcataaccaaaatgcgcatgtgtgcatttcaaaatactctgatatccgtaccgttgaccaactacttaagcctactccactgttttcatacactcccgatgttatcatcgcggcagactttgacaacagaggtagtcttaagacagctaaagaattagctgtggttttgtcaatggaccttaaacgtactataattatcattgatcaggcctattctagacctattgataattatcaggaagttgcttctcgtattgagaagtattacccagttgcaaagatcacacccacaggtgacatctttacagacattaagcaagcgaccaatggccaagctagtgactctgctattaatgcagctgttctggctgtccagcgcggtcttgattttacaattgacaaccctaacaacatattaccacattacgcctttgacttttcaaccctcaatgcagaagaccagtctaccattttggagagtggttgtgctaaaggcaatctcaagggcactaatgttggtgttgttctttcagctagccttgttacacgtcttagtcagcaggctatacgtgtgattgctaatgctgcttcacgtaatggtgttacatgcgctgttactccttctacacttgttatgcgtgggaatattgcaacacagcccttgactcgcatcaaagctggtgcacctcccatgcgtcaaaaaattttatgtgttatcctggcacttgctattgtgtactttgctgctatggcttttggctttttggcaagtcaacttacgcttaatacagtgcctacgattaaatctgatatccgcgcctctaccttctacgttgttagagatggagtcttggatactgttcgttcaaatgacaagtgctttgcaaataagtttttggcatttgatagcttcattcaagcaccttacactaattcacctgactgtccagttgttgtgggagttgttgatgtaacgacgcactctattcctggaattccagcaggtgtcattcatagagacggtctcatacttaacatttatgaacagtctctttatgaaactcatcagcgtcagtctatggttagggatgcgttgtcactcaagacagcaaatctctttaacctaggcaagcgtgttgtagtaggatacactcaacatgaagttgttgtgggtacctcctattttaattctcctgcactttttaatgcaaagtgcaccttcttacagtatcaggacactagacaactctattgctatgatactgttcctactgaacataagctctactctgatgtgcttccgcacgtcgagtataaggctattgacattaatggtgatcttgttcctttcaagataccagagcagataatgttctatccacatattgtgcgctatactagcaattcctattgccgtatggggcattgttttaatactaaccctggtatttgcatttcatttacggacgaatttccgtatagtgaaaatgtcaaacctggtgtgtactgtgctgatacctctttgcagttgttttcaaacctcgttttgggcactgtatctggtattcacatctttacatcaacagctgcattgcttggatctactattgtgatcatactatgcgttgttgctgttcttgcagttcagcgattcttcaaggagtacacaacttttgttatgtacacttgtggtcttgctcttgtcaacattgtaggcattgcacttatgtacaagtgccttgtcttcgcgattttctattatgcaatctacctttactttgtccttactttcccctcctttaagaggaatgtggcattgttttacttcgctgtagtgatcgtgccgcacgtgagtaacatgcaattgcttgcgctcattgtgtgtagcattatctactttctctacacctatgttcatactgtagctaagacagctgggaaattttcttccttcttagacgcagctaaagctacttttgtcattgacaatgaaaagtacgtgttgcttaaagacctcgctggtgctgaatttgaccagtatctggcctcttacaacaagtacaaatatttttctggtactgcttctgataaggattatgataaggtctgtatggcatttcttgccaaggctttgtcatcttttcgtgaaggaggcggttcacagttgtacacaccacctaaatttgcagttgttcagagtcttaagaccaagctgcaagcaggtatcaaaatcctcctgcacccttcaggtgtagttgagcgatgtatggtctcagttgtctacaatggatctgcattgaatggcatctggcttaagaatgttgtctactgcccacgccatgtaattggaaaattccgtggtgaccagtggactcacatggtctcaattgctgattgccgcgactttatagtcaagtgtccaatacagggtattcagctaaatgtccaatcagttaagatggtaggagctctcctccagttaactgttcataccaacaacacagccactccagactataagtttgaaaggctacaaccaggatcatcgatgacaattgcttgtgcttatgatggcattgtacggcatgtctatcacgtggtcctccaacttaataatcttatttatgcaagcttccttaacggagcttgtggtagtgtgggttacactcttaagggtaaaacactctacttacattacatgcaccacattgagtttaacaacaaaactcatagtggtacagatcttgaaggtaacttctatggcccctatgtggatgaggaagttattcagcaacaaacagcattccagtattacactgataatgttgttgctcaattatatgcacacttactgactgttgatgctagaccaaaatggctggcacaatctcagataagtatcgaggatttcaactcatgggctgctaacaattcctttgctaacttcccatgtgaacaaactaatatgtcctacattatgggactctcgcaaacagctcgagtccctgtagaacgtatcctcaataccattatacagctaaccaccaatagagatggtgcttgtattatgggatcttatgatttcgagtgcgattggacgccagagatggtatacaatcaggctccaatttcattgcagtcaggagtagttaagaaaacttgtacgtggttcttccacttcttgtttatggctattaccatgctactcgctgccatgcatgttttccctgtacacttgtatccaatagtactgccatgcttcactgtcgtggcattcctgttgactttaaccattaaacacactgttgtgtttaccactacatacttgcttccgtcacttttgatgatggttgtaaatgctaacactttttggataccgaacacatttctgcgcacctgctacgaaactatattcggttccccaattgctcagcgactgtatggttacactgttgctctttatatgctgatctatgctggacttgcaatcaactatacgttgaaaacactccggtatagagcaacttcattcttatctttttgcatgcagtggtttcaatatggttatgttgcacacattgcgtacaaactgcttaataaaccctggacagaatcactactcttcacagccttcacaatgctaaccagtcatcccttgttggctgctcttagctggtggctagctggtcgcgtaactctgcccattatcatgcctgacttagctattcgtgttttggcgtataacgtcattggctatgtcatatgtgttcgatttggccttatgtggcttgcaaatcggttcacaactgtacctatgggcacataccagtatatggtgtctgtagagcaacttaagtacatgatggcagttaagatgtccccaccgcgtaatgcgtttgaggtgcttatagccaacattagacttcttggtttgggtggaaaccgtaacattgctgtttctactgtccaaaacaaaattcttgatgcaaaagctactgctgttgttgttgctaaccttcttgaaaaggctggcgtcacaaacaagcacgctatttgcaaaaagattgtgaaactccacaatgatacccttaaagccaccacttatgaggaggttgaggtagcacttgtgaaacttctttctcacataattgagttcttgccaactgatcaggtagatgcttatctagctgatgcggccaatgctcaacatgttaatacctatttagacaacttgcttgagaacaaagctgttgttcaggctgttgccgatatcaacattaatctggattcttatagaatttataaggaggcagatgctatttataaacgatctgttgagatgaacgaatctccgcaggagcaaaagaaaaagcttaaagctgttaacattgcaaaggcggaatgggagcgtgaggctgcttctcagcgtaagcttgaaaagcttgctgatgctgctatgaagtctatgtatcttgcagaacgtgctgaggatcgtcgcattaagctaacctctggacttactgcaatgctttaccatatgcttagacgtcttgactcagatagggtaaaagctctgtttgagtgcgctaaggcacaaatcttgccaatacatgctgtagtcggaatttctaatgacaaccttaaagttatttttaacgataaggacagctactctcattatgtagagggcaacacacttatacataagggagttcgctacactattgtgaagaaactctccttagataatgcacctattgaaggcgtaccagaagaattccctgtggtcgttgagactgttagggaaggtgtgccccagttgcaaaataatgagctatgtttgcgcaatgttttcactgctcagaacacagctcaggacttcaatggcaatgaatccactgtaaaatctttttatgttactagaaccggtaagaagattttggttgccattacatcaactaaagacaatcttaagactgtgacctgccttactgagaccggtaagacagtccttaacttggacccccctatgcgcttcacacataccgtaggtggaaaacagtctgttgtctatctctattttattcagaatattagttcactcaacagaggtatggttattggccacatctctgaaactactatccttcaggcaagtggcactcaaattgagtaccagcaaaatgcctctcttttgacctatttggctttcgctgtagaccctaagacagcctaccttaagcatcttgctgatggtgggtctcctatacagggttgtattcagatgattgctactatgggtcctggatttgcagttactactaaaccacaacctaatgagcatcagtattcttatggtggtgcttcaatttgtctttattgccgtgctcatataccacatcctggtgttgatggacggtgcccctacaaaggccgctttgttcacatcgacaaagataaggaacctgtttccttcgctttgactcatgagccatgcagttcttgtcaacggtgggttaattatgactgcacctgcggatctagtctgcagaattcggcttatttaaacgcgtaacgggttctagtgacgcccggctagaacccctgcagcctggaactcaaccagatgctgtaaaaagggccttccatgtgcataatgataccacctctggtatattcttaagcacaaaatctaactgcgctcggtttaaaaccacacgcagtgccctgcctttacctaataagggagaggttgaattgtactttgttactaagcagtgtgcagctaaagtcttcgaaatcgaggaggaatgctacaacgctcttagtacagagctttatactactgatgatacatttggtgtccttgccaaaactgagttctttaagtttgacaagatacctaatgtcaatcgccagtatctgactaaatatacactcctggacttggcttatgctctacgtcatttgtcaacatctaaggatgttattcaagaaatcttgatcaccatgtgcggaacccctgaagattggtttggggaaaattggtttgatccaattgagaacccatccttttacaaggagttccataaacttggggatattcttaaccgttgtgttcttaatgccaataagtttgctagtgcctgtatagacgctggtcttgttggcatattaacacccgacaaccaagacctcctgggtcagatctatgactttggagattttattattacacaaccaggtaatggatgtgtggacttagcatcctattattcttatttaatgcccattatgtccatgactcacatgttaaagtgtgagtgtatggatagtgatggcaacccacttgagtatgatggatttcagtatgacttcacggacttcaagcttggcttgttcgagaagtattttaagtactgggaccgtccttatcatcctaacactgttgaatgtccagatgaccgttgcgtattgcactgtgcgaacttcaatgtgttgtttgctatgtgtatacctaatacggcatttggcaatctttgttcaagagctactgttgatggccaccttgtggtccagacagtgggtgtacacttgaaagaactcggtatagtccttaaccaggacgttaccacacacatggcaaatattaatctaaacactctattgcgattggttggtgatcccaccactattgcaagtgtctcagacaagtgtgtagatttaagaactccttgtcagaccttggctactatgtctagcggaattgctaaacagtcagtcaagcccgggcattttaatcaacacttctacaagcatttgcttgatagtaacctattagaccaacttggaatagacattcgccacttctactatatgcaggatggtgaagcggctatcacagactacagctactacaggtataatacccccacgatggtagatatcaagatgttcttattttgccttgaggtggcagataagtatcttgagccctacgaaggtggatgtattaatgcacagtcagttgtggtctctaatttggacaagtcagcgggctacccctttaacaagctaggtaaggctcgtaactattacgacatgactcatgccgagcaaaatcaactgtttgagtatacaaaacgcaatgttttgcctacactcactcagatgaaccttaagtatgcaatttcagccaaggatcgtgctcgcactgtggcaggagtgtctataattagcaccatgactaacaggcagtaccatcaaaagatgctgaaatctatttcacttgcacgcaatcagaccatcgtgattggaacaaccaaattctatggtggttgggacaacatgttacgacgactgatgtgtaatatcaacaatcccattttagtgggttgggattaccctaagtgtgatcgttctatgccaaacatgctgcgcattgccgcttcgtgcttgctagcacgaaaacacacttgctgtaaccaaagccagcgattctaccgtttggctaatgaatgttgccaagtactatctgaagtggtagtctctggtaacaacctctatgtaaaaccaggtggcactagcagtggtgatgcaaccacagcttatgccaactcggtatttaacatcttacaggtggtttctgctaatgtagccaccttcttatcaacttccaccacgacacatcttaataaggacattgcggacttgcatcgtagtctttatgaagatatttatcgtggtgactctaatgatatcaccgtcatcaatagattctaccagcatctccaaagttactttggacttatgatattgtctgatgatggtgtcgcatgcatagactcagccgttgcaaaggctggagctgttgctgatcttgatggtttccgagacattttgttttaccaaaacaatgtttacatggcagactcaaagtgttggacagaaactgacatgaatgttggccctcatgaattttgctcacagcatactgtgttagcagagcatgatggtaaaccttactacttaccttacccagatgtctctcgcattctgggtgcatgcatctttgtggatgacgttaacaaggctgaccctgttcagaaccttgaacgttacatctcacttgcaattgatgcatatcccctcaccaaggttgaccctattaagggtaaagtcttttatttgttactagactacatacgtgttcttgctcaggagttacaggacggtatccttgatgctttccaatcactcactgacatgtcgtatgtaaataactttatgaatgaggccttttatgctcagatgtatgagcaaagtcctacactacaggccagcggtgtttgtgtggtgtgtaattcacccactatactgcgctgtggtgattgcattcgtcgaccactactttgttgcgtctgtgcctaccagcatgttacgcagactacacataaacgtatcattgctatcaacaactacatctgtagtgttgagaattgcaatgaggacaatgttgaaaaacttttcatttctggcactgcgatttattgtgagaatcacaaacccacgctgtgcatacccattgtagctaatggttctgtttttggtatctatcgccacactgcccgtggtagtgatgacatagacctctttaacgagcttgctacatctaactatgacactattgaaccttatcagaaggccaatcgtgcacctttatcacttatgctcttcgctgctgaaaccattaaggcactcgaggagtctatcaagaagtcatatgctaccgcaaccgtcaaggatgtgtatgaccaacgcttcattaaacttctatgggaacagggtaaaaagccgccacccataacgaagaaccacattttcactggctaccattttaacaagaatggaaaaacccaagttggtgattacattcttgctaaaacagatggcagtgacacttatacttacagaggaacatctacctacaaactccaaacaggtgatgttctagtcttaatggcacatgttgttacaccgctctcagcaccccctgtgttaacgcagacaacatatgtcagaaaatcacttttacccgactctgttggtgcgtcttattatgtgcaacattttaagtcatataatgagatagctatgcagagggttacaacagtattaggtccaccaggcacaggtaagtcaacctttgctattggtttggctaagtactttcccagtgcacgtatttgctacactgcgtcttcgcatgcagcaatcgatgcactctgtgaaaaagctttcaagacaatacctgtaggccaatgcagtcgtatcgtacccacacgtacaactgttgagtgctttcaggagtttgtcgtaaataacacaactgcacagtatatcttctcgactatcaatgccttacctgacattaagtgtgacattgtagttgtagatgaggtttctatgttgaccaattatgagctttcctctgtgaatgctcgtttggtttacaatcacattgtgtatgttggtgatccttatcagttaccttcacctagaactatgcttacgtctggccagctttcgccagctgactataacgtagttactgatataatggtacatgcaggagcggatgttatgctcgacatgtgctacagatgcccacgtgaaatcgttgagacagtgtctaaacttgtctacgataacaaactaaaagcggcgaaaccgaactcaagacagtgttacaagaccattgtgaactttggtcctggagacgttgctcatgagggacaatctgcctacaacgaagcacagttgcgtttcgcactcgcatttagacaacaaaagcggtgggataacgtgactttcatatctccatataatgctatgaatgtgaaagcatccttagcaggtttctctactcagaccgttgactcttctcaaggttctgagtatgattatgttatcttttgcgtgaccactgattcagcacacgcacttaacatggctcgtttgaacgttgcccttacacgcgcaaagataggtatccttgtggtgtttaggcaggcaaacgaactttacaatagtttgcagtttgaatctattgattcacagcttcagtcgagtgctgagaaaaacctcacaccactgtttaagcgctgcggctatgagtataatggcgtccatccagctcatgctttgacctggcatgattgtggtgcagagtaccgctgtgaggagccacttgctaaattagtaggagttgccgatggcactcttatatcatacaaaaccctagtatccacacttgggtttcttccatcacttaaaattgatgcatatcataatatgttcctaacacgtgacgcgtgtcgcacctatgttcagagttggatcggcatagatgttgaagcagcacacgccataaaacctaacaccgggactaacctgccattgcaaataggttttagtaccggaaagaatttttcagtcactccagagggaatttgggtaaacgagcacggatcttgcactgagcccgtccctgccaaaatacctcctggagaacaatttcgtcaccttaaaaaggacatgcgccaggcgcgtccttggaaggttgttcgacgtgagattgctactcacattgctgaggtagctcctcacactgattatatatgctttgtcacttgggctcaccagcttgagctagcgacaatgcgctactttgtcaaactaggtatggaagagaaatgcttttgtggcaggcgggcttgtttcactaatggaactgagttcgcttgcaaagcacaccattctctcaccattccacaatgtgattatgtgtacaatccattcctcatcgacgtggctacgtggggattctcgggacggctttccaccaaccatgacgctgtgtgcacatatcatgctaatgcccatgttgcatcagctgatgcaatcatgacggtatgtttagctatccatgaactgttcagtactgttgactggaactttgaatttccagtaactgctgagcaatcgcaacttaacaaggcctgtcgcttagtacaggcgaattacttaaatatactactcactacaaccaaagccacggtggttcacgatattggtaacccaaaaggtatccctatcgtgcgcaaacctggtgttaaatatcacttctatgatcaagcacccattgtcaaacacgttcaaaaactaaagtacaagccagagatggaggcccgtttcaccgatggtttgactatgttttggaattgtaatgttgacacataccctgctaacgcccttgtgtgccgctacgacactcatcggcagaagcatttaattggacctaatggttcagcactatatgttaataagcatgcttttctcacccctgagatgcatacttatgctacacataaactcaacttggctccactcatctactactccaccacagattgtagtagtgaacagcctattgttgttacctacagagattgtgtcacccggtgcaatactggaaaaactctctgtccaaatcatgctcttgaataccaagagtttatcaatgcatacaatctcatggctcgccatggatttaatgtttacataccacgcaatgtcaacgtttacaactgttggcttactttcactaatctccaaaaccttgaaaacttagcttacaactgttattataagaactgcaatgctcacgttgatgggcagcttgatgtagttattaataataacgctgtatatgctaaggtcgacaataatcttgtcaaacttttcgacaaccgcactaacttacctgtctcagtggcctttgaacattacactaacaggcatacccgttcactgccaactacacagctgttatctggtttaggcgtaaccgccaccagaaatttcactgtgtggttcgacaatgatacaattttccaatacactattaatgtatctacgtatactgacatcgaccctagtacccatgttgtcctctgtgatgataggtacggaacagattggagtcagtttaaccaacttcctaatgcagtattcctcaccaaaactaaggtgaagaaaacagaaccgtttgtttgtacagcactgaccctaaatggcgtcgccattgacggtgaagagctatacatctatgtacgctataacaatcaactgaccacatttgctactacttgtacacagggtagaaatgttgagcagtttatacctaaaacacctatggaaagagacttccttgagatgtctcaacagtccttcatccagcaacatcaattgcaggaactgggtgttgaacacattatctatggtgatgattccagtccagtcattggcggaactcacacacttatctcactagttaaaaacaagtttgaacatcagcttgtcaaccatgtttacaacccagtccagaactgtgttgttacctcacctaacgcaagctccaagaacgtttgcactgttcttgatgttcttcttgatgactacattgacatcataagacaagcacatgccagttacacaagtaaatccaaagtattcactgtgtcaattgacaaccaacaaattagattcatgctttggcatgatgagcaagtcaagacttgctacccaatcttacagtcacttaccaatggttaccagatgccatctgtgtacaaaacattggttactgacttacaaccagctgacatccctaattatcattcctacaccccccgggtgcctggagtagttaagaatgttatcaagtaccgccaacttttcaactacatagttaaaaaggataggttggcagtaccacacaatatgaccgtattacaccttggagctgcatctgcactaggtacagcaccaggttcttcagtcataaaacaaatgtttcctgaaggaactgttcttattgacctcgatataagagagttcacttcagatgctaaccaaataatagttacagactacagaacttacataccaccacaccacgtagacgtcatattttctgacctctactgttgtgatgacatacacttctttgacaatctaataaggatagttaaggagaggctcgccctcggtggttctatctttgttaagataactgaacattcattctcacccgaactctactcacttgcgggttggttcgatgattatcaactattttgcacagcagtcaatgcctcgtcttcagaagcatttttatgctgttttaattatttggggcttgctaaggaaaacattaatggttttaacttacatgcttcctatattcaatggcgcaatgaaatagcgttgacaccaacctattctcctttagcggacaacccggctacggcctgtaagctaaaagcaacgcctattatctcggctcgtgagttagagaagaagcctattcttcgctatctcgttgcatcagggcgccttcttgtgaggccaccagaatgcagagagctctattgattatgaccttattttgtctcgttcgagcaaagtttgctgatgatctactcgatttgctcaccttcccgggtgcacatcgcttcttacataaacccacgaggaattccagcagtctctactcgcgggctaataataattttgatgttggcgttcttcctggctaccccactaagaacgttaacctcttctcaccacttactaactccactttgcccattaatggccttcatcggagttaccaaccactcatgctgaattgtcttactaaaataactaaccacactctcagcatgtatctcctacctagtgagatacaaacttatagctgcggcggtgccatggttaaataccagacacatgatgcagttcgtatcattttagacctcactgccactgaccacatctctgttgaagtcgttggccaacatggtgaaaattatgtgtttgtttgcagtgagcagtctacctacaccactgcattacacaaatctaccttcttctcacttaattctgagctttattgctttactaataacacctacttaggtattcttccacctgatttaactgactttacggtctaccgtactggtcagttctatgctaatggttaccttttaggtactttacctattacggttaactatgttaggttgtatcggggtcatttgtctgccaatagtgcccactttgcccttgcaaacctaaccgatacactcataacacttaccaatactactatatcgcaaatcacttattgtgataagtctgtagttgattcaatagcatgccagcgctcttctcacgaagtggaggatgggttttactccaaccctaaatctgccgttagagctaggcaacgtactattgttacactacctaagctccctgagcttgaagtagtgcagttaaatatttctgcacacatggattttggcgaagccagacttgacagcgttaccatcaatggtaacacatcctattgtgtcactaagccttacttcaggcttgaaactaactttatgtgtacaggttgcactatgaatctgcgcactgatacctgtagttttgacctgtcagcagtaaacaatggcatgtcattctctcaattctgtctaagcactgaatctggtgcttgtgagatgaaaattattgttacctacgtatggaattacttgctaaggcagcgtttgtatgttactgctgtagagggccagactcacactggaaccacttcagtacatgcaacagacacttctagtgtaatcactgatgtctgcactgactacactatctatggagtctctggtactggcattattaagccatcagatctcttattgcacaatggcatagcattcacctctccaacaggtgagctttatgcatttaaaaatataaccactggcaaaacccttcaggtcttaccgtgtgaaaccccttctcaactgattgtgataaacaacaccgttgtcggtgctatcacatccagtaattcaactgaaaataataggtttactactactattgtcacacctactttcttttattccacaaatgccaccactttcaactgcactaagcctgttttgtcctatggacctatcagcgtgtgtagtgatggtgcaattgtgggaacatccacattacagaatactcgaccatccatagtttcactatacgatggcgaagttgaaataccatctgcattttctctttccgttcagacggagtacttgcaagttcaagcagagcaagttatagttgattgtcctcagtatgtatgcaatggcaacagccgttgtctacaattactggcacaatacacctcagcttgctctaagattgaagcagctctgcattcctctgcacagttggatagcagagagattataaatatgtttcaaacatcaacacagtccttgcagttagctaatattaccaacttcaagggtgactacaattttagcagcatactaaccaccagaattggtggcagatctgctattgaagaccttctttttaataaagttgttactagtggccttggcactgttgatcaggactacaaatcctgctctagagacatggccatcgctgacttagtttgttcccagtattacaatggcatcatggttctacctggtgttgttgatgctgagaaaatggcaatgtatactggctctcttactggagctatggtatttggaggactgactgccgcagcggcaataccatttgccacggcagtacaagctcgcctcaattatgtcgcactgcaaacaaatgtactacaagaaaaccagaaaattcttgcagaatcatttaaccaagcagttggcaatatatcacttgcactatcttctgttaatgatgccatccagcaaacttctgaggctcttaacaccgtagctattgctattaaaaagattcaaacagttgttaaccagcagggcgaggcattatcacacctgactgcacagctgtcaaacaattttcaagcaatttcgacttctattcaagacatttacaaccgtcttgaggaagtagaggctaaccagcaagttgaccgtctcatcacaggacggttggctgcacttaatgcatatgttactcagttactcaatcagatgtctcagattagacaatctcgattgttagctcagcaaaagattaatgagtgtgtcaaatcacagtcgtccagatacggtttctgtggaaatggcacacacatcttctcacttacacagactgcaccaaatggcatatttttcatgcatgcagtgctagtacccaacaaattcacacgtgtcaacgcttctgccggcatttgtgtggataatacgagaggctactcattgcagcctcaacttatactctaccagtttaataactcctggagagttacacctagaaatatgtatgaacccagactgccccggcaggctgatttcatacaattaactgattgcagcgttactttttacaacaccaccgctgctaatcttcccaatattatccctgacattatagatgtcaatcaaacagtcagtgatattattgacaatttacctacagcaacacctcctcagtgggatgttggtatctataacaacactattctcaacctcaccgttgagattaatgatctacaagagcggtctaaaaacctctcacagattgcagatcgtttacaaaattatattgacaatcttaacaatactctagttgaccttgaatggctcaacagagtggaaacttaccttaaatggccgtggtatatatggcttgccattgccctggctcttattgcatttgtgacaatcctcataacaatctttctttgtactggttgttgtggtggttgctttggttgttgtggcggttgttttggccttttctctaagaagaaaaggtataccgacgaccaaccaacaccgtcctttaagtttaaggaatggtagtcgacgactgggccgttaccatccctggacaatatattattgctatactagttgtcatctgcattggtgtggcactactttttattaatacttgcttagcttgtgttaaattattttacaagtgctacctaggggcagcatatcttgttaggcctattatagtgtactactccaagccgaaccccgtacctgaggatgagtttgtaaaagtacaccaatttcctagaaacactcactatgtctgacgcagaagagtggcaaattattgttttcattgcgatcatatgggcgcttggcgtcatcctccagggaggctatgccacgcgtaatcgtgtgatctatgttattaaacttattctgctttggctgctccaacccttcaccctagtggtgaccatttggaccgcagtcgacagatcatctaagaaggacgcagttttcattgtgtccataatttttgccgtactgaccttcatatcctgggccaagtactggtatgactcaattcgtttattaatgaaaaccagatctgcatgggcactctcacctgagagtagactccttgcagggattatggatccaatgggtacatggaggtgcattcccattgaccacatggctccaattctcacaccagtcgttaagcatggcaagctcaagctacatgggcaagagctggccaatggcatatcagtcagaaatccgccacaggatatggtgatagtgtcaccaagtgacacctttcactacacttttaagaaacctgtggaatcaaacaacgatccagaatttgctgttctgatataccagggtgaccgcgcttcaaacgctggacttcacaccataaccacttcaaaggccggtgacgctcgcctgtataagtatatgtaatgtgcaactgccatctgcagctgcgagatttatatagattgtgcaataagcggcacatcagaagagaggatgttcctgagcttattgaccctctcgttaaaactcgctgttttgcttacagtctcgtggttcttgctaatgctaatccaattgcatttagcatactacctcggaaaattcttatcaatggtgagcctttactgcttgaatatggtagcatatatggtaaagactttatcattcgaccatcgctccaagtcattcttgaagatgaattaaattaaagttttgacaccaatctatcatggctgcaccagtagtccctactactgacgcgtcttggtttcaggtgctcaaagctcaaaacaaaaaggctactcatcctcagtttcgtggcaatggagttccgcttaactccgccatcaaacccgttgaaaaccatggctactggctgcgttacaccagacaaaagccaggtggtactcccattcctccatcctatgccttttattatactggcacaggtcccagaggaaatcttaagtatggtgaactccctcctaatgataccccagcaaccactcgtgttacttgggttaagggttcgggagctgacacttctattaaacctcatgttgccaaacgcaaccccaacaatcctaaacatcagctgctacctctccgattcccaaccggagatggcccagctcaaggtttcagagttgaccccttcaacgctagaggaagacctcaggagcgtggaagtggcccaagatctcaatctgttaactccagaggcacaggcaatcagcccaggaaacgcgaccaatctgcaccagctgcggtacgtcgtaagacccagcatcaagctcccaagcggactttacccaagggtaaaaccatttctcaggtatttggcaaccggtctcgtactggtgccaatgtcggctctgcagacactgagaagacgggtatggctgatcctcgcatcatggctctagccagacatgtgcctggtgttcaggaaatgctttttgctggccaccttgagagcaactttcaggcgggggcaattacccttaccttctcctactcaatcacagtcaaggagggttctcctgactatgagagacttaaggatgcgctcaatacggtcgttaaccagacctatgagccacctaccaaaccaactaaggacaagaagcctgacaaacaagaccagtctgctaaacccaaacagcagaagaaacctaaaaaggtaactctgccagcagacaaacaggattgggagtgggatgatgcttttgagataaagcaggaatcagcagcgtagacatcaatctatgtctgttaaacccacccaactccactcaaatatctctttggttccagagagtcgtagtgtatagccagagagccagtcagagggcgctatcatgcaaactagggctggctactctagcacagaatcacatcccgataatcaacagtgctagaaggttgattataccatttaatatgccgaggccacgcggagtacgatcgagggtacagcataatctcaacttttgttgagccacaattttaatcctaattggagaaggccaaaggactgtactacttttgtgggtgtagcagtcgcccagtgggaaagcgccaactaggttacaattgtggtggggacaaattaggggaaattaaattggcttataggggggatggagcag